SEQUENCE LISTING (19 sequences)

SEQ ID NO: 1 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 2 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 219 aa, synthetic construct
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 4 - protein, 51 aa, synthetic construct
SDPKPENPACPWTVLPAGDLPTHDGYLPSNIDDLPSHEAPLADSLEELEPQ

SEQ ID NO: 5 - protein, 191 aa, synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 6 - protein, 285 aa, synthetic construct
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 7 - protein, 221 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNSDPKPENPACPWTVLPAGDLPTHDGYLPSNIDDLPSHEAPLADSLEELEPQ

SEQ ID NO: 8 - protein, 26 aa, synthetic construct
VTGISLLPPLGVAISVIIIFYCYRVN

SEQ ID NO: 9 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 10 - protein, 219 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 11 - protein, 263 aa, synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 308 aa, synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 340 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHYQVSFCLVMVLLFAVDTGLYFSVKTNIRSSTRDWKDHKFKWRKDPQDK

SEQ ID NO: 14 - protein, 336 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLFFPPGYQVSFCLVMVLLFAVDTGLYFSVKTNIRSSTRDWKDHKFKWRKDPQDK

SEQ ID NO: 15 - protein, 335 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 16 - protein, 147 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 17 - protein, 126 aa, synthetic construct [sequence omitted]

SEQ ID NO: 18 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 19 - protein, 207 aa, synthetic construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

(19) United States
(12) Patent Application Publication
(10) Pub. No.: US 2025/0032547 A1
(43) Pub. Date: Jan. 30, 2025

(54) EFFECTOR CELLS AND USE THEREOF FOR ALLOGENEIC ADOPTIVE CELL THERAPIES IN SOLID TUMORS

(71) Applicant: FATE THERAPEUTICS, INC., San Diego, CA (US)

(72) Inventors: Emily CARRON, San Diego, CA (US); Eigen PERALTA, San Diego, CA (US); Bahram VALAMEHR, San Diego, CA (US); Hui-Yi CHU, San Diego, CA (US); Tom Tong LEE, San Diego, CA (US)

(21) Appl. No.: 18/917,836

(22) Filed: Oct. 16, 2024

Related U.S. Application Data
(63) Continuation of application No. 18/702,764, filed on Apr. 18, 2024.
(60) Provisional application No. 63/257,984, filed on Oct. 20, 2021.
(60) Provisional application No. 63/328,952, filed on Apr. 8, 2022.

(51) Int. Cl. A61K 35/17; A61K 39/00; A61P 35/00; C07K 14/715; C07K 14/725; C07K 14/735; C12N 5/074; C12N 5/0783

(52) U.S. Cl. CPC A61K 35/17; A61K 39/4611; A61K 39/4613; A61K 39/4631; A61P 35/00; C07K 14/7051; C07K 14/70535; C07K 14/7155; C12N 5/0636; C12N 5/0646; C12N 5/0696; C12N 2501/15; C12N 2501/2302; C12N 2502/30; C12N 2506/45; C12N 2510/00

(57) ABSTRACT

Provided are methods and compositions for obtaining functionally enhanced derivative effector cells obtained from directed differentiation of genomically engineered iPSCs. In various embodiments, the derivative cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATIONS

This application claims priority to U.S. Provisional Application Ser. No. 63/257,984, filed Oct. 20, 2021, and to U.S. Provisional Application Ser. No. 63/328,952, filed Apr. 8, 2022, the disclosure of each of which is hereby incorporated by reference in their entireties.

### INCORPORATION BY REFERENCE OF SEQUENCE LISTING

The Sequence Listing titled 184143-637601_SL.xml, which was created on Oct. 19, 2022 and is 21,239 bytes in size, is hereby incorporated by reference in its entirety.

### FIELD OF THE INVENTION

The present disclosure is broadly concerned with the field of off-the-shelf immunocellular products. More particularly, the present disclosure is concerned with the strategies for developing multifunctional effector cells capable of delivering therapeutically relevant properties in vivo. The cell products developed under the present disclosure address critical limitations of patient-sourced cell therapies.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit therefrom. There is also a need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, there remain significant opportunities to harness the full potential of T and NK cells, or other lymphocytes in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of embodiments of the present invention to provide methods and compositions to generate derivative non-pluripotent cells differentiated from a single cell derived iPSC (induced pluripotent stem cell) clonal line, which iPSC line comprises one or several genetic modifications in its genome. Said one or several genetic modifications include, in some embodiments, DNA insertion, deletion, and substitution, and which modifications are retained and remain functional in subsequently derived cells after differentiation, expansion, passaging and/or transplantation.

The iPSC-derived non-pluripotent cells of the present application include, but are not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells. The iPSC-derived non-pluripotent cells of the present application comprise one or several genetic modifications in their genome through differentiation from an iPSC comprising the same genetic modifications. In some embodiments, the engineered clonal iPSC differentiation strategy for obtaining genetically engineered derivative cells benefits from the developmental potential of the iPSC in a directed differentiation that is not significantly adversely impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Some aspects of the present invention provide genome-engineered iPSCs obtained using a method comprising (I), (II) or (III), reflecting a strategy of genomic engineering subsequently to, simultaneously with, and prior to the reprogramming process, respectively:
(I): genetically engineering iPSCs by one or both of (i) and (ii), in any order: (i) introducing into iPSCs one or more construct(s) to allow targeted integration at selected site(s); (ii) (a) introducing into iPSCs one or more double-stranded break(s) at selected site(s) using one or more endonuclease capable of selected site recognition; and (b) culturing the iPSCs of step (I)(ii)(a) to allow endogenous DNA repair to generate targeted in/dels at the selected site(s); thereby obtaining genome-engineered iPSCs capable of differentiation into partially or fully differentiated cells.
(II): genetically engineering and reprogramming non-pluripotent cells to obtain the genome-engineered iPSCs by: (i) contacting non-pluripotent cells with one or more reprogramming factors, and optionally a small molecule composition comprising a TGF$ receptor/ALK inhibitor, a MEK inhibitor, a GSK3 inhibitor and/or a ROCK inhibitor to initiate reprogramming of the non-pluripotent cells; and (ii) introducing into the non-pluripotent cells during the reprogramming of step (IIxi) one or both of (a) and (b), in any order: (a) one or more construct(s) to allow targeted integration at selected site(s); (b) one or more double-stranded break(s) at selected site(s) using at least one endonuclease capable of selected site recognition, then the cells of step (II)(ii)(b) are cultured to allow endogenous DNA repair to generate targeted in/dels at the selected site(s); as such the obtained genome-engineered iPSCs comprise at least one functional targeted genomic edit and said genome-engineered iPSCs are capable of differentiation into partially or fully differentiated cells.
(III): genetically engineering non-pluripotent cells for reprogramming to obtain genome-engineered iPSCs by (i) and (ii): (i) introducing into non-pluripotent cells one or both of (a) and (b), in any order: (a) one or more construct(s) to allow targeted integration at selected site(s); (b) one or more double-stranded break(s) at selected site(s) using at least one endonuclease capable of selected site recognition, wherein the cells of step (III)(i)(b) are cultured to allow endogenous DNA repair to generate targeted in/dels at the selected sites; and (ii) contacting the cells of step (III)(i) with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted editing at the selected site(s); thereby obtaining genome-engineered iPSCs comprising at least one functional targeted genomic edit, and said genome-engineered iPSCs are capable of being differentiated into partially differentiated cells or fully-differentiated cells.

In one embodiment of the above method, the at least one targeted genomic edit(s) at one or more selected site(s) comprises insertion of one or more exogenous polynucleotides encoding safety switch proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof. In some embodiments, the exogenous polynucleotides for insertion are operatively linked to (1) one or more exogenous promoters comprising CMV, EF1α, PGK, CAG, UBC, or other constitutive, inducible, temporal-, tissue-, or cell type-specific promoters; or (2) one or more endogenous promoters comprised in the selected site(s) comprising AAVS1, CCR5, ROSA26, collagen, HTRP, H11, GAPDH, TCR or RUNX1, or other locus meeting the criteria of a genome safe harbor. In some embodiments, the genome-engineered iPSCs generated using the above method comprise one or more different exogenous polynucleotides encoding protein comprising caspase, thymidine kinase, cytosine deaminase, modified EGFR, or B-cell CD20, wherein when the genome-engineered iPSCs comprise two or more suicide genes, the suicide genes are integrated in different safe harbor loci comprising AAVS1, CCR5, ROSA26, collagen, HTRP, H11, GAPDH, TCR or RUNX1. In some embodiments, the exogenous polynucleotide encodes a partial or full peptide of IL2, IL4, IL6, IL7, IL9, IL10, IL11, IL12, IL15, IL18, IL21, and/or respective receptors thereof. In some embodiments, the partial or full peptide of IL2, IL4, IL6, IL7, IL9, IL10, IL11, IL12, IL15, IL18, IL21, and/or respective receptors thereof encoded by the exogenous polynucleotide is in the form of a fusion protein.

In some other embodiments, the genome-engineered iPSCs generated using the method provided herein comprise an in/del at one or more endogenous genes associated with targeting modalities, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof. In some embodiments, the endogenous gene for disruption, either reduction or knockout, comprises B2M, TAP1, TAP2, tapasin, NLRC5, CIITA, RFXANK, CIITA, RFX5, RFXAP, TCRα or TCRβ constant region. NKG2A, NKG2D, CD38, CD25, CD69, CD71, CD44, CD58, CD54, CD56, CIS, CBL-B, SOCS2, PD1, CTLA4, LAG3, TIM3, or TIGIT. In some embodiments, the endogenous gene for disruption comprises at least one of NLRC5, PD1, LAG3, and TIM3.

In yet some other embodiments, the genome-engineered iPSCs generated using the method provided herein comprise a caspase encoding exogenous polynucleotide at AAVS1 locus, and a thymidine kinase encoding exogenous polynucleotide at H11 locus.

In still some other embodiments, approach (1), (11) and/or (III) further comprises: contacting the genome-engineered iPSCs with a small molecule composition comprising a MEK inhibitor, a GSK3 inhibitor and a ROCK inhibitor, to maintain the pluripotency of the genome-engineered iPSCs. In one embodiment, the obtained genome-engineered iPSCs comprising at least one targeted genomic edit(s) are functional, are differentiation potent, and are capable of differentiating into non-pluripotent cells comprising the same functional genomic edit(s).

Accordingly, in one aspect, the present invention also provides a cell or population thereof, wherein: (i) the cell is (a) an immune cell; (b) an induced pluripotent cell (iPSC), a clonal iPSC, or an iPS cell line cell; or (c) a derivative cell obtained from differentiating the iPSC; (ii) the cell comprises an exogenous polynucleotide encoding a signaling redirector receptor (SRR) that comprises a partial or full peptide of an extracellular domain (ECD) of a signaling receptor and a partial or full peptide of an intracellular domain (ICD) of a cytokine receptor, wherein the signaling receptor and the cytokine receptor are different molecules, (iii) the cell has improved resistance to cytokine immunosuppression in an adoptive cell therapy for solid tumors; and (iv) the cell optionally further comprises one or more of: (a) an exogenous polynucleotide encoding a CAR (chimeric antigen receptor); (b) an exogenous polynucleotide encoding a CD16 or a variant thereof, (c) CD38 knockout; and (d) an exogenous polynucleotide encoding cytokine signaling complex comprising a partial or full peptide of a cell surface expressed exogenous cytokine and/or a receptor thereof. In various embodiments, the signaling redirector receptor comprises: (a) a partial or full peptide of the extracellular domain (ECD) of a signaling receptor comprising transforming growth factor beta receptor (TGFβR), programmed cell death 1 (PD1), CTLA4, IL10R, IL4R, or any combination thereof; and (b) a partial or full peptide of the intracellular domain (ICD) of a cytokine receptor comprising IL2Rβ, IL12Rβ, IL18Rβ, IL21Rβ, or any combination thereof. In various embodiments, the signaling receptor comprises TGFβR2, wherein the signaling redirector receptor is a TGFβ-SRR, and (a) the cytokine receptor is IL2Rβ, thereby forming a TGFβR2-IL2Rβ signaling redirector receptor; or (b) the cytokine receptor is IL12Rβ, thereby forming a TGFβR2-IL12Rβ signaling redirector receptor; or (c) the cytokine receptor is IL18Rβ, thereby forming a TGFβR2-IL18Rβ signaling redirector receptor; or (d) the cytokine receptor is IL21R, thereby forming a TGFβR2-IL21R signaling redirector receptor.

In various embodiments of the cell or population thereof, (a) the intracellular domain (ICD) of IL2Rβ comprises an amino acid sequence represented by SEQ ID NO: 2 (NCRNTGPWLKKVLKCNTPDPSKFFSQLSSEHGGDVQKWLSSPFPSSSFSPGGLAPEISPL EVLERDKVTQLLLQQDKVPEPASLSSNHSLTSCFTNQGYFFFHLPDALEIEACQVYFTYD PYSEEDPDEGVAGAPTGSSPQPLQPLSGEDDAYCTFPSRDDLLLFSPSLLGGPSPPSTAPGG SGAGEERMPPSLQERVPRDWDPQPLGPPTPGVPDLVDFQPPPELV LREAGEEVPDAGPRE GVSFPWSRPPGQGEFRALNARLPLNTDAYLSLQELQGQDPTHLV); or (b) the intracellular domain (ICD) of IL12Rβ comprises an amino acid sequence represented by (i) SEQ ID NO: 3 (HYFQQKVFVLLAALRPQWCSREIPDPANSTCAKKYPIAEEKTQLPLDRLLIDWPTPEDPE PLVISEV LHQVTPVFRHPPCSNWPQREKGIQGHQASEKDMMHSASSPPPPRALQAESRQL VDLYKVLESRGSDPKPENPACPWTVLPAGDLPTHDGYLPSNIDDLPSHEAPLADSLEELE PQHISLSVFPSSSLHPLTFSCGDKLTLDQLKMRCDSLML), or (ii) SEQ ID NO: 4 (SDPKPENPACPWTVLPAGDLPTHDGYLPSNIDDLPSHEAPLADSLEELEPQ); or (c) the intracellular domain (ICD) of IL18Rβ comprises an amino acid sequence represented by SEQ ID NO: 5 (YRVDLVLFYRHLTRRDETLTDGKTYDAFVSYLKECRPENGEEHTFAVEILPRV LEKHFGY KLCIFERDVVPGGAVVDEIHSLIEKSRRLIIVLSKSYMSNEVRYELESGLHEALVERKIKIIL IEFTPVTDFTFLPQSLKLLKSHRVLKWKADKSLSYNSRFWKNLLYLMPAKTVKPGRDEP EVLPVLSES); or (d) the intracellular domain (ICD) of IL21R0 comprises an amino acid sequence represented by SEQ ID NO: 6 (SLKTHPLWRLWKKIWAVPSPERFFMPLYKGCSGDFKKWVGAPFTGSSLELGPWSPEVPS TLEVYSCHPPRSPAKRLQLTELQEPAELVESDGVPKPSFWPTAQNSGGSAYSEERDRPYG LVSIDTVTVLDAEGPCTWPCSCEDDGYPALDLDAGLEPSPGLEDPLLDAGTTVLSCGCVS AGSPGLGGPLGSLLDRLKPPLADGEDWAGGLPWGGRSPGGVSESEAGSPLAGLDMDTF DSGFVGSDCSSPVECDFTSPGDEGPPRSYLRQWVVIPPPLSSPGPQAS); or (e) the extracellular domain (ECD) ofTGFβR comprises an amino acid sequence represented by SEQ ID NO: 1 (TIPPHVQKSVNNDMIVTDNNGAVKFPQLCKFCDVRFSTCDNQKSCMSNCSITSICEKPQ EVCVAVWRKNDENITLETVCHDPKLPYHDFILEDAASPKCIMKEKKKPGETFFMCSCSSD ECNDNIIFSEEYNTSNPDLLLVIFQ). In some embodiments of the cell or population thereof, the TGFβR2-L12Rβ signaling redirector receptor comprises an amino acid sequence having sequence identity of at least 80%, 85%, 90%, 95%, or 97%, 98%, or 99% to a sequence represented by SEQ ID NO: 7 (TIPPHVQKSVNNDMIVTDNNGAVKFPQLCKFCDVRFSTCDNQKSCMSNCSITSICEKPQ EVCVAVWRKNDENITLETVCHDPKLPYHDFILEDAASPKCIMKEKKKPGETFFMCSCSSD ECNDNIIFSEEYNTSNPDLLLVIFQVTGISLLPPLGVAISVIIIFYCYRVNSDPKPENPACPWT VLPAGDLPTHDGYLPSNIDDLPSHEAPLADSLEELEPQ; termed specifically as TGFβR2-trIL12Rβ throughout the application), wherein an amino acid sequence represented by SEQ ID NO: 8 (VTGISLLPPLGVAISVIIIFYCYRVN) comprised in SEQ ID NO: 7 is variable.

In various embodiments, the cell further comprises: (i) at least one of the genotypes listed in Table 1; (ii) HLA-1 deficiency and/or HLA-11 deficiency. (iii) introduction of HLA-G or non-cleavable HLA-G, or knockout of one or both of CD58 and CD54; (iv) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (v) introduction of at least one of HLA-E, 4-1BBL, CD3, CD4, CD8, CD16, CD47, CD64, CD113, CD131, CD137, CD80, PDL1, A2AR, TCR, chimeric fusion receptor (CFR), Fc receptor, an antibody or functional variant or fragment thereof, a checkpoint inhibitor, an engager, and surface triggering receptor for coupling with bi- or multi-specific or universal engagers. In various embodiments, the cell comprises HLA-I deficiency and/or HLA-II deficiency, and optionally, wherein the cell comprises an exogenous polynucleotide encoding HLA-G, HLA-E, or a variant thereof; and/or comprises deletion or disruption of one or both of CD54 and CD58. In certain embodiments, the HLA-I deficiency comprises deletion or disruption of at least one of: B2M, TAP1, TAP2, and Tapasin; and/or wherein the HLA-II deficiency comprises deleted or reduced expression of at least one of: CIITA, RFX5, RFXAP, and RFXANK.

In various embodiments of the cell or population thereof, the derivative cell: (a) comprises a derivative CD34⁺ cell, a derivative hematopoietic stem and progenitor cell, a derivative hematopoietic multipotent progenitor cell, a derivative T cell progenitor, a derivative NK cell progenitor, a derivative T cell, a derivative NKT cell, a derivative NK cell, a derivative B cell, or a derivative effector cell having one or more functional features that are not present in a counterpart primary T, NK, NKT, and/or B cell; (b) is used as an allogeneic effector cell, wherein the effector cell is a derivative NK cell or a derivative T cell having at least one of the following characteristics comprising: (i) improved persistency and/or survival; (ii) increased resistance to activated recipient immune cells; (iii) increased cytotoxicity; (iv) improved tumor penetration; (v) enhanced or acquired ADCC; (vi) enhanced ability in migrating, and/or activating or recruiting bystander immune cells, to tumor sites; (vii) enhanced ability to reduce tumor immunosuppression; (viii) improved ability in rescuing tumor antigen escape; and (ix) reduced fratricide, in comparison to its native counterpart cell obtained from peripheral blood, umbilical cord blood, or any other donor tissues.

In various embodiments of the cell or population thereof, the exogenous CD16 comprises at least one of: (a) a high affinity non-cleavable CD16 (hnCD16) or a variant thereof; (b) F176V and S197P in ectodomain domain of CD16; (c) a full or partial ectodomain originated from CD64: (d) a non-native (or non-CD16) transmembrane domain: (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide. In certain embodiments, (a) the non-native transmembrane domain is derived from a CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide; (b) the non-native stimulatory domain is derived from a CD27, CD28, 4-1BB, OX40, ICOS, PD-1, LAG-3, 2B4, BTLA, DAP10, DAP12, CTLA-4, or NKG2D polypeptide; (c) the non-native signaling domain is derived from a CD3ζ, 2B4, DAP10, DAP12, DNAM1, CD137 (4-1BB), IL21, IL7, IL12, IL15, NKp30, NKp44, NKp46, NKG2C, or NKG2D polypeptide; or (d) the non-native transmembrane domain is derived from NKG2D, the non-native stimulatory domain is derived from 2B4, and the non-native signaling domain is derived from CD3ζ.

In various embodiments of the cell or population thereof, the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with a cytokine signaling complex comprising a partial or full peptide of a cell surface expressed exogenous cytokine and/or a receptor thereof, optionally in separate constructs or in a bi-cistronic construct; (ix) co-expressed with a checkpoint inhibitor, optionally in separate constructs or in a bi-cistronic construct; and/or (x) optionally inserted at: a TRAC or a TRBC locus, and/or is driven by an endogenous promoter of TCR, and/or the TCR is knocked out by the CAR insertion; a safe harbor locus; or a gene locus intended for disruption. In various embodiments of the cell or population thereof, the CAR is: (i) specific to at least one of CD19, BCMA, B7H3, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1; and/or (ii) specific to any one ofADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD I33, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), Mucin 1 (Muc-1), Mucin 16 (Muc-16), Mesothelin (MSLN), NKCSI, NKG2D ligands, c-Met, cancer-testis antigen NYESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen.

In various embodiments of the cell or population thereof, the cytokine signaling complex comprises: (a) a partial or full peptide of at least one of IL2, IL4, IL6, IL7, IL9, IL10, IL11, IL12, IL15, IL18, IL21, and/or respective receptor(s) thereof; or (b) at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated (IL15Δ); (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ; wherein any one of (i)-(vii) is optionally co-expressed with a CAR in separate constructs or in a bi-cistronic construct; or (c) at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic construct; and optionally, (d) is transiently expressed.

In various embodiments of the cell or population thereof, the cell is a derivative NK or a derivative T cell, wherein the derivative NK cell is capable of recruiting and/or migrating T cells to tumor sites, and wherein the derivative NK cell or the derivative T cell is capable of reducing tumor immunosuppression in the presence of one or more checkpoint inhibitors. In some embodiments, the one or more checkpoint inhibitors are antagonists to one or more checkpoint molecules comprising PD-1, PDL-1, TIM-3, TIGIT, LAG-3, CTLA-4, 2B4, 4-1BB, 4-1BBL, AAR, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR. In particular embodiments, the one or more checkpoint inhibitors comprise: (a) one or more of atezolizumab, avelumab, durvalumab, ipilimumab, IPH4102, IPH43, IPH33, lirimumab, monalizumab, nivolumab, pembrolizumab, and their derivatives or functional equivalents; or (b) at least one of atezolizumab, nivolumab, and pembrolizumab.

In various embodiments of the cell or population thereof, the cell comprises: (i) one or more exogenous polynucleotides integrated in one safe harbor locus or locus intended for disruption; or (ii) more than two exogenous polynucleotides integrated in different safe harbor loci or loci intended for disruption. In some embodiments, the safe harbor locus or loci comprises at least one of AAVS1, CCR5, ROSA26, collagen, HTRP, H11, GAPDH, TCR or RUNX1; or wherein the gene locus or loci intended for disruption comprises at least one of B2M, TAP1, TAP2, tapasin, NLRC5, CIITA, RFXANK, RFX5, RFXAP, TCRa or TCRP constant region, NKG2A, NKG2D, CD38, CD25, CD69, CD71, CD44, CD58, CD54, CD56, CIS, CBL-B, SOCS2, PD1, CTLA4, LAG3, TIM3, orTIGIT.

In another aspect, the invention provides a composition comprising the cell or population thereof described herein. In various embodiments, the composition further comprises one or more therapeutic agents. In particular embodiments, the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In some embodiments, (i) the checkpoint inhibitor comprises: (a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, Rara (retinoic acid receptor alpha). TLR3, VISTA, NKG2A/HLA-E, or inhibitory KIR; (b) one or more of atezolizumab, avelumab, durvalumab, ipilimumab, IPH4102, IPH43, IPH33, lirimumab, monalizumab, nivolumab, pembrolizumab, and their derivatives or functional equivalents, (c) at least one of atezolizumab, nivolumab, and pembrolizumab; or (ii) the therapeutic agents comprise one or more of venetoclax, azacitidine, and pomalidomide. In some embodiments, the antibody comprises: (a) an anti-CD20 antibody, an anti-HER2 antibody, an anti-CD52 antibody, an anti-EGFR antibody, an anti-CD123 antibody, an anti-GD2 antibody, or an anti-PDL1 antibody; or (b) one or more of rituximab, veltuzumab, ofatumumab, ublituximab, ocaratuzumab, obinutuzumab, trastuzumab, pertuzumab, alemtuzumab, cetuximab, dinutuximab, avelumab, daclizumab, basiliximab, M-A251, 2A3, BC69, 24204, 22722, 24212, MAB23591, FN50, 298614, AF2359, CYIG4, DF1513, bivatuzumab, RG7356, G44-26, 7G3, CSL362, elotuzumab, and their humanized or Fc modified variants or fragments and their functional equivalents and biosimilars thereof.

In various embodiments of the composition, the engager comprises: (i) a bispecific T cell engager (BiTE); (ii) a bispecific killer cell engager (BiKE); or (iii) a tri-specific killer cell engager (TriKE); or wherein the engager comprises: (a) a first binding domain recognizing an extracellular portion of CD3, CD5. CD16. CD28, CD32, CD33, CD64, CD89, NKG2C, NKG2D, or any functional variants thereof of the cell or a by-stander immune effector cell; and (b) a second binding domain specific to an antigen comprising any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2. In another aspect, the invention provides for therapeutic use of the composition described herein by introducing the composition to a subject in need of an adoptive cell therapy, wherein the subject has an autoimmune disorder: a hematological malignancy, a solid tumor, cancer, or a virus infection.

In yet another aspect, the invention provides a master cell bank (MCB) comprising the clonal iPSC described herein.

In yet another aspect, the invention provides a method of manufacturing the derivative cell described herein, wherein the derivative cell is an effector cell, and the method comprises differentiating a genetically engineered iPSC, wherein the iPSC comprises an exogenous polynucleotide encoding the signaling redirector receptor described herein: thereby providing the effector cell with improved resistance to cytokine immunosuppression in an adoptive cell therapy for solid tumors. In various embodiments of the method, the signaling redirector receptor comprises: (a) a partial or full peptide of the extracellular domain (ECD) of a signaling receptor comprising transforming growth factor beta receptor (TGFβR), programmed cell death 1 (PD1), CTLA4, IL10R, IL4R, or any combination thereof; and (b) a partial or full peptide of the intracellular domain (ICD) of a cytokine receptor comprising IL2Rβ, IL12Rβ, IL18Rβ, IL21Rβ, or any combination thereof; wherein the genetically engineered iPSC is a single cell, a clonal cell, or a cell line cell. In various embodiments of the method, the signaling receptor comprises TGFβR, and (a) the cytokine receptor is IL2Rβ, thereby forming a TGFβR2-IL2R43 signaling redirector receptor; or (b) the cytokine receptor is IL12R0, thereby forming a TGFβR2-IL12Rβ signaling redirector receptor; or (c) the cytokine receptor is IL18Rβ, thereby forming a TGFβR2-IL18Rβ signaling redirector receptor; or (d) the cytokine receptor is IL21R, thereby forming a TGFβR2-IL21R signaling redirector receptor. In particular embodiments of the method, the cell further comprises: (i) at least one of the genotypes listed in Table 1; (ii) HLA-I deficiency and/or HLA-II deficiency; (iii) introduction of HLA-G or non-cleavable HLA-G, or knockout of one or both of CD58 and CD54; (iv) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; (v) introduction of at least one of HLA-E, 4-1BBL, CD3, CD4, CD8, CD16, CD47, CD64, CD113, CD131, CD137, CD80, PDL1, A_{2A}R, TCR, chimeric fusion receptor (CFR), Fc receptor, an antibody or functional variant or fragment thereof, a checkpoint inhibitor, an engager, and surface triggering receptor for coupling with bi- or multi-specific or universal engagers. In some embodiments of the method, the cell comprises HLA-1 deficiency, and/or HLA-II deficiency; and optionally wherein the cell comprises an exogenous polynucleotide encoding HLA-G, HLA-E, or a variant thereof; or comprises deletion or disruption of one or both of CD54 and CD58.

In various embodiments of the method of manufacturing, the method further comprises genomically engineering an iPSC to knock in: (a) a polynucleotide encoding the signaling redirector receptor; and optionally, (b) the exogenous polynucleotide encoding the chimeric antigen receptor (CAR); and optionally (c) the exogenous polynucleotide encoding the CD16 or a variant thereof; and optionally further comprising genomically engineering the iPSC: (i) to knock out CD38, (ii) to knock out one or both of B2M and CIITA, (iii) to knock out one or both of CD58 and CD54, and/or (iv) to introduce HLA-G or non-cleavable HLA-G and/or a cytokine signaling complex comprising the partial or full peptide of the cell surface expressed exogenous cytokine and/or receptor thereof. In various embodiments, the genomic engineering comprises targeted editing. In certain embodiments, the targeted editing comprises deletion, insertion, or in/del, and wherein the targeted editing is carried out by CRISPR, ZFN, TALEN, homing nuclease, homology recombination, or any other functional variation of these methods.

In yet another aspect, the invention provides a recombinant receptor, comprising: (a) a partial or full peptide of the extracellular domain (ECD) of a signaling receptor comprising transforming growth factor beta receptor (TGFβR), programmed cell death 1 (PD1), CTLA4, IL10R, IL4R, or any combination thereof; and (b) a partial or full peptide of the intracellular domain (ICD) of a cytokine receptor comprising IL2Rβ, IL12Rβ, IL18Rβ, IL21R, or any combination thereof. In various embodiments, the signaling receptor comprises TGFβR2, wherein the recombinant receptor is a TGFβ signaling redirector receptor (TGFβ-SRR), and wherein the cytokine receptor providing the intracellular domain (ICD) is (a) IL2Rβ, thereby forming a TGFβR2-IL2Rβ signaling redirector receptor; or (b) IL12Rβ, thereby forming a TGFβR2-IL12Rβ signaling redirector receptor; or (c) IL18Rβ, thereby forming a TGFβR2-IL18Rβ signaling redirector receptor; or (d) IL21R, thereby forming a TGFβR2-IL21R signaling redirector receptor. In some embodiments, (a) the intracellular domain (ICD) of IL2Rβ comprises an amino acid sequence represented by SEQ ID NO: 2 (NCRNTGPWLKKVLKCNTPDPSKFFSQLSSEHGGDVQKWLSSPFPSSSFSPGGLAPEISPL EVLERDKVTQLLLQQDKVPEPASLSSNHSLTSCFTNQGYFFFHLPDALEIEACQVYFTYD PYSEEDPDEGVAGAPTGSSPQPLQPLSGEDDAYCTFPSRDDLLLFSPSLLGGPSPPSTAPGG SGAGEERMPPSLQERVPRDWDPQPLGPPTPGVPDLVDFQPPPELVLREAGEEVPDAGPRE GVSFPWSRPPGQGEFRALNARLPLNTDAYLSLQELQGQDPTHLV); or (b) the intracellular domain (ICD) of IL12Rβ comprises an amino acid sequence represented by (i) SEQ ID NO: 3 (HYFQQKVFV LLAALRPQWCSREIPDPANSTCAKKYPIAEEKTQLPLDRLLIDWPTPEDPE PLVISEVLHQVTPVFRHPPCSNWPQREKGIQGHQASEKDMMHSASSPPPPRALQAESRQL VDLYKVLESRGSDPKPENPACPWTVLPAGDLPTHDGYLPSNIDDLPSHEAPLADSLEELE PQHISLSVFPSSSLHPLTFSCGDKLTLDQLKMRCDSLML), or (ii) SEQ ID NO: 4 (SDPKPENPACPWTVLPAGDLPTHDGYLPSNIDDLPSHEAPLADSLEELEPQ); or (c) the intracellular domain (ICD) of IL18Rβ comprises an amino acid sequence represented by SEQ ID NO: 5 (YRVDLVLFYRHLTRRDETLTDGKTYDAFVSYLKECRPENGEEHTFAVEILPRVLEKHFGY KLCIFERDVVPGGAVVDEIHSLIEKSRRLIIVLSKSYMSNEVRYELESGLHEALVERKIKIIL IEFTPVTDFTFLPQSLKLLKSHRVLKWKADKSLSYNSRFWKNLLYLMPAKTVKPGRDEP EVLPVLSES); or (d) the intracellular domain (ICD) of IL21Rβ comprises an amino acid sequence represented by SEQ ID NO: 6 (SLKTHPLWRLWKKIWAVPSPERFFMPLYKGCSGDFKKWVGAPFTGSSLELGPWSPEVPS TLEVYSCHPPRSPAKRLQLTELQEPAELVESDGVPKPSFWPTAQNSGGSAYSEERDRPYG LVSIDTVTVLDAEGPCTWPCSCEDDGYPALDLDAGLEPSPGLEDPLLDAGTTVLSCGCVS AGSPGLGGPLGSLLDRLKPPLADGEDWAGGLPWGGRSPGGVSESEAGSPLAGLDMDTF DSGFVGSDCSSPVECDFTSPGDEGPPRSYLRQWVVIPPPLSSPGPQAS), and (e) the extracellular domain (ECD) of TGFβR comprises an amino acid sequence represented by SEQ ID NO: 1 (TIPPHVQKSVNNDMIVTDNNGAVKFPQLCKFCDVRFSTCDNQKSCMSNCSITSICEKPQ EVCVAVWRKNDENITLETVCHDPKLPYHDFILEDAASPKCIMKEKKKPGETFFMCSCSSD ECNDNIIFSEEYNTSNPDLLLVIFQ). In one embodiment, the TGFβR2-IL12Rβ signaling redirector receptor comprises an amino acid sequence having a sequence identity of at least 80%, 85%, 90%, 95%, or 97%, 98%, or 99% to a sequence represented by SEQ ID NO: 7, wherein an amino acid sequence represented by SEQ ID NO: 8 comprised in SEQ ID NO: 7 is variable.

In yet another aspect, the invention provides a polynucleotide encodement the recombinant receptor provided herein.

In yet another aspect, the invention provides a method of sensitizing tumor cells, wherein the method comprises contacting the tumor cells with the recombinant receptor provided herein, thereby inhibiting or reducing the signaling of TGFβ expressed by, or in the environment of, the tumor cells. In various embodiments, recombinant receptor is expressed by immune effector cells engineered with a polynucleotide encoding the recombinant receptor as provided herein. In some embodiments, the immune effector cells are administered to a subject in need of tumor sensitizing. In some embodiments, the immune effector cells are derived from iPSCs comprising a polynucleotide encoding the recombinant receptor. In some embodiments, the immune effector cells are NK cells, T cells, or a combination thereof. In some embodiments, the immune effector cells are autologous or allogeneic to the subject in need of tumor sensitizing. In particular embodiments, the tumor cells are of a solid tumor as provided herein.

In yet another aspect, the invention provides a method of reducing or preventing tumor microenvironment suppression against effector cells in an adoptive cell therapy provided to a subject in need thereof, the method comprising administering the effector cells to the subject, wherein the effector cells comprise the derivative cell or population thereof as described herein. In various embodiments, the effector cells comprise alloreactive immune cells comprising primary T cells, B cells, and/or NK cells. In various embodiments of the method of reducing or preventing tumor microenvironment suppression against effector cells in an adoptive cell therapy, the method further comprises administering one or more therapeutic agents to the subject. In some embodiments, the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In particular embodiments, (i) the checkpoint inhibitor comprises: (a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO. LAIR-1, MICA/B, NR4A2, MAFB, OCT-2, Rara (retinoic acid receptor alpha), TLR3, VISTA, NKG2A/HLA-E, or inhibitory KIR; (b) one or more of atezolizumab, avelumab, durvalumab, ipilimumab, IPH4102, IPH43, IPH33, lirimumab, monalizumab, nivolumab, pembrolizumab, and their derivatives or functional equivalents; or (c) at least one of atezolizumab, nivolumab, and pembrolizumab; or (ii) the one or more therapeutic agents comprise one or more of venetoclax, azacitidine, and pomalidomide.

In another aspect, the invention provides a method of producing a clonal master engineered iPSC line using CRISPR, ZFN, or TALEN mediated editing of clonal iPSCs, wherein the editing comprises a knock-in of a polynucleotide encoding a recombinant receptor as provided herein, and optionally one or more of: (a) an exogenous polynucleotide encoding a CAR (chimeric antigen receptor); (b) an exogenous polynucleotide encoding a CD16 or a variant thereof; (c) CD38 knockout; and (d) an exogenous polynucleotide encoding a cytokine signaling complex comprising a partial or full peptide of a cell surface expressed exogenous cytokine and/or a receptor thereof, thereby producing the engineered iPSCs.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIGS. 1A and 1B are graphic representations showing that tumor growth inhibition of standard CAR19-iT cells (FIG. 1A) and engineered CAR19-iT cells (FIG. 1B) co-cultured with Nalm6 target cells, is enhanced in the presence of select cytokines.

FIGS. 2A-2D are graphic representations showing that addition of select cytokines enhances iNK function. The effect of adding IL-12 (FIG. 2A), IL-21 (FIG. 2B), IL-2 (FIG. 2C), and IL-18 (FIG. 2D) was tested in standard iNKs (left graphs) or CAR-iNKs (right graphs) by co-culturing effector cells with target cells and measuring % cytolysis over time.

FIGS. 3A and 3B are graphic representations showing that addition of select cytokines to a co-culture of CAR-iNK cells and tumor cells leads to increased CAR-iNK cell persistence in the presence of TGFβ in vitro. CAR frequency (FIG. 3A) and CAR counts (FIG. 3B) were measured by flow cytometry.

FIGS. 4A and 4B are graphic representations showing that treatment of primary T cells expressing TGFβR2-IL21R with recombinant TGFβ leads to a dose dependent increase in pSTAT3 and pSTAT5 signaling in vitro.

FIG. 5A shows inducible cytokine signaling via TGFβ. FIG. 5B shows improved functionality of TGFβ redirector receptors over dnTGFβR2 expression thereof.

FIGS. 6A and 6B are a series of graphic representations showing Thy1.2 and TGFβR2 expression 24 hours post transduction in CAR19-iT cells by flow cytometry.

FIGS. 7A-7C are graphic representations showing that redirecting TGFβ signaling in CAR19-iT cells leads to increased tumor growth control in vitro. CAR19-iT cells were transduced with lentivirus particles encoding TGFβR2-IL2Rb (FIG. 6A), TGFβR2-IL12Rβ (FIG. 6B), or TGFβR2-IL21R (FIG. 6C) to compare the effect of TGFβ redirection on tumor growth control.

FIGS. 8A-8C are graphic representations showing that redirecting TGFβ signaling in CAR19-iT cells does not lead to increased CAR persistence in vitro.

FIG. 9A shows a schematic of an exemplary strategy for generating and testing a population of CAR-iT cells expressing a signal redirector receptor. FIG. 9B shows flow cytometric detection of the TGFβ redirector receptors expressed by CAR-iT cells. FIG. 9C shows that CAR-iT cells expressing a TGFβ redirector receptor can be successfully differentiated from a bulk-engineered iPSC population.

FIG. 10 (parts A and B) shows that CAR-iT cells expressing a TGFβ redirector receptor maintain effector function in the presence of TGFβ.

FIG. 11A shows that expression of a TGFβR2-IL18R redirector receptor transgene is maintained in CAR-iT cells after multiple rounds of stimulation with target cells. FIG. 11B shows that CAR-iT cells with the TGFβR2-IL18R redirector receptor transgene exhibited enhanced activation profiles compared to control CAR-iT cells.

FIG. 12 shows enhanced cytokine secretion profiles in CAR-iT cells with a TGFβR2-IL18R redirector receptor in a serial stimulation assay.

FIG. 13 shows CAR-iT cells with the TGFβR2-IL18R Redirector Receptor exhibit improved expansion in the serial stim assay with or without TGFβ.

FIG. 14 shows detected surface expression of the indicated NK markers on iNK cells that were differentiated from iPSCs comprising a TGFβR2-trIL12Rβ redirector receptor.

FIG. 15A shows that the TGFβR2-trIL12Rβ iNK cells exhibit robust persistence and expansion even in the presence of TGFβ, and FIG. 15B shows increased proportion of TGFβR2-trIL12Rβ expressing iNK cells with phenotypic and activation markers as compared with iNK cells expressing dnTGFβR2.

FIG. 16A shows that the TGFβR2-trIL12Rβ iNK cells exhibit robust innate killing capacity toward target cells compared to iNK cells expressing dnTGFβR2 and parental iNK cells. FIG. 16B shows that the TGFβR2-trIL12Rβ iNK cells exhibit increased expansion compared to iNK cells expressing dnTGFβR2 and parental iNK cells over three rounds of restimulation with target cells. FIG. 16C shows an increased proportion of TGFβR2-trIL12Rβ expressing iNK cells with phenotypic and activation markers as compared with iNK cells expressing dnTGFβR2 and parental iNK cells (control).

FIG. 17 shows quantification of cytokines TNF, IFNγ, and GMCSF secreted by TGFβR2-trIL12Rβ expressing iNKs cocultured with MDA-MB-231 cells compared to cytokine secretion of parental iNKs against the same targets.

FIGS. 18A and 18B show that TGFβR2-trIL12Rβ iNKs exhibit enhanced cytolysis of target breast cancer cells with ADCC both in the presence and absence of TGFβ, and TGFβR2-trIL12Rβ iNKs exhibit resistance to TGFβ mediated suppression (FIG. 18B).

FIG. 19A shows enhanced persistence of TGFβR2-trIL12Rβ expressing iNKs compared to iNKs expressing dnTGFβR2. FIG. 19B shows that the TGFβR2-trIL12Rβ construct is more stable and highly expressed on the surface of iNK cells in the presence of TGFβ.

FIGS. 20A and 20B show that TGFβR2-trIL12Rβ iNKs exhibit superior antitumor ADCC activity in a second round of co-culture with target cells in the presence of TGFβ, compared to parental iNKs and dnTGFβR2 iNKs.

FIG. 21 TGFβ-SRR iNKs exhibit enhanced ADCC cytolysis across various solid cancer cell lines in the presence of suppressive signaling of TGFβ.

### DETAILED DESCRIPTION OF THE INVENTION

Genomic modification of iPSCs (induced pluripotent stem cells) includes polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cells), T cell progenitor cells, NK cell progenitor cells, T cells, NKT cells, NK cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example. “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or +1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition at a functionally inert, low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains.” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refer to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, refers to stem cells that are produced in vitro from differentiated adult, neonatal or fetal cells that have been induced or changed, i.e., reprogrammed into cells capable of differentiating into tissues of all three germ or dermal layers: mesoderm, endoderm, and ectoderm. In some embodiments, the reprogramming process uses reprogramming factors and/or small molecule chemical driven methods. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta (i.e., are not totipotent).

As used herein, the term “multipotent stem cell” refers to a cell that has the developmental potential to differentiate into cells of one or more germ layers (i.e., ectoderm, mesoderm and endoderm), but not all three. Thus, a multipotent cell can also be termed a “partially differentiated cell.” Multipotent cells are known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to the state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343. TG30, CD9, CD29, CD133/prominin, CD140a, CD56, CD73, CD90, CD105, OCT4, NANOG, SOX2, CD30 and/or CD50; (iv) the ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed state of pluripotency. Under standard pluripotent cell culture conditions such cells remain in the primed state unless the exogenous transgene expression is maintained, wherein characteristics of the ground state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media.” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells. NK lineage cells and B lineage cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restncted manner. AT cell can be any T cell, such as a cultured T cell, e.g., a primary T cell, or a T cell from a cultured T cell line, e.g., Jurkat, SupTI, etc., or a T cell obtained from a mammal. The T cell can be a CD3′ cell. The T cell can be any type of T cell and can be of any developmental stage, including but not limited to, CD4⁺/CD8⁺ double positive T cells, CD4-helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (Q T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”.

“CD4⁺ T cells” refers to a subset of T cells that express CD4 on their surface and are associated with cell-mediated immune response. They are characterized by secretion profiles following stimulation, which may include secretion of cytokines such as IFN-gamma, TNF-alpha. IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class 1-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class 1-restricted interactions.

As used herein, the term “NK cell” or “Natural Killer cell” refer to a subset of peripheral blood lymphocytes defined by the expression of CD56 or CD16 and the absence of the T cell receptor (CD3). An NK cell can be any NK cell, such as a cultured NK cell, e.g., a primary NK cell, or an NK cell from a cultured or expanded NK cell or a cell-line NK cell, e.g., NK-92, or an NK cell obtained from a mammal that is healthy or with a disease condition. As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56′ NK cells comprise expression of CD16, NKG2C, CD57, NKG2D, NCR ligands, NKp30, NKp40, NKp46, activating and inhibitory KIRs, NKG2A and/or DNAM-1. CD56′ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”.

As used herein, the term “NKT cells” or “natural killer T cells” or “NKT lineage cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified by the expression of one or more of the following markers: TCR Va24-Ja18, Vb11, CD1d, CD3, CD4, CD8, aGalCer, CD161 and CD56.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as “encoding” the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Thus, the term “vector” comprises the construct to be delivered. A vector can be a linear or a circular molecule. A vector can be integrating or non-integrating. The major types of vectors include, but are not limited to, plasmids, episomal vectors, viral vectors, cosmids, and artificial chromosomes. Viral vectors include, but are not limited to, adenovirus vectors, adeno-associated virus vectors, retrovirus vectors, lentivirus vectors, Sendai virus vectors, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or is non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences. cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. “Polynucleotide” also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” refers to each separate polypeptide chain of a protein complex, where each separate polypeptide chain can form a stable folded structure by itself. Many protein molecules are composed of more than one subunit, where the amino acid sequences can either be identical for each subunit, or similar, or completely different. For example, CD3 complex is composed of CD3α, CD3ε, CD3δ, CD3γ, and CD3ζ subunits, which form the CD3ε/CD3γ, CD3ε/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK cells or derivative T cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSCs using genomic editing. In the aspect of a source cell obtained from a specifically selected donor, disease or treatment context, the genetic imprint contributing to preferential therapeutic attributes may include any context-specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof: resistance to tumor microenvironment; induction of bystander immune cells and immune modulations; improved on-target specificity with reduced off-tumor effect; and/or resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, a synthetic cell possesses one or more non-native cell functions when compared to its closest counterpart primary cell.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression: HLA presentation or lack thereof; resistance to tumor microenvironment; induction of bystander immune cells and immune modulations; improved on-target specificity with reduced off-tumor effect; and/or resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil) and a tumor cell, and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and a specific target cell (e.g., a tumor cell) independent of the effector cells' natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some cases, and to kill two or more types of tumors in other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch protein could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate, relieve, alleviate, reverse or lessen the severity of a disease. A pharmaceutically active protein also has prophylactic properties and is used to prevent the onset of a disease or to lessen the severity of such disease or pathological condition when it does emerge. “Pharmaceutically active proteins” include an entire protein or peptide or pharmaceutically active fragments thereof. The term also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Examples of signal transduction pathways are known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes, but is not limited to, i) antigen specificity as it relates to a unique chimeric antigen receptor (CAR) or T cell receptor (TCR), ii) engager specificity as it relates to monoclonal antibodies or bispecific engagers, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogeneic lymphocytes, whether the immune cells are isolated from a human donor or effector cells obtained from in vitro differentiation of a pluripotent cell; whether they are genetically modified or not; or whether they are primary donor cells or cells that have been passaged, expanded, or immortalized, ex vivo, after isolation from a donor. As used herein, “lymphodepletion” and “lympho-conditioning” are used interchangeably to refer to the destruction of lymphocytes and T cells, typically prior to immunotherapy. The purpose of lympho-conditioning prior to the administration of an adoptive cell therapy is to promote homeostatic proliferation of effector cells as well as to eliminate regulatory immune cells and other competing elements of the immune system that compete for homeostatic cytokines. Thus, lympho-conditioning is typically accomplished by administering one or more chemotherapeutic agents to the subject prior to a first dose of the adoptive cell therapy. In various embodiments, lympho-conditioning precedes the first dose of the adoptive cell therapy by a few hours to a few days. Exemplary chemotherapeutic agents useful for lympho-conditioning include, but are not limited to, cyclophosphamide (CY), fludarabine (FLU), and those described below. However, a sufficient lymphodepletion through anti-CD38 mAb could provide an alternative conditioning process for the present iNK cell therapy, without or with minimal need of a CY/FLU-based lympho-conditioning procedure, as further described herein.

As used herein, “homing” or “trafficking” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of a cell to a target site.

A “therapeutically sufficient amount”, as used herein, includes within its meaning a non-toxic, but sufficient and/or effective amount of a particular therapeutic agent and/or pharmaceutical composition to which it is referring to provide a desired therapeutic effect. The exact amount required will vary from subject to subject, depending on factors such as the patient's general health, the patient's age and the stage and severity of the condition being treated. In particular embodiments, a “therapeutically sufficient amount” is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to the differentiation cues within the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture media maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation to initiate differentiation. Because monolayer culturing does not mimic embryo development such as is the case with EB formation, differentiation towards specific lineages is deemed to be minimal as compared to all three germ layer differentiation in EB formation.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated w % ben being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level-successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, HLA class 11 deficient, or both, refers to cells that either lack, or no longer maintain, or have a reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related, but not limited to improved differentiation potential, antigen targeting, antigen presentation, antibody recognition, persistence, immune evasion, resistance to suppression, proliferation, costimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11 b, NOTCH, RUNX1, IL15, 4-1BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” is used herein in the broadest sense and refers generally to an immune-response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobodyt@ fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells. T and B cells) and the signaling properties of each receptor. Fc-gamma receptors (FcγR) include several members, FcγRI (CD64), FcγRIIA (CD32), FcγRIIB (CD32), FcγRIIIA (CD16a), and FcγRITIB (CD16b), which differ in their antibody affinities due to their different molecular structures.

“Chimeric Receptor” is a general term used to describe an engineered, artificial, or a hybrid receptor protein molecule that is made to comprise two or more portions of amino acid sequences that are originated from at least two different proteins. The chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), as well as fusions of two or more receptors.

“Chimeric Fc Receptor,” abbreviated as CFcR, is a term used to describe engineered Fc receptors having their native transmembrane and/or intracellular signaling domains modified or replaced with non-native transmembrane and/or intracellular signaling domains. In some embodiments of the chimeric Fc receptor, in addition to having one of, or both of, the transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR), which contains an antigen binding domain to a target antigen, the chimeric Fc receptor binds to an Fc fragment, or the Fc region of an antibody, or the Fc region comprised in an engager or a binding molecule and activates the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16-based CFcR, the extracellular domain of CD64 or the high-affinity variants of CD16 (F176V, for example) can be incorporated. In some embodiments of the CFcR where a high affinity CD16 extracellular domain is involved, the proteolytic cleavage site comprising a serine at position 197 is eliminated or is replaced such at the extracellular domain of the receptor is non-cleavable, i.e., not subject to shedding, thereby obtaining a hnCD16-based CFcR.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

“T cell receptor,” abbreviated as “TCR,” generally refers to a protein complex found on the surface of a T cell and is responsible for recognizing fragments of antigen peptides bound to major histocompatibility complex (MHC) molecules. Binding of a TCR to an antigen peptide initiates TCR-CD3 intracellular activation, recruitment of numerous signaling molecules, and branching and integrating signaling pathways, leading to mobilization of transcription factors that are important for gene expression and typical T cell growth and function acquisition. A typical TCR comprises two highly variable protein chains (α and β), with each chain comprising a constant region proximal to the cell membrane and a variable region (i.e., binding domain) that binds to the peptide/MHC.

1. Cells and Compositions Useful for Adoptive Cell Therapies with Enhanced Properties

Provided herein is a strategy to systematically engineer the regulatory circuitry of a clonal iPSC without impacting the differentiation potency and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modified activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and the need for reconfiguration of differentiation protocols enabling phenotypic and/or functional change in the cell. The present application has shown that the one or more selected genomic modifications as provided herein does not negatively impact iPSC differentiation potency, and the functional effector cells derived from the engineered iPSC have enhanced and/or acquired therapeutic properties attributable to the individual or combined genomic modifications retained in the effector cells following the iPSC differentiation.

### 1. Exogenously Introduced Cytokine Signaling Complex

By avoiding systemic high-dose administration of clinically relevant cytokines, the risk of dose-limiting toxicities due to such a practice is reduced while cytokine mediated cell autonomy is being established. To achieve lymphocyte autonomy without the need to additionally administer soluble cytokines, according to various embodiments, provided is a cytokine signaling complex that comprises an intracellular domain comprising a partial or full peptide of an intracellular domain (ICD) of a cytokine receptor. In some embodiments, the cytokine signaling complex comprises an extracellular domain (ECD) comprising a partial or full peptide of one or more cytokines including, but not limited to, IL2, IL4, IL6, IL7, IL9, IL10, IL11, IL12, IL15, IL18, IL21, and/or their respective receptors. Such a cytokine signaling complex may be introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling (signaling complex) are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through an expression construct carried by a retrovirus, Sendai virus, an adenovirus, an episome, mini-circle, or RNAs including mRNA.

Various construct designs for introducing a protein complex for signaling of one, two, or more cytokines including, but not limited to, IL2, IL4, IL6, IL7, IL9, IL10, IL11, IL12, IL15, IL18 and IL21, into the cell are provided herein. For example, in embodiments where the cytokine signaling complex is for IL15, the transmembrane (TM) domain can be native to the IL15 receptor or may be modified or replaced with the transmembrane domain of any other membrane bound proteins. In various embodiments, the cytokine signaling complex comprises an IL15 receptor fusion (IL15RF) comprising a full or partial length of IL15 and a full or partial length of IL15 receptor (IL15R). In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL5 as well as ensuring that IL15 is membrane-bound. In other embodiments, IL15Rα with a truncated intracellular domain is fused to IL15 at the C-terminus through a linker, mimicking trans-presentation of IL15, maintaining membrane-bound IL15, and additionally eliminating cis-presentation and/or any other potential signal transduction pathways mediated by a normal IL15R through its intracellular domain. In other embodiments, IL15Rα is fused to IL15 without an intracellular domain (IL15Δ), as described in International Pub. Nos. WO 2019/191495 and WO 2019/126748, the entire disclosure of each of which is incorporated herein by reference.

In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the trans-membrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of IL15 is retained.

In other embodiments, a native or modified IL15Rβ is fused to IL15 at the C-terminus through a linker, enabling constitutive signaling and maintaining IL15 membrane-bound and trans-representation. In other embodiments, a native or modified common receptor γC is fused to IL15 at the C-terminus through a linker for constitutive signaling and membrane bound trans-presentation of the cytokine. The common receptor γC is also called the common gamma chain or CD132, which is also known as IL2 receptor subunit gamma or IL2RG. γC is a cytokine receptor subunit that is common to the receptor complexes for many interleukin receptors, including, but not limited to, IL2, IL4, IL7, IL9, IL15 and IL21 receptors. In other embodiments, engineered IL15Rβ that forms a homodimer in the absence of IL15 is useful for producing constitutive signaling of the cytokine.

In other various embodiments, the cytokine signaling complex comprises an IL7 receptor fusion (IL7RF) comprising a full or partial length of IL7 and a full or partial length of IL7 receptor. The transmembrane (TM) domain can be native to the IL7 receptor or may be modified or replaced with a transmembrane domain of any other membrane bound proteins. In one embodiment, a native (or wildtype) or modified IL7R is fused to IL7 at the C-terminus through a linker, enabling constitutive signaling and maintaining membrane-bound IL7.

In another embodiment, a native or modified common receptor γC is fused to IL7 at the C-terminus through a linker for constitutive and membrane-bound cytokine signaling complex. In addition, engineered IL7R that forms a homodimer in the absence of IL7 is useful for producing constitutive signaling of the cytokine as well.

In some other embodiments of a cytokine signaling complex comprising a partial or full peptide of an intracellular domain (ICD) of a cytokine receptor, the cytokine signaling complex comprises a partial or full peptide of an extracellular domain (ECD) of a signaling receptor, and an intracellular domain, or a fragment thereof, of a cytokine receptor, wherein the signaling receptor and the cytokine receptor are different molecules. In some embodiments, the cytokine signaling complex is a signaling redirector receptor. In general, a “signaling (or signal) redirector receptor” or “SRR” redirects the signaling of an extracellular domain from one receptor (e.g., a TGFβ receptor) through an intracellular domain from a different receptor (e.g., a cytokine receptor) by joining the extracellular and intracellular domains of the different receptors. Exemplary signaling receptors that provide extracellular domains include, but are not limited to, transforming growth factor beta receptor (TGFβR), programmed cell death 1 (PD1), CTLA4, IL10R, IL4R or any combination thereof. In the context of TGFβR, the signaling redirector receptor may be referred to as a “TGFβR redirector” or “TGFβR redirector receptor” or “TGFβ signal redirector receptor” or “TGFβ-SRR” throughout this application. In various embodiments, the signaling redirector receptor comprises: (i) an extracellular domain, or a fragment thereof, of a signaling receptor comprising transforming growth factor beta receptor (TGFβR), programmed cell death 1 (PD1). CTLA4, IL10R, IL4R, or any combination thereof; and (ii) an intracellular domain, or a fragment thereof, of a cytokine receptor comprising IL2R, IL12R, IL18R, IL21R. or any combination thereof.

In some embodiments, the extracellular domain (ECD) of TGFβR comprises an amino acid sequence represented by an amino acid sequence having at least about 85%, about 90%, about 95%, about 96%, about 97%, about 98%, or about 99% identity to SEQ ID NO: 1. In some embodiments, the intracellular domain (ICD) of IL2Rβ comprises an amino acid sequence represented by an amino acid sequence having at least about 85%, about 90%, about 95%, about 96%, about 97%, about 98%, or about 99% identity to SEQ ID NO: 2. In some embodiments, the intracellular domain (ICD) of IL12Rβ comprises an amino acid sequence represented by an amino acid sequence having at least about 85%, about 90%, about 95%, about 96%, about 97%, about 98%, or about 99% identity to SEQ ID NO: 3 or 4. In some embodiments, the intracellular domain (ICD) of IL18Rβ comprises an amino acid sequence represented by an amino acid sequence having at least about 85%, about 90%, about 95%, about 96%, about 97%, about 98%, or about 99% identity to SEQ ID NO: 5. In some embodiments, the intracellular domain (ICD) of IL21Rβ comprises an amino acid sequence represented by an amino acid sequence having at least about 85%, about 90%, about 95%, about 96%, about 97%, about 98%, or about 99% identity to SEQ ID NO: 6. As used herein and throughout the application, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm recognized in the art.

SEQ ID NO: 1
TIPPHVQKSVNNDMIVTDNNGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDECNDNIIFSEEYNTSNPDLLL
VIFQ
(ECD of TGFβR)
SEQ ID NO: 16
FLDSPDRPWN PPTFSPALLV VTEGDNATFT CSFSNTSESF VLNWYRMSPS NQTDKLAAFP
EDRSQPGQDC RFRVTQLPNG RDFHMSVVRA RRNDSGTYLC GAISLAPKAQ IKESLRAELR
VTERRAEVPT AHPSPSPRPA GQFQTLV
(ECD of PD1)
SEQ ID NO: 17
KAMHVAQPAV VLASSRGIAS FVCEYASPGK ATEVRVTVLR QADSQVTEVC AATYMMGNEL
TFLDDSICTG TSSGNQVNLT IQGLRAMDTG LYICKVELMY PPPYYLGIGN GTQIYVIDPE
PCPDSD
(ECD of CTLA4)
SEQ ID NO: 18
HGTELPSPPS VWFEAEFFHH ILHWTPIPNQ SESTCYEVAL LRYGIESWNS ISNCSQTLSY
DLTAVTLDLY HSNGYRARVR AVDGSRHSNW TVTNTRFSVD EVTLTVGSVN LEIHNGFILG
KIQLPRPKMA PANDTYESIF SHFREYEIAI RKVPGNFTFT HKKVKHENFS LLTSGEVGEF
CVQVKPSVAS RSNKGMWSKE ECISLTRQYF TVTN
(ECD of IL10R)
SEQ ID NO: 19
MKVLQEPTCV SDYMSISTCE WKMNGPTNCS TELRLLYQLV FLLSEAHTCI PENNGGAGCV
CHLLMDDVVS ADNYTLDLWA GQQLLWKGSF KPSEHVKPRA PGNLTVHTNV SDTLLLTWSN
PYPPDNYLYN HLTYAVNIWS ENDPADFRIY NVTYLEPSLR IAASTLKSGI SYRARVRAWA
QCYNTTWSEW SPSTKWHNSY REPFEQH
(ECD of IL4R)
SEQ ID NO: 2
NCRNTGPWLKKVLKCNTPDPSKFFSQLSSEHGGDVQKWLSSPFPSSSFSPGGLAPEISPLEVLERDKVTQ
LLLQQDKVPEPASLSSNHSLTSCFTNQGYFFFHLPDALEIEACQVYFTYDPYSEEDPDEGVAGAPTGSSP
QPLQPLSGEDDAYCTFPSRDDLLLFSPSLLGGPSPPSTAPGGSGAGEERMPPSLQERVPRDWDPQPLGPP
TPGVPDLVDFQPPPELVLREAGEEVPDAGPREGVSFPWSRPPGQGEFRALNARLPLNTDAYLSLQELQGQ
DPTHLV
(ICD of IL2Rβ)
SEQ ID NO: 3
HYFQQKVFVLLAALRPQWCSREIPDPANSTCAKKYPIAEEKTQLPLDRLLIDWPTPEDPEPLVISEVLHQ
VTPVFRHPPCSNWPQREKGIQGHQASEKDMMHSASSPPPPRALQAESRQLVDLYKVLESRGSDPKPENPA
CPWTVLPAGDLPTHDGYLPSNIDDLPSHEAPLADSLEELEPQHISLSVFPSSSLHPLTFSCGDKLTLDQL
KMRCDSLML
(ICD of IL2Rβ)
SEQ ID NO: 4
SDPKPENPACPWTVLPAGDLPTHDGYLPSNIDDLPSHEAPLADSLEELEPQ
(ICD of IL2Rβ; truncated)
SEQ ID NO: 5
YRVDLVLFYRHLTRRDETLTDGKTYDAFVSYLKECRPENGEEHTFAVEILPRVLEKHFGYKLCIFERDVV
PGGAVVDEIHSLIEKSRRLIIVLSKSYMSNEVRYELESGLHEALVERKIKIILIEFTPVTDFTFLPQSLK
LLKSHRVLKWKADKSLSYNSRFWKNLLYLMPAKTVKPGRDEPEVLPVLSES
(ICD of IL8Rβ)
SEQ ID NO: 6
SLKTHPLWRLWKKIWAVPSPERFFMPLYKGCSGDFKKWVGAPFTGSSLELGPWSPEVPSTLEVYSCHPPR
SPAKRLQLTELQEPAELVESDGVPKPSFWPTAQNSGGSAYSEERDRPYGLVSIDTVTVLDAEGPCTWPCS
CEDDGYPALDLDAGLEPSPGLEDPLLDAGTTVLSCGCVSAGSPGLGGPLGSLLDRLKPPLADGEDWAGGL
PWGGRSPGGVSESEAGSPLAGLDMDTFDSGFVGSDCSSPVECDFTSPGDEGPPRSYLRQWVVIPPPLSSP
GPQAS
(ICD of IL21Rβ)

In some embodiments of a cytokine signaling complex (e.g., a signaling redirector receptor, or “SRR”) comprising an intracellular domain or a fragment thereof of one or more cytokine receptors and an extracellular domain or a fragment thereof of a signaling receptor, the cytokine signaling complex further comprises a transmembrane domain (TM). In various embodiments, the transmembrane (TM) domain of the cytokine signaling complex can: (i) originate from the same molecule providing the intracellular domain, (ii) originate from the same molecule providing the extracellular domain, or (iii) may be modified or replaced with a transmembrane domain of any other membrane bound proteins.

In some embodiments, the cytokine receptor providing an intracellular domain or a fragment thereof of the cytokine signaling complex comprises at least one of IL2R, IL4R, IL6R, IL7R, IL9R, IL10R, IL1R. IL12R, IL15R, IL18R and IL21R In some embodiments, the signaling receptor providing an extracellular domain or a fragment thereof of the cytokine signaling complex may be a growth factor receptor, a chemokine receptor, or a hormone receptor. In some embodiments, the cytokine signaling complex comprises an extracellular domain or a fragment thereof of transforming growth factor beta receptor (TGFβR), programmed cell death 1 (PD1), CTLA4, IL10R, IL4R, or any combination thereof.

In one embodiment, the cytokine signaling complex comprises an extracellular domain or a fragment thereof of the signaling receptor TGFβR (e.g., as in a TGFβ signaling redirector receptor). Transforming growth factor beta (TGFβ) is an immuno-suppressive cytokine commonly present in the tumor microenvironment (TME) that creates considerable challenges for the treatment of solid tumors. Chimeric TGFβ signal redirector receptors (TGFβ-SRR), in accordance with embodiments described herein, were shown to block the TGFβ-mediated repressive signaling and redirect the signal to potentiate effector cell function and improve cell fitness to improve therapeutic efficacy in solid tumors. In some embodiments, the signaling receptor comprises an extracellular domain or a fragment thereof of TGFβR and an intracellular domain or a fragment thereof of the cytokine receptor IL2Rβ, thereby forming a TGFβR2-IL2R signaling redirector receptor. In some embodiments, the signaling receptor comprises an extracellular domain or a fragment thereof of TGFβ R and an intracellular domain or a fragment thereof of the cytokine receptor IL12R$4, thereby forming a TGFβR2-IL12Rβ signaling redirector receptor. In some embodiments, the signaling receptor comprises an extracellular domain or a fragment thereof of TGFβR and an intracellular domain or a fragment thereof of the cytokine receptor IL18Rβ, thereby forming a TGFβR2-IL18Rβ signaling redirector receptor. In some embodiments, the signaling receptor comprises an extracellular domain or a fragment thereof of TGFβR and an intracellular domain or a fragment thereof of the cytokine receptor IL21R, thereby forming a TGFβR2-IL21R signaling redirector receptor. In some embodiments, the TGFβR2-IL12R13 signaling redirector receptor comprises an amino acid sequence having sequence identity of at least 80%, 85%, 90%, 95%, or 97%, 98%, or 99% to a sequence represented by SEQ ID NO: 7 (termed specifically as TGFβR2-trIL12Rβ throughout the application). In some embodiments, the transmembrane domain sequence represented by SEQ ID NO: 8 comprised within SEQ ID NO: 7 may vary in sequence or length or may be replaced with a transmembrane domain of another transmembrane protein.

SEQ ID NO: 7
TIPPHVQKSVNNDMIVTDNNGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDECNDNIIFSEEYNTSNPDLLL
VIFQVTGISLLPPLGVAISVIIIFYCYRVNSDPKPENPACPWTVLPAGDLPTHDGYLPSNIDDLPSHEAP
LADSLEELEPQ
(TGFβR2-trIL12Rβ)
SEQ ID NO: 8
VTGISLLPPLGVAISVIIIFYCYRVN
(variable portion of TGFβR2-trIL12Rβ)

As such, in various embodiments, a cytokine signaling complex as provided herein may be introduced to iPSC using one or more of the construct designs described above, and to its derivative cells upon iPSC differentiation. In addition to an induced pluripotent cell (iPSC), a clonal iPSC, a clonal iPS cell line, or iPSC derived cells comprising at least one engineered modality as disclosed herein are provided. In some embodiments, the present invention provides iPSCs and derivative cells therefrom comprising a signaling redirector receptor as described herein (“SRR” in Table 1), wherein the cells, such as derivative T and NK cells, are effector cells useful for overcoming or reducing tumor microenvironment suppression associated with a solid tumor, and wherein the signaling redirector receptor comprises a partial or full peptide of an extracellular domain (ECD) of a signaling receptor, and an intracellular domain (ICD) comprising a partial or full peptide of a cytokine receptor, wherein the signaling receptor and the cytokine receptor are different molecules. In some embodiments, the iPSC and derivative cells thereof optionally further comprise an exogenous cytokine signaling complex comprising a polynucleotide encoding a full or partial peptide of a cytokine and/or a full or partial peptide of its receptor (“IL” in Table 1) to enable further cytokine signaling contributing to cell survival, persistence and/or expansion. In some embodiments, the iPSC and derivative cells thereof optionally further comprise one or more additional genomic edits as described herein, without adversely impacting the differentiation potential of the iPSC and function of the derived effector cells, such as derivative T and NK cells.

Also provided is a master cell bank comprising single cell sorted and expanded clonal engineered iPSCs having at least an exogenously introduced polynucleotide encoding a signaling redirector receptor, and optionally a polynucleotide encoding a cytokine signaling complex as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 2. Chimeric Antigen Receptor (CAR) Expression

Applicable to the genetically engineered iPSC and derivative effector cells thereof may be any CAR design known in the art. CAR is a fusion protein generally including an ectodomain that comprises an antigen recognition domain, a transmembrane domain, and an endodomain. In some embodiments, the ectodomain can further include a signal peptide or leader sequence and/or a spacer. In some embodiments, the endodomain can further comprise a signaling peptide that activates the effector cell expressing the CAR. In some embodiments, the endodomain can further comprise a signaling domain, where the signaling domain originates from a cytoplasmic domain of a signal transducing protein specific to T and/or NK cell activation or functioning. In some embodiments, the antigen recognition domain can specifically bind an antigen. In some embodiments, the antigen recognition domain can specifically bind an antigen associated with a disease or pathogen. In some embodiments, the disease-associated antigen is a tumor antigen, wherein the tumor may be a liquid or a solid tumor. In some embodiments, the CAR is suitable to activate either T or NK lineage cells expressing the CAR. In some embodiments, the CAR is NK cell-specific for comprising NK-specific signaling components. In certain embodiments, the NK cells are derived from iPSCs comprising the CAR. In some embodiments, the CAR is T cell-specific for comprising T cell specific signaling components. In certain embodiments, the T cells are derived from an iPSC comprising the CAR, and the derivative T lineage cells may comprise T helper cells, cytotoxic T cells, memory T cells, regulatory T cells, natural killer T cells, αβ T cells, γδ T cells, or a combination thereof.

In certain embodiments, the antigen recognition region/domain comprises a murine antibody, a human antibody, a humanized antibody, a camel Ig, a shark heavy-chain-only antibody (VNAR), Ig NAR, a chimeric antibody, a recombinant antibody, or an antibody fragment thereof. Non-limiting examples of antibody fragments include Fab, Fab′, F(ab′)2, F(ab′)3, Fv, single chain antigen binding fragment (scFv), (scFv)₂, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody. In some embodiments, the antigen recognition region of a CAR originates from the binding domain of a T cell receptor (TCR) that targets a tumor associated antigen (TAA).

Non-limiting examples of antigens that may be targeted by a CAR include ADGRE2, B7H3, carbonic anhydrase IX (CAIX), CCR1, CCR4, carcinoembryonic antigen (CEA), CD3, CD5, CD7, CD8. CD10. CD20. CD22. CD30, CD33, CD34, CD38, CD41, CD44, CD44V6, CD49f, CD56, CD70, CD74, CD99, CD123, CD133, CD138, CDS, CLECI2A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NYESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigens known in the art, Non-limiting examples of pathogens include viruses, bacteria, fungi, parasites, and protozoa capable of causing diseases.

Thus, in some embodiments, the genetically engineered iPSC and its derivative cells comprise an exogenous polynucleotide encoding a CAR, where the CAR is specific to a target including, but not limited to, B7H3, CD19, BCMA, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1.

In some embodiments, the transmembrane domain of a CAR comprises a full length or at least a portion of the native or modified transmembrane region of CD2, CD38, CD3ε, CD3γ, CD3λ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide.

In some embodiments, the signal transducing peptide of the endodomain (or intracellular domain) comprises a full length or at least a portion of a polypeptide of 2B4 (Natural killer Cell Receptor 2B4), 4-1BB (Tumor necrosis factor receptor superfamily member 9), CD16 (IgG Fc region Receptor III-A), CD2 (T-cell surface antigen CD2), CD28 (T-cell-specific surface glycoprotein CD28). CD28H (Transmembrane and immunoglobulin domain-containing protein 2), CD3ζ (T-cell surface glycoprotein CD3 zeta chain), CD3ζIXX (CD3ζ variant). DAP10 (Hematopoietic cell signal transducer). DAP12 (TYRO protein tyrosine kinase-binding protein), DNAM1 (CD226 antigen), FcERIγ (High affinity immunoglobulin epsilon receptor subunit gamma), IL21R (Interleukin-21 receptor), IL-2Rβ/IL-15RB (Interleukin-2 receptor subunit beta), IL-2Rγ (Cytokine receptor common subunit gamma), IL-7R (Interleukin-7 receptor subunit alpha), KIR2DS2 (Killer cell immunoglobulin-like receptor 2DS2), NKG2D (NKG2-D type II integral membrane protein), NKp30 (Natural cytotoxicity triggering receptor 3), NKp44 (Natural cytotoxicity triggering receptor 2), NKp46 (Natural cytotoxicity triggering receptor 1), CS1(SLAM family member 7), and CD8 (T-cell surface glycoprotein CD8 alpha chain).

In some embodiments, the endodomain of a CAR further comprises a second signaling domain, and optionally a third signaling domain, where each of the first, second, and third signaling domains are different. In particular embodiments, the second and/or the third signaling domain comprises a cytoplasmic domain, or a portion thereof, of 2B4, 4-1BB. CD16, CD2, CD28, CD28H, CD3ζ, DAP10, DAP12, DNAM1, FcERIγ IL21R, IL-2Rβ (IL-15Rβ), IL-2Rγ, IL-7R, KIR2DS2, NKG2D, NKp30, NKp44, NKp46, CD3ζ1XX, CS1, or CD8. In certain embodiments, the endodomain of a CAR further comprises at least one co-stimulatory signaling region. The co-stimulatory signaling region can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In some embodiments, the CAR applicable to the cells provided herein comprises a co-stimulatory domain derived from CD28, and a signaling domain comprising the native or modified ITAM1 of CD3ζ, represented by an amino acid sequence having at least about 85%, about 90%, about 95%, about 96%, about 97%, about 98%, or about 99% identity to SEQ ID NO: 9. In a further embodiment, the CAR comprising a co-stimulatory domain derived from CD28, and a native or modified ITAM1 of CD3ζ also comprises a hinge domain and trans-membrane domain derived from CD28, wherein an scFv may be connected to the trans-membrane domain through the hinge, and the CAR comprises an amino acid sequence of at least about 85%, about 900%, about 95%, about 96%, about 97%, about 98%, or about 99% identity to SEQ ID NO: 10.

SEQ ID NO: 9
RSKRSRLLHSDYMNMTPRRPGPTRKHYQPYAPPRDFAAYRSRVKESRSADAPAYQQGQNQ
LYNELNLGRREEYDVLDKRRGRDPEMGGKPRRKNPQEGLFNELQKDKMAEAFSEIGMKGE
RRRGKGHDGLFQGLSTATKDTFDALHMQALPPR
(153 a.a. CD28 co-stim + CD3ζITAM)
SEQ ID NO: 10
IEVMYPPPYLDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLFNELQKDKMARAFSE
IGMKGERRRGKGHDGLFQGLSTATKDTFDALHMQALPPR
(219 a.a. CD28 hinge + CD28 TM + CD28 co-stim + CD3ζITAM)

In various embodiments, the CAR applicable to the cells provided herein comprises a transmembrane domain derived from NKG2D, a co-stimulatory domain derived from 2B4, and a signaling domain comprising the native or modified CD3ζ, represented by an amino acid sequence of at least about 85%, about 90%, about 95%, about 96%, about 97%, about 98%, or about 99% identity to SEQ ID NO: 11. The CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 12.

SEQ ID NO: 11
SNLFVASWIAVMIIFRIGMAVAIFCCFFFPSWRRKRKEKQSETSPKEFLTIYEDVKDLKT
RRNHEQEQTFPGGGSTIYSMIQSQSSAPTSQEPAYTLYSLIQPSRKSGSRKRNHSPSFNS
TIYEVIGKSQPKAQNPARLSRKELENFDVYSRVKFSRSADAPAYKQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPR
(263 a.a NKG2D TM + 2B4 + CD32)
SEQ ID NO: 12
TTTPAPRPPTPAPTIASQPLSLRPEACRPAAGGAVHTRGLDFACDSNLFVASWIAVMIIF
RIGMAVAIFCCFFFPSWRRKRKEKQSETSPKEFLTIYEDVKDLKTRRNHEQEQTFPGGGS
TIYSMIQSQSSAPTSQEPAYTLYSLIQPSRKSGSRKRNHSPSFNSTIYEVIGKSQPKAQN
PARLSRKELENFDVYSRVKFSRSADAPAYKQGQNQLYNELNLGRREEYDVLDKRRGRDPE
MGGKPRRKNPQEGLYNELQKDKMAEAYSEIGMKGERRRGKGHDGLYQGLSTATKDTYDAL
HMQALPPR
(308 a.a CD8 hinge + NKG2D TM + 2B4 + CD3ζ)

Non-limiting CAR strategies further include heterodimeric, conditionally activated CAR through dimerization of a pair of intracellular domains (see for example, U.S. Pat. No. 9,587,020); split CAR, where homologous recombination of antigen binding, hinge, and endodomains to generate a CAR (see for example, U.S. Pub. No. 2017/0183407); multi-chain CAR that allows non-covalent link between two transmembrane domains connected to an antigen binding domain and a signaling domain, respectively (see for example, U.S. Pub. No. 2014/0134142); CARs having bispecific antigen binding domain (see for example, U.S. Pat. No. 9,447,194), or having a pair of antigen binding domains recognizing same or different antigens or epitopes (see for example, U.S. Pat. No. 8,409,577), or a tandem CAR (see for example, Hegde et al., J Clin Invest. 2016; 126(8):3036-3052); inducible CAR (see for example, U.S. Pub. Nos. 2016/0046700, 2016/0058857, and 2017/0166877); switchable CAR (see for example, U.S. Pub. No. 2014/0219975); and any other designs known in the art.

In a further embodiment, the iPSC and its derivative effector cells comprising a signaling redirector receptor and optionally a CAR have the CAR inserted in a TCR constant region, leading to endogenous TCR knockout (TCR^{KO}), and optionally placing CAR expression under the control of the endogenous TCR promoter. In some other embodiments, the CAR inserted in the TCR constant region is specific to a tumor antigen comprising at least one of MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1. Additional CAR insertion sites include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT.

As such, aspects of the present invention provide genomically engineered iPSCs and derivative cells obtained from differentiating the genomically engineered iPSCs, wherein the iPSCs and the derivative cells comprise an exogenous polynucleotide encoding a signaling redirector receptor (“SRR” in Table 1) and optionally one or more of an exogenous cytokine signaling complex as described herein (“IL” in Table 1), a CAR and/or one or more additional modified modalities, as provided in Table 1 without adversely impacting the differentiation potential of the iPSC and function of the derived effector cells, such as derivative T and NK cells. In iPSCs and derivative cells therefrom comprising both an exogenous cytokine signaling complex (“IL” in Table 1) and a CAR, the IL and CAR may be expressed in separate constructs, or may be co-expressed in a bi-cistronic construct comprising both IL and CAR. Also provided is a master cell bank comprising single cell sorted and expanded clonal engineered iPSCs having at least an exogenously introduced polynucleotide encoding a signaling redirector receptor, and a polynucleotide encoding a CAR, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 3. CD16 Knock-In

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” or “high affinity non-cleavable CD16,” as used herein, refers to various CD16 variants. The wildtype CD16 has low affinity and is subject to ectodomain shedding, a proteolytic cleavage process that regulates cell surface density of various cell surface molecules on leukocytes upon NK cell activation. F176V (also called F158V in some publications) is an exemplary CD16 polymorphic variant having high affinity; whereas an S197P variant is an example of a genetically engineered non-cleavable version of CD16. An engineered CD16 variant comprising both F176V and S197P has high affinity and is non-cleavable, which was described in greater detail in International Pub. No. WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

Unlike the endogenous CD16 expressed by primary NK cells which gets cleaved from the cellular surface following NK cell activation, the various non-cleavable versions of CD16 in derivative NK cells avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, in some embodiments, the hnCD16 may comprise F176V and S197P, or may comprise a full or partial length ectodomain originated from CD64, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed below.

Unlike primary NK cells, mature T cells from a primary source (i.e., natural/native sources such as peripheral blood, umbilical cord blood, or other donor tissues) do not express CD16. It was unexpected that an iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR (chimerical antigen receptor). When said derivative T lineage cell comprises acquired ADCC through exogenous CD16, including functional variants and CD16-based CFcR expression, and when an antibody targets a different tumor antigen from the one targeted by the CAR the antibody can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, a hnCD16 comprises a sequence having an identity of at least 50%, 55%, 60%, 65%, 70%, 75%, 80%, 85%, 90%, 95%, 99%, 100%, or any percentage in-between, when compared to any of the exemplary sequences, SEQ ID NOs: 13, 14 and 15, each comprising at least a portion of CD64 ectodomain.

SEQ ID NO: 13
MWFLTTLLLWVPVDGQVDTTKAVITLQPPWVSVFQEETVTLHCEVLHLPGSSSTQWFLNGTATQTSTPSY
RITSASVNDSGEYRCQRGLSGRSDPIQLEIHRGWLLLQVSSRVFTEGEPLALRCHAWKDKLVYNVLYYRN
GKAFKFFHWNSNLTILKTNISHNGTYHCSGMGKHRYTSAGISVTVKELFPAPVLNASVTSPLLEGNLVTL
SCETKLLLQRPGLQLYFSFYMGSKTLRGRNTSSEYQILTARREDSGLYWCEAATEDGNVLKRSPELELQV
LGLQLPTPVWFHYQVSFCLVMVLLFAVDIGLYFSVKTNIRSSTRDWKDHKFKWRKDPQDK
(340 a.a. CD64 domain-based construction; CD16TM; CD16ICD)
SEQ ID NO: 14
MWFLTTLLLWVPVDGQVDTTKAVITLQPPWVSVFQEETVTLHCEVLHLPGSSSTQWFLNGTATQTSTPSY
RITSASVNDSGEYRCQRGLSGRSDPIQLEIHRGWLLLQVSSRVFTEGEPLALRCHAWKDKLVYNVLYYRN
GKAFKFFHWNSNLTILKTNISHNGTYHCSGMGKHRYTSAGISVTVKELFPAPVLNASVTSPLLEGNLVTL
SCETKLLLQRPGLQLYFSFYMGSKTLRGRNTSSEYQILTARREDSGLYWCEAATEDGNVLKRSPELELQV
LGLFFPPGYQVSFCLVMVLLFAVDIGLYESVKTNIRSSTRDWKDHKEKWRKDPQDK
(336 a.a. CD64 exon-based construction; CD16TM; CD16ICD)
SEQ ID NO: 15
MWFLTTLLLWVPVDGQVDTTKAVITLQPPWVSVFQEETVTLHCEVLHLPGSSSTQWFLNG
TATQTSTPSYRITSASVNDSGEYRCQRGLSGRSDPIQLEIHRGWLLLQVSSRVFTEGEPL
ALRCHAWKDKLVYNVLYYRNGKAFKEFHWNSNLTILKTNISHNGTYHCSGMGKHRYTSAG
ISVTVKELFPAPVLNASVTSPLLEGNLVTLSCETKLLLQRPGLQLYFSFYMGSKTLRGRN
TSSEYQILTARREDSGLYWCEAATEDGNVLKRSPELELQVLGFFPPGYQVSFCLVMVLLF
AVDTGLYFSVKTNIRSSTRDWKDHKFKWRKDPQDK
(335 a.a. CD64 exon-based construction; CD16TM; CD16ICD)

Accordingly, provided herein are iPSCs genetically engineered to comprise, among other editing as contemplated and described herein, an exogenous CD16 that is a high-affinity non-cleavable CD16 receptor (hnCD16), wherein the genetically engineered iPSCs are capable of differentiating into effector cells comprising the hnCD16 introduced to the iPSCs. In some embodiments, the derived effector cells comprising SRR and exogenous CD16, and optionally one or more of IL and CAR are NK cells. In some embodiments, the derived effector cells comprising SRR and exogenous CD16, and optionally one or more of IL and CAR are T cells.

The exogenous hnCD16 expressed in iPSC or derivative cells thereof has high affinity in binding to not only ADCC antibodies or fragments thereof, but also to bi-, tri-, or multi-specific engagers or binders that recognize the CD16 or CD64 extracellular binding domains of the hnCD16. The bi-, tri-, or multi-specific engagers or binders are further described below in this application. As such, the present application provides a derivative effector cell or a cell population thereof, preloaded with one or more pre-selected ADCC antibodies through high-affinity binding with the extracellular domain of the hnCD16 expressed on the derivative effector cell, in an amount sufficient for therapeutic use in a treatment of a condition, a disease, or an infection as further detailed below, wherein said hnCD16 comprises an extracellular binding domain of CD64, or of CD16 having F176V and S197P. Thus, in some embodiments, the derived NK cells are preloaded with an antibody. In some embodiments, the derived NK cells are used in a combination therapy with an antibody. In some embodiments, the antibody in the combination therapy or preloaded with the derived NK cells specifically targets CD38. In some embodiments, the antibody in the combination therapy or preloaded with the derived NK cells specifically targets an antigen different from CD38. In some embodiments, the anti-CD38 antibody is daratumumab.

In some other embodiments, the exogenous CD16 expressed in iPSC or derivative cells thereof comprises a CD16-, or variants thereof, based CFcR. A chimeric Fc receptor (CFcR) is produced to comprise a non-native transmembrane domain, a non-native stimulatory domain and/or a non-native signaling domain by modifying or replacing the native CD16 transmembrane- and/or the intracellular-domain. The term “non-native” used herein means that the transmembrane, stimulatory or signaling domain are derived from a different receptor other than the receptor which provides the extracellular domain. In the illustration here, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide. In some embodiments, the exogenous CD16-based CFcR comprises a non-native stimulator/inhibitors domain derived from CD27. CD28, 4-1BB, OX40, ICOS, PD-1, LAG-3, 2B4, BTLA, DAP10. DAP12, CTLA-4, or NKG2D polypeptide. In some embodiments, the exogenous CD16-based CFcR comprises anon-native signaling domain derived from CD3ζ, 2B4, DAP10, DAP12, DNAM1, CD137 (4-1BB), IL21, IL7, IL12, IL15. NKp30, NKp44. NKp46. NKG2C, or NKG22D polypeptide. In some embodiments of the CD16-based CFcR, the provided chimeric Fc receptor comprises a transmembrane domain and a signaling domain both derived from one of IL7. IL12, IL15. NKp30, NKp44, NKp46. NKG2C, or NKG2D polypeptide. One particular exemplary embodiment of the CD16-based chimeric Fc receptor comprises a transmembrane domain of NKG2D, a stimulatory domain of 2B4, and a signaling domain of CD3ζ; wherein the extracellular domain of the CFcR is derived from a full length or partial sequence of the extracellular domain of CD64 or CD16, and wherein the extracellular domain of CD16 comprises F176V and S197P. Another exemplary embodiment of the CD16-based chimeric Fc receptor comprises a transmembrane domain and a signaling domain of CD3ζ; wherein the extracellular domain of the CFcR is derived from a full length or partial sequence of the extracellular domain of CD64 or CD16, and wherein the extracellular domain of CD16 comprises F176V and S197P.

The various embodiments of CD16-based chimeric Fc receptor as described above are capable of binding, with high affinity, to the Fc region of an antibody or fragment thereof; or to a bi-, tri-, or multi-specific engager or binder. Upon binding, the stimulatory and/or signaling domains of the chimeric receptor enable the activation and cytokine secretion of the effector cells, and the killing of the tumor cells targeted by the antibody, or the bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into a close proximity, which also contributes to the enhanced killing of the tumor cells. Exemplary tumor antigens for bi-, tri-, multi-specific engagers or binders include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EPCAM, and CD16 (or CD64)-IL15-CD33.

Accordingly, in some embodiments, the present invention provides iPSCs and derivative cells therefrom comprising a signaling redirector receptor and an exogenous CD16 or a variant thereof as described herein (“CD16^{exo}” in Table 1), and optionally one or more of an exogenous cytokine signaling complex as described herein and a CAR, wherein the cells, such as derivative T and NK cells, have improved resistance to cytokine immunosuppression in an adoptive cell therapy for solid tumors. In some embodiments, the iPSC and derivative cells thereof further comprise one or more additional genomic edits as described herein, without adversely impacting the differentiation potential of the iPSC and function of the derived effector cells, such as derivative T and NK cells.

Also provided is a master cell bank comprising single cell sorted and expanded clonal engineered iPSCs having at least an exogenously introduced polynucleotide encoding a signaling redirector receptor and a polynucleotide encoding an exogenous CD16 or a variant thereof, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 4. CD38 Knock-Out

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include antibody-dependent cell-mediated phagocytosis (ADCP) and/or complement-dependent cytotoxicity (CDC).

Other than being highly expressed on malignant cells, CD38 is also expressed on plasma cells as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme. CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the calcium dependent process of cell adhesion. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells however have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38-specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114).

Without being limited by theories, the present application provides a strategy to leverage the full potential of CD38-targeted cancer treatment by knocking out CD38 in the effector cell, thereby overcoming CD38-specific antibody and/or CD38 antigen binding domain-induced effector cell depletion or reduction through fratricide. In addition, since CD38 is upregulated on activated lymphocytes such as T or B cells, by suppressing activation of these recipient lymphocytes using a CD38-specific antibody, such as daratumumab, in the recipient of allogeneic effector cells, host allorejection against these effector cells would be reduced and/or prevented, thereby increasing effector cell survival and persistency. As such, a CD38-specific antibody, a secreted CD38-specific engager or a CD38-CAR (chimeric antigen receptor) against activation of recipient T, Treg, NK, and/or B cells can be used as a replacement for lymphodepletion using chemotherapy such as Cy/Flu (cyclophosphamide/fludarabine) prior to adoptive cell transferring. In addition, when targeting CD38⁺ T and pbNK cells using CD38-effector cells in the presence of anti-CD38 antibodies or CD38 inhibitors, the depletion of CD38⁺ alloreactive cells increases the NAD⁺ (nicotinamide adenine dinucleotide, a substrate of CD38) availability and decreases NAD⁺ consumption related cell death, which, among other advantages, boosts effector cell responses in an immunosuppressive tumor microenvironment and supports cell rejuvenation in aging, degenerative or inflammatory diseases.

Thus, the strategies as provided herein also include generating an iPSC line comprising a signaling redirector receptor and CD38 knock-out (“CD38^{-/-}” in Table 1), and obtaining derivative effector cells comprising IL and CD38 null (CD38^{-/-}) and optionally a CAR, through directed differentiation of the engineered iPSC line. In one embodiment, the CD38 knock-out in an iPSC line is a bi-allelic knock-out.

As disclosed herein, in some embodiments, the provided iPSC line comprising a signaling redirector receptor and CD38 knockout, and optionally one or more of an exogenous cytokine signaling complex as described herein, a CAR, an exogenous CD16 or variant thereof, is capable of directed differentiation to produce functional derivative hematopoietic cells including, but not limited to, mesodermal cells with definitive hemogenic endothelium (HE) potential, definitive HE. CD34 hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages. In some embodiments, when an anti-CD38 antibody is used to induce ADCC or an anti-CD38 CAR is used for targeted cell killing, the iPSC and/or derivative effector cells thereof comprising SRR, and CD38^{-/-} are not eliminated by the anti-CD38 antibody or the anti-CD38 CAR, thereby increasing the iPSC and its effector cell persistence and/or survival in the presence of, and/or after exposure to, such therapeutic agents. In some embodiments, the effector cell has increased persistence and/or survival in vivo in the presence of, and/or after exposure to, such therapeutic agents.

In some embodiments, the derived effector cells comprising a signaling redirector receptor and CD38^{-/-}, and optionally one or more of an exogenous cytokine signaling complex as described herein, a CAR, and an exogenous CD16 or a variant thereof, are NK cells derived from iPSCs. In some embodiments, the effector cells comprising a signaling redirector receptor and CD38^{-/-}, and optionally one or more of an exogenous cytokine signaling complex as described herein, a CAR, and an exogenous CD16 or a variant thereof, are T cells derived from iPSCs. In some embodiments, the iPSC and/or derivative cells thereof comprising a signaling redirector receptor and CD38^{-/-}, and optionally one or more of an exogenous cytokine signaling complex as described herein, a CAR, and an exogenous CD16 or a variant thereof, further comprise one or more additional genomic edits as provided in Table 1, without adversely impacting the differentiation potential of the iPSC and function of the derived effector cells, such as derivative T and NK cells.

Also provided is a master cell bank comprising single cell sorted and expanded clonal engineered iPSCs having a polynucleotide encoding a signaling redirector receptor and a CD38 knockout, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 5. HLA-I- and HA-II-Deficiency

Multiple HLA class I and class 11 proteins must be matched for histocompatibility in allogeneic recipients to avoid allogeneic rejection problems. Provided herein is an iPSC cell line with eliminated or substantially reduced expression of one or both HLA class I (“HLA-I”) and HLA class II (“HLA-II”) proteins. HLA class I deficiency can be achieved by functional deletion of any region of the HLA class I locus (chromosome 6p21), or deletion or reducing the expression level of HLA class-I associated genes including, not being limited to, beta-2 microglobulin (B2M) gene, TAP1 gene, TAP2 gene and Tapasin. For example, the B2M gene encodes a common subunit essential for cell surface expression of all HLA class I heterodimers. B2M negative cells are HLA-1 deficient.

HLA class II deficiency can be achieved by functional deletion or reduction of HLA-II associated genes including, not being limited to, RFXANK, CIITA, RFX5 and RFXAP. CIITA is a transcriptional coactivator, functioning through activation of the transcription factor RFX5 required for class II protein expression. CIITA negative cells are HLA-II deficient. However, lacking HLA class I expression increases susceptibility to lysis by NK cells. As such, this application provides an iPSC and derivative cells therefrom comprising HLA-1 and/or HLA-II deficiency, for example by lacking B2M and/or CIITA expression, wherein the obtained derivative effector cells enable allogeneic cell therapies by eliminating the need for MHC (major histocompatibility complex) matching, and avoiding recognition and killing by host (allogeneic) T cells.

Furthermore, a lack of HLA class I expression leads to lysis by host NK cells. Therefore, in addition to the above-discussed approach of CD38 conditioning to remove activated CD38-expressing host NK cells, to overcome this “missing self” response, HLA-E. HLA-G, or other non-classical HLA-I proteins may be optionally knocked in to avoid NK cell recognition and killing of the HLA-I deficient effector cells derived from an engineered iPSC. In one embodiment, the provided HLA-1 deficient iPSC and its derivative cells further comprise HLA-G knock-in. Alternatively, or in addition thereto, the provided HLA-I deficient iPSC and its derivative cells further comprise one or both of CD58 knockout and CD54 knockout. CD58 (or LFA-3) and CD54 (or ICAM-1) are adhesion proteins initiating signal-dependent cell interactions, and facilitating cell, including immune cell, migration. It was previously shown that CD58 and/or CD54 disruption effectively reduces the susceptibility of HLA-I deficient iPSC-derived effector cells to allogeneic NK cell killing. While it was shown that CD58 knockout has a higher efficiency in reducing allogeneic NK cell activation than CD54 knockout, double knockout of both CD58 and CD54 was shown to provide the most enhanced reduction of NK cell activation. In some observations, the CD58 and CD54 double knockout is even more effective than HLA-G overexpression for HLA-1 deficient cells in overcoming “missing-self” effect.

Accordingly, in some embodiments, the present invention provides a strategy to enhance effector cell persistency and/or survival through reducing or preventing allorejection by generating HLA-1 and/or HLA-II deficiency, optionally with additional HLA-I protein modifications, without adversely impacting the differentiation potential of the iPSC and function of the derived effector cells, such as derivative T and NK cells.

In some embodiments, the effector cells have increased persistence and/or survival in vivo in the presence of, and/or after exposure to, therapeutic agents. Thus, in some embodiments, the iPSC and derivative cells thereof comprising a signaling redirector receptor and a HLA modification (“HLA” in Table 1: HLA-I and/or II deficiency, with or without HLA-E or HLA-G knock in, or with knockout of one or both of CD58 and CD54), and optionally one or more of an exogenous cytokine signaling complex as described herein, a CAR, an exogenous CD16 or a variant thereof, and CD38^{-/-}. In some embodiments, the iPSC and derivative cells thereof comprising a signaling redirector receptor, and optionally one or more of an exogenous cytokine signaling complex as described herein, a CAR, an exogenous CD16 or a variant thereof, and CD38^{-/-}, are HLA-I deficient and HLA-II deficient (e.g., B2M^{-/-} and CIITA negative or CIITA^{-/-}). In some embodiments, the iPSC and derivative cells thereof comprising a signaling redirector receptor and optionally one or more of an exogenous cytokine signaling complex as described herein, a CAR, an exogenous CD16 or a variant thereof, and CD38^{-/-}, are HLA-I and/or HLA-II deficient and further comprise an exogenous polynucleotide encoding HLA-G or HLA-E. In some embodiments, the iPSC and derivative cells thereof comprising a signaling redirector receptor and optionally one or more of an exogenous cytokine signaling complex as described herein, a CAR, an exogenous CD16 or a variant thereof, and CD38^{-/-}, are HLA-I and/or HLA-II deficient and are CD58 negative. In some embodiments, the iPSC and derivative cells thereof comprising a signaling redirector receptor and optionally one or more of an exogenous cytokine signaling complex as described herein, a CAR, an exogenous CD16 or a variant thereof, and CD38^{-/-}, are HLA-I and/or HLA-II deficient and are CD54 negative. In some embodiments, the iPSC and derivative cells thereof comprising a signaling redirector receptor and optionally one or more of an exogenous cytokine signaling complex as described herein, a CAR, an exogenous CD16 or a variant thereof, and CD38^{-/-}, are HLA-I and/or HLA-II deficient and are both CD58 negative and CD54 negative.

In some embodiments, the effector cells comprising a signaling redirector receptor and a HLA modification, and optionally one or more of an exogenous cytokine signaling complex, a CAR, an exogenous CD16 or a variant thereof. CD38^{-/-}, are NK cells derived from iPSCs. In some embodiments, the effector cells comprising a signaling redirector receptor and a HLA modification, and optionally one or more of an exogenous cytokine signaling complex, a CAR, an exogenous CD16 or a variant thereof, and CD38^{-/-} are T cells derived from iPSCs.

Also provided is a master cell bank comprising single cell sorted and expanded clonal engineered iPSCs having a polynucleotide encoding a signaling redirector receptor and a HLA modification as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 6. Engagers

Engagers are fusion proteins consisting of two or more single-chain variable fragments (scFvs), or other functional variants, of different antibodies or fragments thereof, with at least one scFv that binds to an effector cell surface molecule or surface triggering receptor, and at least another to a target cell via a target cell specific surface molecule. Examples of engagers include, but are not limited to, bi-specific T cell engagers (BiTEs), bi-specific killer cell engagers (BiKEs), tri-specific killer cell engagers (TriKEs), multi-specific killer cell engagers, or universal engagers compatible with multiple immune cell types. Engagers can be bi-specific or multi-specific. Such bi-specific or multi-specific engagers are capable of directing an effector cell (e.g., a T cell, a NK cell, an NKT cell, a B cell, a macrophage, and/or a neutrophil) to a tumor cell and activating the immune effector cell, and have shown great potential to maximize the benefits of CAR-T cell therapy.

In some embodiments, the engager is used in combination with a population of the effector cells described herein by concurrent or consecutive administration, wherein the effector cells comprise a surface molecule, or surface triggering receptor, that is recognized by the engager. In some other embodiments, the engager (“En⁺” in Table 1) is a bi-specific antibody expressed by a derivative effector cell through genetically engineering an iPSC as described herein using an exogenous polynucleotide sequence encoding the engager, or a fragment or variant thereof, and directed differentiation of the engineered iPSC. Exemplary effector cell surface molecules, or surface triggering receptors, that can be used for bi- or multi-specific engager recognition, or coupling thereof, include, but are not limited to, CD3, CD28, CD5, CD16, NKG2D, CD64, CD32, CD89, NKG2C, and a chimeric Fc receptor as disclosed herein. In some embodiments, the exogenous CD16 expressed on the surface of the derivative effector cells for engager recognition is a hnCD16, comprising a CD16 (containing F176V and optionally S197P) or a CD64 extracellular domain, and native or non-native transmembrane, stimulatory and/or signaling domains as described herein. In some embodiments, the CD16 expressed on the surface of effector cells for engager recognition is a CD16-based chimeric Fc receptor (CFcR). In some embodiments, the CD16-based CFcR comprises a transmembrane domain of NKG2D, a stimulatory domain of 2B4, and a signaling domain of CD3′; wherein the extracellular domain of the CD16 is derived from a full length or partial sequence of the extracellular domain of CD64 or CD16; and wherein the extracellular domain of CD16 comprises F176V and optionally S197P.

In some embodiments, the target cell for an engager is a tumor cell. The exemplary tumor cell surface molecules for bi- or multi-specific engager recognition include, but are not limited to, B7H3, BCMA, CD10. CD19. CD20. CD22. CD24. CD30. CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and RORL. In one embodiment, the bi-specific engager is a bi-specific antibody specific to CD3 and CD19 (CD3-CD19). In another embodiment, the bi-specific antibody is CD16-CD30 or CD64-CD30. In another embodiment, the bi-specific antibody is CD16-BCMA or CD64-BCMA. In still another embodiment, the bi-specific antibody is CD3-CD33.

In yet another embodiment, the bi-specific antibody further comprises a linker between the effector cell and tumor cell antigen binding domains. For example, a modified IL15 may be used as a linker for effector NK cells to facilitate cell expansion (called TriKE, or Tri-specific Killer Engager, in some publications). In one embodiment, the TriKE is CD16-IL15-EPCAM or CD64-IL15-EPCAM. In another embodiment, the TriKE is CD16-IL15-CD33 or CD64-IL15-CD33. In yet another embodiment, the TriKE is NKG2C-IL15-CD33. The IL15 in the TriKE may also originate from other cytokines including, but not limited to, IL2, IL4, IL6, IL7, IL9, IL10, IL11, IL12, IL18, and IL21.

In some embodiments, the surface triggering receptor for a bi- or multi-specific engager could be endogenous to the effector cells, sometimes depending on the cell types. In some other embodiments, one or more exogenous surface triggering receptors could be introduced to the effector cells using the methods and compositions provided herein, e.g., through additional engineering of an iPSC comprising a genotype listed in Table 1, then directing the differentiation of the iPSC to T, NK or any other effector cells comprising the same genotype and the surface triggering receptor as the source iPSC.

As such, aspects of the present invention provide genomically engineered iPSCs and derivative cells obtained from differentiating the genomically engineered iPSCs, wherein the iPSCs and the derivative cells comprise an exogenous polynucleotide encoding a signaling redirector receptor and an engager, and optionally one or more of an exogenous cytokine signaling complex, a CAR, an exogenous CD16 or a variant thereof, CD38 knockout, HLA modification, and/or one or more additional modified modalities, as provided in Table 1, without adversely impacting the differentiation potential of the iPSC and function of the derived effector cells, such as derivative T and NK cells.

Also provided is a master cell bank comprising single cell sorted and expanded clonal engineered iPSCs having at least an exogenously introduced polynucleotide encoding a signaling redirector receptor and a polynucleotide encoding an engager, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 7. Antibodies for Immunotherapy

In some embodiments, in addition to the genomically engineered effector cells as provided herein, additional therapeutic agents comprising an antibody, or an antibody fragment that targets an antigen associated with a condition, a disease, or an indication may be used with these effector cells in a combinational therapy. In some embodiments, the antibody is used in combination with a population of the effector cells described herein by concurrent or consecutive administration to a subject. In other embodiments, such antibody or a fragment thereof (“Ab⁺” in Table 1) may be expressed by the effector cells by genetically engineering an iPSC using an exogenous polynucleotide sequence encoding the antibody or fragment thereof, and directing differentiation of the engineered iPSC. In some embodiments, the effector cell expresses an exogenous CD16 variant, wherein the cytotoxicity of the effector cell is enhanced by the antibody via ADCC.

In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor- or viral-specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 (rituximab, veltuzumab, ofatumumab, ublituximab, ocaratuzumab, obinutuzumab), anti-HER2 (trastuzumab, pertuzumab), anti-CD52 (alemtuzumab), anti-EGFR (cetuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab); and their humanized or Fc modified variants or fragments, or their functional equivalents and biosimilars.

In some embodiments, the iPSC-derived effector cells comprise hematopoietic lineage cells comprising a genotype listed in Table 1. In some embodiments, the iPSC-derived effector cells comprise NK cells comprising a genotype listed in Table 1. In some embodiments, the iPSC-derived effector cells comprise T cells comprising a genotype listed in Table 1. In some embodiments of a combination useful for treating liquid or solid tumors, the combination comprises iPSC-derived NK or T cells comprising at least IL and one or more modalities provided in Table 1. In one embodiment, the combination comprises iPSC-derived NK or T cells comprising a signaling redirecting receptor (“SRR”), and optionally one or more of an additional cytokine signaling complex (“IL”), a CAR, CD38 knockout (“CD38^{-/-}”), an exogenous CD16 or a variant thereof (“CD16^{exo}”), and HLA modification (“HLA”); and optionally one of the anti-CD38 antibodies, daratumumab, isatuximab, and MOR202 (“Ab⁺”). In one embodiment, the combination comprises iPSC-derived NK cells comprising a signaling redirecting receptor and daratumumab, and optionally one or more of an cytokine signaling complex, a CAR, CD38 knockout, an exogenous CD16 or a variant thereof, and HLA deficiency. In some further embodiments, the iPSC-derived NK cells comprised in the combination with one of the anti-CD38 antibodies comprise a TGFβ-SRR, and optionally one or more of an IL15 signaling complex, a CAR. CD38 knockout, an exogenous CD16 or a variant thereof, and HLA modification: wherein the IL15 signaling complex is co- or separately expressed with the CAR; and IL15 cytokine signaling complex is in any one of the forms described herein. In some particular embodiments, IL15 cytokine signaling complex is co- or separately expressed with the CAR. In some further embodiments, the iPSC-derived NK cells comprised in the combination with one of the anti-CD38 antibodies comprise a signaling redirecting receptor and optionally one or more of an IL7 signaling complex, a CAR, CD38 knockout, an exogenous CD16 or a variant thereof, HLA modification; wherein the IL7 signaling complex is co- or separately expressed with the CAR; and IL7 signaling complex is in any one of the forms described herein. In some particular embodiments, IL7 signaling complex is co- or separately expressed with the CAR.

As such, aspects of the present invention provide genomically engineered iPSCs and derivative cells obtained from differentiating the genomically engineered iPSCs, wherein the iPSCs and the derivative cells comprise an exogenous polynucleotide encoding a signaling redirector receptor and an antibody or a functional fragment thereof, and optionally one or more of an exogenous cytokine signaling complex, a CAR, an exogenous CD16 or a variant thereof, CD38 knockout, HLA modification, and/or one or more additional modified modalities, as provided in Table 1, without adversely impacting the differentiation potential of the iPSC and function of the derived effector cells, such as derivative T and NK cells.

Also provided is a master cell bank comprising single cell sorted and expanded clonal engineered iPSCs having at least an exogenously introduced polynucleotide encoding a signaling redirector receptor and a polynucleotide encoding an antibody or functional fragment thereof, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 8. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or decreasing activity of checkpoint molecules, thereby blocking inhibitory checkpoints, and restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern. One aspect of the present application provides a therapeutic approach to overcome CI resistance by including genomically-engineered functional derivative cells as provided herein in a combination therapy with CI. In some embodiments, the checkpoint inhibitor is used in combination with a population of the effector cells described herein by concurrent or consecutive administration thereof to a subject. In some other embodiments, the checkpoint inhibitor is expressed by the effector cells by genetically engineering an iPSC using an exogenous polynucleotide sequence encoding the checkpoint inhibitor, or a fragment or variant thereof (“CI⁺” in Table 1), and directing differentiation of the engineered iPSC. Some embodiments of the combination therapy with the effector cells described herein comprise at least one checkpoint inhibitor to target at least one checkpoint molecule; wherein the derivative cells have a genotype listed in Table 1.

In some embodiments, the exogenous polynucleotide sequence encoding the checkpoint inhibitor, or a fragment thereof, is co-expressed with a CAR, either in separate constructs or in a bi-cistronic construct. In some further embodiments, the sequence encoding the checkpoint inhibitor, or the fragment thereof, can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR are in a single open reading frame (ORF). When the checkpoint inhibitor is delivered, expressed, and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In one embodiment of the combination therapy, the derivative effector cells comprising a genotype listed in Table 1 are NK lineage cells. In another embodiment of the combination therapy, the derivative effector cells comprising a genotype listed in Table 1 are T lineage cells.

Suitable checkpoint inhibitors for combination therapy with the derivative effector cells as provided herein include, but are not limited to, antagonists of PD-1 (Pdcdl, CD279), PDL-1 (CD274), TIM-3 (Havcr2), TIGIT (WUCAM and Vstm3), LAG-3 (CD223), CTLA-4 (CD152), 2B4 (CD244), 4-1BB (CD137), 4-1BBL (CD137L), AMR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

In some embodiments, the checkpoint inhibitor (“CI⁺” in Table 1) is co-expressed with CAR and inhibits at least one of the following checkpoint molecules: PD-1, PDL-1. TIM-3, TIGIT, LAG-3, CTLA-4, 2B4, 4-1BB, 4-1BBL, AzAR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR. In some embodiments, the checkpoint inhibitor co-expressed with CAR in a derivative cell having a genotype listed in Table 1 is selected from the group comprising atezolizumab, avelumab, durvalumab, tremelimunmab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the derivative effector cells provided herein and at least one antibody inhibiting a checkpoint molecule, the antibody is not produced by, or in, the derivative cells and is additionally administered before, with, or after the administering of the derivative cells as provided herein. In some embodiments, the administering of one, two, three or more checkpoint inhibitors in a combination therapy with the provided derivative NK lineage cells or T lineage cells are simultaneous or sequential. In one embodiment of the combinational treatment, the checkpoint inhibitor included in the treatment is one or more of atezolizumab, avelumab, durvalumab, tremelimumab, ipilimumab, IPH4102, IPH43, IPH33, lirilumab, monalizumab, nivolumab, pembrolizumab, and their humanized or Fc modified variants, fragments and their functional equivalents or biosimilars. In some embodiments of the combination treatment, the checkpoint inhibitor included in the treatment is atezolizumab, or its humanized or Fc modified variant, fragment and its functional equivalent or biosimilar. In some embodiments of the combination treatment, the checkpoint inhibitor included in the treatment is nivolumab, or its humanized or Fc modified variant, fragment or its functional equivalent or biosimilar. In some embodiments of the combination treatment, the checkpoint inhibitor included in the treatment is pembrolizumab, or its humanized or Fc modified variant, fragment or its functional equivalent or biosimilar.

As such, aspects of the present invention provide genomically engineered iPSCs and derivative cells obtained from differentiating the genomically engineered iPSCs, wherein the iPSCs and the derivative cells comprise an exogenous polynucleotide encoding a signaling redirector receptor and a checkpoint inhibitor (CI) or a functional fragment thereof, and optionally one or more of an exogenous cytokine signaling complex, a CAR, an exogenous CD16 or a variant thereof, CD38 knockout. HLA modification, and/or one or more additional modified modalities, as provided in Table 1, without adversely impacting the differentiation potential of the iPSC and function of the derived effector cells, such as derivative T and NK cells.

Also provided is a master cell bank comprising single cell sorted and expanded clonal engineered iPSCs having at least an exogenously introduced polynucleotide encoding a signaling redirector receptor and a polynucleotide encoding encoding a checkpoint inhibitor, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 9. Genetically Engineered iPSC Line and Derivative Cells Provided Herein

In light of the above, the present application provides an iPSC, a clonal iPS cell, an iPS cell line cell, or a derivative cell therefrom, wherein each cell comprises a signaling redirector receptor (“SSR” in Table 1) as described herein. In some embodiments, the signaling redirector receptor comprises a partial or full peptide of an extracellular domain (ECD) of a signaling receptor, and an intracellular domain (ICD) comprising a partial or full peptide of a cytokine receptor, wherein the signaling receptor and the cytokine receptor are different molecules. In various embodiments, the signaling redirector receptor comprises (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R, IL12R, IL18R, IL21R, or any combination thereof, and wherein the derivative cells are functional effector cells obtained from differentiation of an engineered iPSC comprising SSR, and optionally one or more of CD38^{-/-}. HLA deficiency, and exogenous polynucleotides encoding one or more of a cytokine signaling complex, a CAR, an exogenous CD16 or a variant thereof, an antibody, a checkpoint inhibitor, an engager, and any other modality, as shown in Table 1. In some embodiments, the derivative cells are hematopoietic lineage cells including, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T lineage cells, NKT lineage cells, NK lineage cells, B lineage cells, neutrophils, dendritic cells, and macrophages. In some embodiments, the functional derivative hematopoietic cells comprise effector cells having one or more functional features that are not present in a counterpart primary T, NK, NKT, and/or B cell.

In some embodiments, the derivative cells comprise NK or T lineage cells. iPSC-derived NK or T lineage cells comprising IL, and optionally one or more of CD38^{-/-}. HLA deficiency, and exogenous polynucleotides encoding one or more of an additional cytokine signaling complex, a CAR, exogenous CD16, an antibody, a checkpoint inhibitor, an engager, and any other modality, as shown in Table 1, are useful for overcoming or reducing tumor microenvironment suppression associated with solid tumors. As discussed above, derivative CAR-T cells expressing hnCD16 have acquired ADCC, providing an additional mechanism for tumor killing in addition to CAR targeting. In some embodiments, the derivative cells comprise NK lineage cells. iPSC-derived NK cells comprising IL, and optionally one or more of CD38^{-/-}. HLA deficiency, and exogenous polynucleotides encoding one or more of an additional cytokine signaling complex, a CAR, exogenous CD16, an antibody, a checkpoint inhibitor, an engager, and any other modality, as shown in Table 1, have enhanced cytotoxicity, are effective in recruiting by-stander cells including T cells to infiltrate and kill tumor cells.

In some embodiments of effector cells comprising a signaling redirector receptor and optionally one or more of a CAR, an exogenous CD16 or variant thereof, CD38^{-/-}, a cytokine signaling complex, HLA modification, and any other modality, as shown in Table 1, derived from engineered iPSC, the cells are intact in HLA-II and still withstand allorejection by activated recipient T, B, and NK cells. In some embodiments, the iPSC and its derivative effector cells are intact in HLA-II and have synergistically increased persistence and/or survival in the presence of activated recipient T. B. and NK cells. In some embodiments, the iPSC and its derivative effector cells are useful for overcoming or reducing tumor microenvironment suppression associated with solid tumors. In some embodiments, when an anti-CD38 antibody is used with said derivative effector cells in a combination therapy, said cells have synergistically increased persistence, survival and effector function.

Also provided is an iPSC comprising a polynucleotide encoding a signaling redirector receptor (“SSR” in Table 1) and optionally one or more of a CAR, an exogenous CD16 or variant thereof, CD38^{-/-}, a cytokine signaling complex, HLA modification, and any other modality as shown in Table 1, wherein the iPSC is capable of directed differentiation to produce functional derivative hematopoietic cells optionally with HLA-E or HLA-G knockin, and knockout of one or both of CD54 and CD58, to overcome alloreactive NK cells.

Additionally provided is an iPSC comprising a cytokine signaling complex (IL), and optionally one or more of CD38. HLA deficiency, and exogenous polynucleotides encoding one or more of a CAR, exogenous CD16, an antibody, a checkpoint inhibitor, an engager, and any other modality, as shown in Table 1, where the iPSC further comprises a cytokine signaling complex comprising a polynucleotide encoding a full or partial peptide of a cytokine and/or a full or partial peptide of its receptor to enable further cytokine signaling contributing to cell survival, persistence and/or expansion, wherein the iPSC line is capable of hematopoietic differentiation to produce functional derivative effector cells having improved survival, persistency, expansion, and effector function. The exogenously introduced cytokine signaling(s) comprise the signaling of any one, two, or more of IL2, IL4, IL6, IL7, IL9, IL10, IL11, IL12, IL15, IL18, and IL21. In some embodiments, the introduced partial or full peptide of cytokine and/or its respective receptor comprises one or more of IL15RF and IL7RF. In some embodiments, the introduced partial or full peptide of cytokine and/or its respective receptor for cytokine signaling are expressed on the cell surface. In some embodiments, the exogenous cell surface cytokine and/or receptor comprised in the iPSC described herein, enables IL7 signaling. In some embodiments, the exogenous cell surface cytokine and/or receptor comprised in the iPSC described herein, enables IL15 signaling. In some embodiments of the iPSC described herein, the IL15 expression is through a construct as described herein. In some embodiments, the iPSC and its derivative effector cells are useful for overcoming or reducing tumor microenvironment suppression associated with solid tumors. In some embodiments, when an anti-CD38 antibody is used with said derivative effector cells in a combination therapy, said cells have synergistically increased persistence, survival and effector function.

As such, the present application provides iPSCs and their functional derivative hematopoietic cells, which comprise any one of the following genotypes in Table 1. In some embodiments, iPSCs and their functional derivative hematopoietic cells have a genotype comprising at least a signaling redirector receptor (“SRR” in Table 1) and a cytokine signaling complex (“IL” in Table 1). Further, when iPSCs and their functional derivative hematopoietic cells have a genotype comprising both CAR and IL, the CAR and IL may be comprised in a bi-cistronic expression cassette comprising a 2A sequence. As comparison, in some other embodiments, CAR and IL are in separate expression cassettes comprised in iPSCs and their functional derivative hematopoietic cells. In addition, “HLA” in Table 1 represents HLA-I and/or HLA-II modification which comprises: HLA-I knockout with or without HLA-II knockout, and optionally one or more of HLA-E or HLA-G knockin or overexpression, CD54 knockout and CD58 knockout. In some embodiments, iPSCs and their functional derivative hematopoietic cells have a genotype comprising at least a signaling redirector receptor and optionally a checkpoint inhibitor (“CI⁺ in Table 1), engager (“En⁺ in Table 1), or an antibody (“Ab⁺ in Table 1).

TABLE 1
Applicable Exemplary Genotypes of the Cells Provided:

| | | | | | | Ab⁺/Cl⁺ | | |
|---|---|---|---|---|---|---|---|---|
| | | | | | | (antibody/ | | |
| | | | | | | Check point | En⁺ | |
| SRR | IL | CAR | CD16^{exo} | CD38^{-/-} | HLA | inhibitor) | (engager) | Genotype |
| ✓ | | | | | | | | 1. SRR |
| ✓ | ✓ | | | | | | | 2. SRR IL |
| ✓ | | ✓ | | | | | | 3. SRR CAR |
| ✓ | | | ✓ | | | | | 4. SRR CD16^{exo} |
| ✓ | | | | ✓ | | | | 5. SRR CD38^{-/-} |
| ✓ | | | | | ✓ | | | 6. SRR HLA |
| ✓ | | | | | | ✓ | | 7. SRR Ab⁺ |
| | | | | | | | | 8. SRR Cl⁺ |
| ✓ | | | | | | | ✓ | 9. SRR En⁺ |
| ✓ | ✓ | ✓ | | | | | | 10. SRR IL CAR |
| ✓ | ✓ | | ✓ | | | | | 11. SRR IL CD16^{exo} |
| ✓ | ✓ | | | ✓ | | | | 12. SRR IL CD38^{-/-} |
| ✓ | ✓ | | | | ✓ | | | 13. SRR IL HLA |
| ✓ | ✓ | | | | | ✓ | | 14. SRR IL Ab⁺ |
| | | | | | | | | 15. SRR IL Cl⁺ |
| ✓ | ✓ | | | | | | ✓ | 16. SRR IL En⁺ |
| ✓ | | ✓ | ✓ | | | | | 17. SRR CAR CD16^{exo} |
| ✓ | | ✓ | | ✓ | | | | 18. SRR CAR CD38^{-/-} |
| ✓ | | ✓ | | | ✓ | | | 19. SRR CAR HLA |
| ✓ | | ✓ | | | | ✓ | | 20. SRR CAR Ab⁺ |
| | | | | | | | | 21. SRR CAR Cl⁺ |
| ✓ | | ✓ | | | | | ✓ | 22. SRR CAR En⁺ |
| ✓ | | | ✓ | ✓ | | | | 23. SRR CD16^{exo} CD38^{-/-} |
| ✓ | | | ✓ | | ✓ | | | 24. SRR CD16^{exo} HLA |
| ✓ | | | ✓ | | | ✓ | | 25. SRR CD16^{exo} Ab⁺ |
| | | | | | | | | 26. SRR CD16^{exo} Cl⁺ |
| ✓ | | | ✓ | | | | ✓ | 27. SRR CD16^{exo} En⁺ |
| ✓ | | | | ✓ | ✓ | | | 28. SRR CD38^{-/-} HLA |
| ✓ | | | | ✓ | | ✓ | | 29. SRR CD38^{-/-} Ab⁺ |
| | | | | | | | | 30. SRR CD38^{-/-} Cl⁺ |
| ✓ | | | | ✓ | | | ✓ | 31. SRR CD38^{-/-} En⁺ |
| ✓ | | | | | ✓ | ✓ | | 32. SRR HLA Ab⁺ |
| | | | | | | | | 33. SRR HLA Cl⁺ |
| ✓ | | | | | ✓ | | ✓ | 34. SRR HLA En⁺ |
| ✓ | | | | | | ✓ | ✓ | 35. SRR Ab⁺ En⁺ |
| | | | | | | | | 36. SRR Cl⁺ En⁺ |
| ✓ | ✓ | ✓ | ✓ | | | | | 37. SRR IL CAR CD16^{exo} |
| ✓ | ✓ | ✓ | | ✓ | | | | 38. SRR IL CAR CD38^{-/-} |
| ✓ | ✓ | ✓ | | | ✓ | | | 39. SRR IL CAR HLA |
| ✓ | ✓ | ✓ | | | | ✓ | | 40. SRR IL CAR Ab⁺ |
| | | | | | | | | 41. SRR IL CAR Cl⁺ |
| ✓ | ✓ | ✓ | | | | | ✓ | 42. SRR IL CAR En⁺ |
| ✓ | ✓ | | ✓ | ✓ | | | | 43. SRR IL CD16^{exo} CD38^{-/-} |
| ✓ | ✓ | | ✓ | | ✓ | | | 44. SRR IL CD16^{exo} HLA |
| ✓ | ✓ | | ✓ | | | ✓ | | 45. SRR IL CD16^{exo} Ab⁺ |
| | | | | | | | | 46. SRR IL CD16^{exo} Cl⁺ |
| ✓ | ✓ | | ✓ | | | | ✓ | 47. SRR IL CD16^{exo} En⁺ |
| ✓ | ✓ | | | ✓ | ✓ | | | 48. SRR IL CD38^{-/-} HLA |
| ✓ | ✓ | | | ✓ | | ✓ | | 49. SRR IL CD38^{-/-} Ab⁺ |
| | | | | | | | | 50. SRR IL CD38^{-/-} Cl⁺ |
| ✓ | ✓ | | | ✓ | | | ✓ | 51. SRR IL CD38^{-/-} En⁺ |
| ✓ | ✓ | | | | ✓ | ✓ | | 52. SRR IL HLA Ab⁺ |
| | | | | | | | | 53. SRR IL HLA Cl⁺ |
| ✓ | ✓ | | | | ✓ | | ✓ | 54. SRR IL HLA En⁺ |
| ✓ | ✓ | | | | | ✓ | ✓ | 55. SRR IL Ab⁺ En⁺ |
| | | | | | | | | 56. SRR IL Cl⁺ En⁺ |
| ✓ | | ✓ | ✓ | ✓ | | | | 57. SRR CAR CD16^{exo} CD38^{-/-} |
| ✓ | | ✓ | ✓ | | ✓ | | | 58. SRR CAR CD16^{exo} CD38^{-/-} |
| ✓ | | ✓ | ✓ | | | ✓ | | 59. SRR CAR CD16^{exo} Ab⁺ |
| | | | | | | | | 60. SRR CAR CD16^{exo} Cl⁺ |
| ✓ | | ✓ | ✓ | | | | ✓ | 61. SRR CAR CD16^{exo} En⁺ |
| ✓ | | ✓ | | ✓ | ✓ | | | 62. SRR CAR CD38^{-/-} HLA |
| ✓ | | ✓ | | ✓ | | ✓ | | 63. SRR CAR CD38^{-/-} Ab⁺ |
| | | | | | | | | 64. SRR CAR CD38^{-/-} Cl⁺ |
| ✓ | | ✓ | | ✓ | | | ✓ | 65. SRR CAR CD38^{-/-} En⁺ |
| ✓ | | ✓ | | | ✓ | ✓ | | 66. SRR CAR HLA Ab⁺ |
| | | | | | | | | 67. SRR CAR HLA Cl⁺ |
| ✓ | | ✓ | | | ✓ | | ✓ | 68. SRR CAR HLA En⁺ |
| ✓ | | ✓ | | | | ✓ | ✓ | 69. SRR CAR Ab⁺ En⁺ |
| | | | | | | | | 70. SRR CAR Cl⁺ En⁺ |
| ✓ | | | ✓ | ✓ | ✓ | | | 71. SRR CD16^{exo} CD38^{-/-} HLA |
| ✓ | | | ✓ | ✓ | | ✓ | | 72. SRR CD16^{exo} CD38^{-/-} Ab⁺ |
| | | | | | | | | 73. SRR CD16^{exo} CD38^{-/-} Cl⁺ |
| ✓ | | | ✓ | ✓ | | | ✓ | 74. SRR CD16^{exo} CD38^{-/-} En⁺ |
| ✓ | | | | ✓ | ✓ | ✓ | | 75. SRR CD38^{-/-} HLA Ab⁺ |
| | | | | | | | | 76. SRR CD38^{-/-} HLA Cl⁺ |
| ✓ | | | | ✓ | ✓ | | ✓ | 77. SRR CD38^{-/-} HLA En⁺ |
| ✓ | | | | | ✓ | ✓ | ✓ | 78. SRR HLA Ab⁺ En⁺ |
| | | | | | | | | 79. SRR HLA Cl⁺ En⁺ |
| ✓ | ✓ | ✓ | ✓ | ✓ | | | | 80. SRR IL CAR CD16^{exo} CD38^{-/-} |
| ✓ | ✓ | ✓ | ✓ | | ✓ | | | 81. SRR IL CAR CD16^{exo} HLA |
| ✓ | ✓ | ✓ | ✓ | | | ✓ | | 82. SRR IL CAR CD16^{exo} Ab⁺ |
| | | | | | | | | 83. SRR IL CAR CD16^{exo} Cl⁺ |
| ✓ | ✓ | ✓ | ✓ | | | | ✓ | 84. SRR IL CAR CD16^{exo} En⁺ |
| ✓ | ✓ | ✓ | | ✓ | ✓ | | | 85. SRR IL CAR CD38^{-/-} HLA |
| ✓ | ✓ | ✓ | | ✓ | | ✓ | | 86. SRR IL CAR CD38^{-/-} Ab⁺ |
| | | | | | | | | 87. SRR IL CAR CD38^{-/-} Cl⁺ |
| ✓ | ✓ | ✓ | | ✓ | | | ✓ | 88. SRR IL CAR CD38^{-/-} En⁺ |
| ✓ | ✓ | ✓ | | | ✓ | ✓ | | 89. SRR IL CAR HLA Ab⁺ |
| | | | | | | | | 90. SRR IL CAR HLA Cl⁺ |
| ✓ | ✓ | ✓ | | | ✓ | | ✓ | 91. SRR IL CAR HLA En⁺ |
| ✓ | ✓ | ✓ | | | | ✓ | ✓ | 92. SRR IL CAR Ab⁺ En⁺ |
| | | | | | | | | 93. SRR IL CAR Cl⁺ En⁺ |
| ✓ | ✓ | | ✓ | ✓ | ✓ | | | 94. SRR IL CD16^{exo} CD38^{-/-} HLA |
| ✓ | ✓ | | ✓ | ✓ | | ✓ | | 95. SRR IL CD16^{exo} CD38^{-/-} Ab⁺ |
| | | | | | | | | 96. SRR IL CD16^{exo} CD38^{-/-} Cl⁺ |
| ✓ | ✓ | | ✓ | ✓ | | | ✓ | 97. SRR IL CD16^{exo} CD38^{-/-} En⁺ |
| ✓ | ✓ | | ✓ | | ✓ | ✓ | | 98. SRR IL CD16^{exo} HLA Ab⁺ |
| | | | | | | | | 99. SRR IL CD16^{exo} HLA Cl⁺ |
| ✓ | ✓ | | ✓ | | ✓ | | ✓ | 100. SRR IL CD16^{exo} HLA En⁺ |
| ✓ | ✓ | | ✓ | | | ✓ | ✓ | 101. SRR IL CD16^{exo} Ab⁺ En⁺ |
| | | | | | | | | 102. SRR IL CD16^{exo} Cl⁺ En⁺ |
| ✓ | ✓ | | | ✓ | ✓ | ✓ | | 103. SRR IL CD38^{-/-} HLA Ab⁺ |
| | | | | | | | | 104. SRR IL CD38^{-/-} HLA Cl⁺ |
| ✓ | ✓ | | | ✓ | ✓ | | ✓ | 105. SRR IL CD38^{-/-} HLA En⁺ |
| ✓ | ✓ | | | | ✓ | ✓ | ✓ | 106. SRR IL HLA Ab+ En+ |
| | | | | | | | | 107. SRR IL HLA Cl⁺ En⁺ |
| ✓ | | ✓ | ✓ | ✓ | ✓ | | | 108. SRR CAR CD16^{exo} CD38^{-/-} HLA |
| ✓ | | ✓ | ✓ | ✓ | | ✓ | | 109. SRR CAR CD16^{exo} CD38^{-/-} HLA Ab⁺ |
| | | | | | | | | 110. SRR CAR CD16^{exo} CD38^{-/-} HLA Cl⁺ |
| ✓ | | ✓ | ✓ | ✓ | | | ✓ | 111. SRR CAR CD16^{exo} CD38^{-/-} HLA En⁺ |
| ✓ | | | ✓ | ✓ | ✓ | ✓ | | 112. SRR CD16^{exo} CD38^{-/-} HLA Ab⁺ |
| | | | | | | | | 113. SRR CD16^{exo} CD38^{-/-} HLA Cl⁺ |
| ✓ | | | ✓ | ✓ | ✓ | | ✓ | 114. SRR CD16^{exo} CD38^{-/-} HLA En⁺ |
| ✓ | | | | ✓ | ✓ | ✓ | ✓ | 115. SRR CD38^{-/-} HLA Ab⁺ En⁺ |
| | | | | | | | | 116. SRR CD38^{-/-} HLA Cl⁺ En⁺ |
| ✓ | ✓ | ✓ | ✓ | ✓ | ✓ | | | 117. SRR IL CAR CD16^{exo} CD38^{-/-} HLA |
| ✓ | ✓ | ✓ | ✓ | ✓ | | ✓ | | 118. SRR IL CAR CD16^{exo} CD38^{-/-} Ab⁺ |
| | | | | | | | | 119. SRR IL CAR CD16^{exo} CD38^{-/-} Cl⁺ |
| ✓ | ✓ | ✓ | ✓ | ✓ | | | ✓ | 120. SRR IL CAR CD16^{exo} CD38^{-/-} En⁺ |
| ✓ | ✓ | ✓ | ✓ | | ✓ | ✓ | | 121. SRR IL CAR CD16^{exo} HLA Ab⁺ |
| | | | | | | | | 122. SRR IL CAR CD16^{exo} HLA Cl⁺ |
| ✓ | ✓ | ✓ | ✓ | | ✓ | | ✓ | 123. SRR IL CAR CD16^{exo} HLA En⁺ |
| ✓ | ✓ | ✓ | ✓ | | | ✓ | ✓ | 124. SRR IL CAR CD16^{exo} Ab⁺ En⁺ |
| | | | | | | | | 125. SRR IL CAR CD16^{exo} Cl⁺ En⁺ |
| ✓ | ✓ | ✓ | | ✓ | ✓ | ✓ | | 126. SRR IL CAR CD38^{-/-} HLA Ab⁺ |
| | | | | | | | | 127. SRR IL CAR CD38^{-/-} HLA Cl⁺ |
| ✓ | ✓ | ✓ | | ✓ | ✓ | | ✓ | 128. SRR IL CAR CD38^{-/-} HLA En⁺ |
| ✓ | ✓ | ✓ | | | ✓ | ✓ | ✓ | 129. SRR IL CAR HLA Ab⁺ En⁺ |
| | | | | | | | | 130. SRR IL CAR HLA Cl⁺ En⁺ |
| ✓ | ✓ | | ✓ | ✓ | ✓ | ✓ | | 131. SRR IL CD16^{exo} CD38^{-/-} HLA Ab⁺ |
| | | | | | | | | 132. SRR IL CD16^{exo} CD38^{-/-} HLA Cl⁺ |
| ✓ | ✓ | | ✓ | ✓ | ✓ | | ✓ | 133. SRR IL CD16^{exo} CD38^{-/-} HLA En⁺ |
| ✓ | ✓ | | ✓ | | ✓ | ✓ | ✓ | 134. SRR IL CD16^{exo} HLA Ab⁺ En⁺ |
| | | | | | | | | 135. SRR IL CD16^{exo} HLA Cl⁺ En⁺ |
| ✓ | ✓ | | ✓ | ✓ | | ✓ | ✓ | 136. SRR IL CD16^{exo} CD38^{-/-} Ab⁺ En⁺ |
| | | | | | | | | 137. SRR IL CD16^{exo} CD38^{-/-} Cl⁺ En⁺ |
| ✓ | ✓ | | | ✓ | ✓ | ✓ | ✓ | 138. SRR IL CD38^{-/-} HLA Ab⁺ En⁺ |
| | | | | | | | | 139. SRR IL CD38^{-/-} HLA Cl⁺ En⁺ |
| ✓ | ✓ | ✓ | ✓ | ✓ | ✓ | ✓ | | 140. SRR IL CAR CD16^{exo} CD38^{-/-} HLA Ab⁺ |
| | | | | | | | | 141. SRR IL CAR CD16^{exo} CD38^{-/-} HLA Cl⁺ |
| ✓ | | ✓ | ✓ | ✓ | ✓ | ✓ | | 142. SRR CAR CD16^{exo} CD38^{-/-} HLA Ab⁺ |
| | | | | | | | | 143. SRR CAR CD16^{exo} CD38^{-/-} HLA Cl⁺ |
| ✓ | | ✓ | ✓ | ✓ | ✓ | | ✓ | 144. SRR CAR CD16^{exo} CD38^{-/-} HLA En⁺ |
| ✓ | | | ✓ | ✓ | ✓ | ✓ | ✓ | 145. SRR CD16^{exo} CD38^{-/-} HLA Ab⁺ En⁺ |
| | | | | | | | | 146. SRR CD16^{exo} CD38^{-/-} HLA Cl⁺ En⁺ |
| ✓ | ✓ | ✓ | ✓ | ✓ | ✓ | ✓ | | 147. SRR IL CAR CD16^{exo} CD38^{-/-} HLA Ab⁺ |
| | | | | | | | | 148. SRR IL CAR CD16^{exo} CD38^{-/-} HLA Cl⁺ |
| ✓ | ✓ | ✓ | ✓ | ✓ | ✓ | | ✓ | 149. SRR IL CAR CD16^{exo} CD38^{-/-} HLA En⁺ |
| ✓ | ✓ | ✓ | ✓ | ✓ | | ✓ | ✓ | 150. SRR IL CAR CD16^{exo} CD38^{-/-} Ab⁺ En⁺ |
| | | | | | | | | 151. SRR IL CAR CD16^{exo} CD38^{-/-} Cl⁺ En⁺ |
| ✓ | ✓ | ✓ | ✓ | | ✓ | ✓ | ✓ | 152. SRR IL CAR CD16^{exo} HLA Ab⁺ En⁺ |
| | | | | | | | | 153. SRR IL CAR CD16^{exo} HLA Cl⁺ En⁺ |
| ✓ | ✓ | ✓ | | ✓ | ✓ | ✓ | ✓ | 154. SRR IL CAR CD38^{-/-} HLA Ab⁺ En⁺ |
| | | | | | | | | 155. SRR IL CAR CD38^{-/-} HLA Cl⁺ En⁺ |
| ✓ | ✓ | | ✓ | ✓ | ✓ | ✓ | ✓ | 156. SRR IL CD16^{exo} CD38^{-/-} HLA Ab⁺ En⁺ |
| | | | | | | | | 157. SRR IL CD16^{exo} CD38^{-/-} HLA Cl⁺ En⁺ |
| ✓ | | ✓ | ✓ | ✓ | ✓ | ✓ | ✓ | 158. SRR CAR CD16^{exo} CD38^{-/-} HLA Ab⁺En⁺ |
| | | | | | | | | 159. SRR CAR CD16^{exo} CD38^{-/-} HLA Cl⁺En⁺ |
| ✓ | ✓ | ✓ | ✓ | ✓ | ✓ | ✓ | ✓ | 160. SRR IL CAR CD16^{exo} CD38^{-/-} HLA Ab⁺ En⁺ |
| | | | | | | | | 161. SRR IL CAR CD16^{exo} CD38^{-/-} HLA Cl⁺ En⁺ |

### 7. Additional Modifications

In some embodiments, the genetically modified iPSC and the derivative cells thereof further comprise one or more of the following genetically modified modalities: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments, the iPSC, and its derivative effector cells comprising any one of the genotypes in Table 1 may additionally comprise deletion or disruption of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, HLA-G, 4-1BBL, CD3, CD4, CD8, CD47, CD113, CD131, CD137, CD80, PDL1, A_{2A}R, TCR, Fc receptor, and surface triggering receptor for coupling with bi-, multi-specific or universal engagers.

### II. Methods for Targeted Genome Editing at Selected Locus in iPSCs

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. Therefore, targeted editing may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.” In some situations, the targeted integration site is intended to be within a coding region of a selected gene, and thus the targeted integration could disrupt the gene expression, resulting in simultaneous knock-in and knock-out (KI/KO) in one single editing step.

Inserting one or more transgenes at a selected position in a gene locus of interest (GOI) to knock-out the gene at the same time can be achieved. Gene loci suitable for simultaneous knock-in and knockout (KI/KO) include, but are not limited to, B2M, TAP1. TAP2, tapasin, NLRC5, CIITA, RFXANK, RFX5, RFXAP, TCRα or TCRβ constant region, NKG2A, NKG2D, CD38, CD25, CD69, CD71, CD44, CD58, CD54, CD56, CIS, CBL-B, SOCS2, PD1, CTLA4, LAG3, TIM3, and TIGIT. With respective site-specific targeting homology arms for position-selective insertion, it allows the transgene(s) to express either under an endogenous promoter at the site or under an exogenous promoter comprised in the construct. When two or more transgenes are to be inserted at a selected location in CD38 locus, a linker sequence, for example, a 2A linker or IRES, is placed between any two transgenes. The 2A linker encodes a self-cleaving peptide derived from, e.g., FMDV, ERAV, PTV-I, or TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. In various embodiments, the exogenous promoter may be CAG, or other constitutive, inducible, temporal-, tissue-, or cell type-specific promoters including, but not limited to CMV, EF1α, PGK, and UBC.

Available endonucleases capable of introducing specific and targeted DSBs include, but are not limited to, zinc-finger nucleases (ZFN), transcription activator-like effector nucleases (TALEN), RNA-guided CRISPR (Clustered Regular Interspaced Short Palindromic Repeats) systems. Additionally, the DICE (dual integrase cassette exchange) system utilizing phiC31 and Bxb1 integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C₂H₂ zinc fingers, C₃H zinc fingers, and C₄ zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496, the complete disclosures of which are incorporated herein by reference. A“selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain”, it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Pub. No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spo11 nuclease, a polypeptide comprising a Spo11 polypeptide having nuclease activity fused to a DNA binding domain, e.g., a zinc finger DNA binding domain, a TAL effector DNA binding domain, etc. that has specificity for a DNA sequence of interest.

Additional examples of targeted nucleases suitable for embodiments of the present invention include, but not limited to Bxb1, phiC31, R4, PhiBT1, and Wp/SPBctFP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases: homing endonucleases, and the like.

Using Cas9 as an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction.

DICE mediated insertion uses a pair of recombinases, for example, phiC31 and Bxb1, to provide unidirectional integration of an exogenous DNA that is tightly restricted to each enzymes' own small attB and attP recognition sites. Because these target att sites that are not naturally present in mammalian genomes, they must be first introduced into the genome, at the desired integration site. See, for example, U.S. Pub. No. 2015/0140665, the disclosure of which is incorporated herein by reference.

One aspect of the present invention provides a construct comprising one or more exogenous polynucleotides for targeted genome integration. In one embodiment, the construct further comprises a pair of homologous arms specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Suitable sites for human genome editing, or specifically, targeted integration, include, but are not limited to, the adeno-associated virus site 1 (AAVS1), the chemokine (CC motif) receptor 5 (CCR5) gene locus and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In some embodiments, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, one aspect of the present invention provides a method of targeted integration in a selected locus including genome safe harbor or a preselected locus known or proven to be safe and well-regulated for continuous or temporal gene expression such as, in the case of a T cell, a constant region of a T Cell Receptor (TCR). In one embodiment, the genome safe harbor for the method of targeted integration comprises one or more desired integration site comprising AAVS1, CCR5, ROSA26, collagen, HTRP, H11, GAPDH, TCR or RUNX1, or other loci meeting the criteria of a genome safe harbor. In one embodiment, the method of targeted integration in a cell comprises introducing a construct comprising one or more exogenous polynucleotides to the cell, and introducing a construct comprising a pair of homologous arms specific to a desired integration site and one or more exogenous sequence, to enable site specific homologous recombination by the cell host enzymatic machinery, wherein the desired integration site comprises AAVS1, CCR5, ROSA26, collagen, HTRP, H11, GAPDH, TCR or RUNX1, or other loci meeting the criteria of a genome safe harbor. Additional integration sites include an endogenous gene locus intended for disruption, such as reduction or knockout, which comprises B2M, TAP1, TAP2, tapasin, NLRC5, CIITA, RFXANK, RFX5, RFXAP, TCRα or TCRβ constant region (TRAC or TRBC), NKG2A, NKG2D, CD38, CD25, CD69, CD71, CD44, CD54, CD56, CD58, OX40, 4-1BB, CIS, CBL-B, SOCS2, PD1. CTLA4, LAG3, TIM3, or TIGIT.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3, or TIGIT In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region. NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, ROSA26, collagen, HTRP H11, GAPDH, RUNX1, B2M, TAP1, TAP2, tapasin, NLRC5, CIITA. RFXANK, RFX5, RFXAP, TCRα or TCRβ constant region. NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT

Further, as provided herein, the above method for targeted integration in a safe harbor is used to insert any polynucleotide of interest, for example, polynucleotides encoding safety switch proteins, targeting modality, receptors, signaling molecules, transcription factors, pharmaceutically active proteins and peptides, drug target candidates, and proteins promoting engraftment, trafficking, homing, viability, self-renewal, persistence, and/or survival of stem cells and/or progenitor cells. In some other embodiments, the construct comprising one or more exogenous polynucleotides further comprises one or more marker genes. In one embodiment, the exogenous polynucleotide in a construct of the invention is a suicide gene encoding a safety switch protein. Suitable suicide gene systems for induced cell death include, but not limited to Caspase 9 (or caspase 3 or 7) and AP1903; thymidine kinase (TK) and ganciclovir (GCV); cytosine deaminase (CD) and 5-fluorocytosine (5-FC). Additionally, some suicide gene systems are cell type specific, for example, the genetic modification of T lymphocytes with the B-cell molecule CD20 allows their elimination upon administration of mAb Rituximab. Further, modified EGFR containing epitope recognized by cetuximab can be used to deplete genetically engineered cells when the cells are exposed to cetuximab. As such, one aspect of the invention provides a method of targeted integration of one or more suicide genes encoding safety switch proteins selected from caspase 9 (caspase 3 or 7), thymidine kinase, cytosine deaminase, modified EGFR, and B cell CD20.

In some embodiments, one or more exogenous polynucleotides integrated by the method described herein are driven by operatively-linked exogenous promoters comprised in the construct for targeted integration. The promoters may be inducible, or constructive, and may be temporal-, tissue- or cell type-specific. Suitable constructive promoters for methods of the invention include, but not limited to, cytomegalovirus (CMV), elongation factor 1α (EF1α), phosphoglycerate kinase (PGK), hybrid CMV enhancer/chicken R-actin (CAG) and ubiquitin C (UBC) promoters. In one embodiment, the exogenous promoter is CAG.

The exogenous polynucleotides integrated by the method described herein may be driven by endogenous promoters in the host genome, at the integration site. In one embodiment, the method described herein is used for targeted integration of one or more exogenous polynucleotides at AAVS1 locus in the genome of a cell. In one embodiment, at least one integrated polynucleotide is driven by the endogenous AAVS1 promoter. In another embodiment, the method described herein is used for targeted integration at ROSA26 locus in the genome of a cell. In one embodiment, at least one integrated polynucleotide is driven by the endogenous ROSA26 promoter. In still another embodiment, the method described herein is used for targeted integration at H11 locus in the genome of a cell. In one embodiment, at least one integrated polynucleotide is driven by the endogenous H11 promoter. In another embodiment, the method described herein is used for targeted integration at collagen locus in the genome of a cell. In one embodiment, at least one integrated polynucleotide is driven by the endogenous collagen promoter. In still another embodiment, the method described herein is used for targeted integration at HTRP locus in the genome of a cell. In one embodiment, at least one integrated polynucleotide is driven by the endogenous HTRP promoter. Theoretically, only correct insertions at the desired location would enable gene expression of an exogenous gene driven by an endogenous promoter.

In some embodiments, the one or more exogenous polynucleotides comprised in the construct for the methods of targeted integration are driven by one promoter. In some embodiments, the construct comprises one or more linker sequences between two adjacent polynucleotides driven by the same promoter to provide greater physical separation between the moieties and maximize the accessibility to enzymatic machinery. The linker peptide of the linker sequences may consist of amino acids selected to make the physical separation between the moieties (exogenous polynucleotides, and/or the protein or peptide encoded therefrom) more flexible or more rigid depending on the relevant function. The linker sequence may be cleavable by a protease or cleavable chemically to yield separate moieties. Examples of enzymatic cleavage sites in the linker include sites for cleavage by a proteolytic enzyme, such as enterokinase. Factor Xa, trypsin, collagenase, and thrombin. In some embodiments, the protease is one which is produced naturally by the host or it is exogenously introduced. Alternatively, the cleavage site in the linker may be a site capable of being cleaved upon exposure to a selected chemical, e.g., cyanogen bromide, hydroxylamine, or low pH. The optional linker sequence may serve a purpose other than the provision of a cleavage site. The linker sequence should allow effective positioning of the moiety with respect to another adjacent moiety for the moieties to function properly. The linker may also be a simple amino acid sequence of a sufficient length to prevent any steric hindrance between the moieties. In addition, the linker sequence may provide for post-translational modification including, but not limited to, e.g., phosphorylation sites, biotinylation sites, sulfation sites, γ-carboxylation sites, and the like. In some embodiments, the linker sequence is flexible so as not to hold the biologically active peptide in a single undesired conformation. The linker may be predominantly comprised of amino acids with small side chains, such as glycine, alanine, and seine, to provide for flexibility. In some embodiments about 80 to 90 percent or greater of the linker sequence comprises glycine, alanine, or serine residues, particularly glycine and serine residues. In several embodiments, a G4S linker peptide separates the end-processing and endonuclease domains of the fusion protein. In other embodiments, a 2A linker sequence allows for two separate proteins to be produced from a single translation. Suitable linker sequences can be readily identified empirically. Additionally, suitable size and sequences of linker sequences also can be determined by conventional computer modeling techniques. In one embodiment, the linker sequence encodes a self-cleaving peptide. In one embodiment, the self-cleaving peptide is 2A. In some other embodiments, the linker sequence provides an Internal Ribosome Entry Sequence (IRES). In some embodiments, any two consecutive linker sequences are different.

The method of introducing into cells a construct comprising exogenous polynucleotides for targeted integration can be achieved using a method of gene transfer to cells known per se. In one embodiment, the construct comprises backbones of viral vectors such as adenovirus vector, adeno-associated virus vector, retrovirus vector, lentivirus vector, Sendai virus vector. In some embodiments, the plasmid vectors are used for delivering and/or expressing the exogenous polynucleotides to target cells (e.g., pAl-11, pXTl, pRc/CMV, pRc/RSV, pcDNAI/Neo) and the like. In some other embodiments, the episomal vector is used to deliver the exogenous polynucleotide to target cells. In some embodiments, recombinant adeno-associated viruses (rAAV) can be used for genetic engineering to introduce insertions, deletions or substitutions through homologous recombination. Unlike lentiviruses, rAAVs do not integrate into the host genome. In addition, episomal rAAV vectors mediate homology-directed gene targeting at much higher rates compared to transfection of conventional targeting plasmids. In some embodiments, an AAV6 or AAV2 vector is used to introduce insertions, deletions or substitutions in a target site in the genome of iPSCs. In some embodiments, the genomically modified iPSCs and their derivative cells obtained using the methods and compositions described herein comprise at least one genotype listed in Table 1.

### III. Method of Obtaining and Maintaining Genome-Engineered iPSCs

In various embodiments, the present invention provides methods of obtaining and maintaining genome-engineered iPSCs comprising one or more targeted edits (e.g., multiplex genomic engineering) at one or more desired sites, wherein the one or more targeted edits remain intact and functional in expanded genome-engineered iPSCs or the iPSC-derived non-pluripotent cells at the respective selected editing sites. The targeted editing introduces into the genome of the iPSC, and derivative cells thereof, insertions, deletions, and/or substitutions (i.e., targeted integration and/or in/dels at selected sites). In comparison to direct engineering of patient-sourced, peripheral blood originated primary effector cells, the many benefits of obtaining genomically-engineered derivative cells through editing and differentiating iPSC as provided herein include, but are not limited to: unlimited source for engineered effector cells; no need for repeated manipulation of the effector cells, especially when multiple engineered modalities are involved; the obtained effector cells are rejuvenated for having elongated telomere and experiencing less exhaustion: the effector cell population is homogeneous in terms of editing site, copy number, and void of allelic variation, random mutations and expression variegation, largely due to the enabled clonal selection in engineered iPSCs as provided herein.

In some embodiments, the genome-engineered iPSCs comprising one or more targeted edits at one or more selected sites are maintained, passaged and expanded as single cells for an extended period in cell maintenance culture medium (FMM), wherein the iPSCs retain the targeted editing and functional modification at the selected site(s). The iPSCs cultured in FMM have been shown to continue to maintain their undifferentiated, and ground or naïve, profile; provide genomic stability without the need for culture cleaning or selection; and readily to give rise to all three somatic lineages, in vitro differentiation via embryoid bodies or monolayer (without formation of embryoid bodies); and by in vivo differentiation via teratoma formation. See, for example, International Pub. No. WO2015/134652, the disclosure of which is incorporated herein by reference.

In some embodiments, the genome-engineered iPSCs comprising one or more targeted integrations and/or in/dels are maintained, passaged and expanded in a medium (FMM) comprising a MEK inhibitor, a GSK3 inhibitor, and a ROCK inhibitor, and free of, or essentially free of, TGFβ receptor/ALK5 inhibitors, wherein the iPSCs retain the intact and functional targeted edits at the selected sites.

Another aspect of the invention provides a method of generating genome-engineered iPSCs through targeted editing of iPSCs; or through first generating genome-engineered non-pluripotent cells by targeted editing, and then reprogramming the selected/isolated genome-engineered non-pluripotent cells to obtain iPSCs comprising the same targeted editing as the non-pluripotent cells. A further aspect of the invention provides genome-engineering non-pluripotent cells which are concurrently undergoing reprogramming by introducing targeted integration and/or targeted in/dels to the cells, wherein the contacted non-pluripotent cells are under sufficient conditions for reprogramming, and wherein the conditions for reprogramming comprise contacting non-pluripotent cells with one or more reprogramming factors and small molecules. In various embodiments of the method for concurrent genome-engineering and reprogramming, the targeted integrations and/or targeted in/dels may be introduced to the non-pluripotent cells prior to, or essentially concomitantly with, initiating reprogramming by contacting the non-pluripotent cells with one or more reprogramming factors and optionally one or more small molecules.

In some embodiments, to concurrently genome-engineer and reprogram non-pluripotent cells, the targeted integrations and/or in/dels may also be introduced to the non-pluripotent cells after the multi-day process of reprogramming is initiated by contacting the non-pluripotent cells with one or more reprogramming factors and small molecules, and wherein the vectors carrying the constructs are introduced before the reprogramming cells present stable expression of one or more endogenous pluripotent genes including, but not limited to, SSEA4, Tra181 and CD30.

In some embodiments, the reprogramming is initiated by contacting the non-pluripotent cells with at least one reprogramming factor, and optionally a combination of a TGFβ receptor/ALK inhibitor, a MEK inhibitor, a GSK3 inhibitor and a ROCK inhibitor. In some embodiments, the genome-engineered iPSCs produced through any methods above are further maintained and expanded using a mixture comprising a combination of a MEK inhibitor, a GSK3 inhibitor and a ROCK inhibitor.

In some embodiments of the method of generating genome-engineered iPSCs, the method comprises: genomically engineering an iPSC by introducing one or more targeted integrations and/or in/dels into iPSCs to obtain genome-engineered iPSCs having a genotype provided herein. Alternatively, the method of generating genome-engineered iPSCs comprises: (a) introducing one or more targeted edits into non-pluripotent cells to obtain genome-engineered non-pluripotent cells comprising targeted integrations and/or in/dels at selected sites, and (b) contacting the genome-engineered non-pluripotent cells with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted integrations and/or in/dels at selected sites. Alternatively, the method of generating genome-engineered iPSCs comprises: (a) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate the reprogramming of the non-pluripotent cells; (b) introducing one or more targeted integrations and/or in/dels into the reprogramming non-pluripotent cells for genome-engineering; and (c) obtaining clonal genome-engineered iPSCs comprising the targeted integrations and/or in/dels at selected sites. Any of the above methods may further comprise single cell sorting of the genome-engineered iPSCs to obtain a clonal iPSC. Through clonal expansion of the genome-engineered iPSCs, a master cell bank is generated to comprise single cell sorted and expanded clonal engineered iPSCs having at least one phenotype as provided herein. The master cell bank is subsequently cryopreserved, providing a platform for additional iPSC engineering and a renewable source for manufacturing off-the-shelf, engineered, homogeneous cell therapy products, which are well-defined and uniform in composition, and can be mass produced at significant scale in a cost-effective manner.

The reprogramming factors are selected from the group consisting of OCT4, SOX2, NANOG, KLF4, LIN28, C-MYC, ECAT1, UTF1, ESRRB, SV40LT, HESRG, CDH1, TDGF1, DPPA4, DNMT3B, ZIC3, LITD1, and any combinations thereof as disclosed in International Pub. Nos. WO2015/134652 and WO 2017/066634, the disclosures of which are incorporated herein by reference. The one or more reprogramming factors may be in the form of a polypeptide. The reprogramming factors may also be in the form of polynucleotides encoding the reprogramming factors, and thus may be introduced to the non-pluripotent cells by vectors such as, a retrovirus, a Sendai virus, an adenovirus, an episome, a plasmid, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the one or more polynucleotides introduced by a combination of plasmids. See, for example, International Pub. No. WO2019/075057A1, the disclosure of which is incorporated herein by reference.

In some embodiments, the non-pluripotent cells are transfected with multiple constructs comprising different exogenous polynucleotides and/or different promoters by multiple vectors for targeted integration at the same or different selected sites. These exogenous polynucleotides may comprise a suicide gene, or gene encoding targeting modalities, receptors, signaling molecules, transcription factors, pharmaceutically active proteins and peptides, drug target candidates, or a gene encoding a protein promoting engraftment, trafficking, homing, viability, self-renewal, persistence, and/or survival of the iPSCs or derivative cells thereof. In some embodiments, the exogenous polynucleotides encode RNA, including but not limited to siRNA, shRNA, miRNA and antisense nucleic acids. These exogenous polynucleotides may be driven by one or more promoters selected from the group consisting of constitutive promoters, inducible promoters, temporal-specific promoters, and tissue or cell type specific promoters. Accordingly, the polynucleotides are expressible when under conditions that activate the promoter, for example, in the presence of an inducing agent or in a particular differentiated cell type. In some embodiments, the polynucleotides are expressed in iPSCs and/or in cells differentiated from the iPSCs. In one embodiment, one or more suicide genes are driven by a constitutive promoter, for example Capase-9 driven by CAG. These constructs comprising different exogenous polynucleotides and/or different promoters can be transfected to non-pluripotent cells either simultaneously or consecutively. The non-pluripotent cells subjected to targeted integration of multiple constructs can simultaneously contact the one or more reprogramming factors to initiate the reprogramming concurrently with the genomic engineering, thereby obtaining genome-engineered iPSCs comprising multiple targeted integrations in the same pool of cells. As such, this robust method enables a concurrent reprogramming and engineering strategy to derive a clonal genomically-engineered iPSC with multiple modalities integrated into one or more selected target sites.

### IV. A Method of Obtaining Genetically-Engineered Effector Cells by Differentiating Genome-Engineered iPSC

A further aspect of the present invention provides a method of in vivo differentiation of genome-engineered iPSCs by teratoma formation, wherein the differentiated cells derived in vivo from the genome-engineered iPSCs retain the intact and functional targeted edits including targeted integration(s) and/or in/dels at the desired site(s). In some embodiments, the differentiated cells derived in vivo from the genome-engineered iPSCs via teratoma formation comprise one or more inducible suicide genes integrated at one or more desired sites comprising AAVS1, CCR5, ROSA26, collagen, HTRP H11, beta-2 microglobulin, CD38, GAPDH, TCR or RUNX1, or other loci meeting the criteria of a genome safe harbor. In some other embodiments, the differentiated cells derived in vivo from the genome-engineered iPSCs via teratoma formation comprise polynucleotides encoding targeting modalities, or encoding proteins promoting trafficking, homing, viability, self-renewal, persistence, and/or survival of stem cells and/or progenitor cells. In some embodiments, the differentiated cells derived in vivo from the genome-engineered iPSCs via teratoma formation comprising one or more inducible suicide genes further comprise one or more in/dels in endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In some embodiments, the in/del is comprised in one or more endogenous genes including, but not limited to, AAVS1, CCR5, ROSA26, collagen, HTRP, H11, GAPDH, RUNX1, B2M, TAP1. TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1. CTLA4, LAG3, TIM3, and TIGIT.

In some embodiments, the genome-engineered iPSCs comprising one or more genetic modifications as provided herein are used to derive hematopoietic cell lineages or any other specific cell types in vitro, wherein the derived non-pluripotent cells retain the functional genetic modifications including targeted editing at the selected site(s). In some embodiments, the genome-engineered iPSCs used to derive hematopoietic cell lineages or any other specific cell types in vitro are master cell bank cells that are cryopreserved and thawed right before their usage. In one embodiment, the genome-engineered iPSC-derived cells include, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T cells. NKT cells, NK cells, B cells, neutrophils, dendritic cells, and macrophages, wherein the cells derived from the genome-engineered iPSCs retain the functional genetic modifications including targeted editing at the desired site(s).

Applicable differentiation methods and compositions for obtaining iPSC-derived hematopoietic cell lineages include those depicted in, for example, International Pub. No. WO2017/078807, the disclosure of which is incorporated herein by reference. As provided, the methods and compositions for generating hematopoietic cell lineages are through definitive hemogenic endothelium (HE) derived from pluripotent stem cells, including iPSCs under serum-free, feeder-free, and/or stromal-free conditions and in a scalable and monolayer culturing platform without the need of EB formation. Cells that may be differentiated according to the provided methods range from pluripotent stem cells, to progenitor cells that are committed to particular terminally differentiated cells and transdifferentiated cells, and to cells of various lineages directly transitioned to hematopoietic fate without going through a pluripotent intermediate. Similarly, the cells that are produced by differentiating stem cells range from multipotent stem or progenitor cells, to terminally differentiated cells, and to all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion and homogeneous differentiation of the cells in a population, and is laborious and of low efficiency.

The provided monolayer differentiation platform facilitates differentiation towards definitive hemogenic endothelium resulting in the derivation of hematopoietic stem cells and differentiated progeny such as T, B, NKT and NK cells. The monolayer differentiation strategy combines enhanced differentiation efficiency with large-scale expansion, and enables the delivery of a therapeutically relevant number of pluripotent stem cell-derived hematopoietic cells for various therapeutic applications. Further, monolayer culturing using the methods provided herein leads to functional hematopoietic lineage cells that enable a full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors. NK cell progenitors, T cells, NK cells, NKT cells, B cells, macrophages, and neutrophils.

The method for directing differentiation of pluripotent stem cells into cells of a definitive hematopoietic lineage, comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs, or naïve iPSCs, or iPSCs comprising one or more genetic imprints; and the one or more genetic imprints comprised in the iPSCs are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73⁻. In some embodiments, the definitive hemogenic endothelium cells are CD34⁺CD43⁻CD93⁻. In some embodiments, the definitive hemogenic endothelium cells are CD34⁺CD93⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34′CD45CD7. In some embodiments of the method, the pluripotent stem cell-derived T cell progenitors are CD45+CD7′.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3-CD45⁺CD56⁺CD7⁺. In some embodiments, the pluripotent stem cell-derived NK cells are CD3⁻CD45⁺CD56⁺, and optionally further defined by being NKp46⁺, CD57⁺ and CD16⁺.

In some embodiments, the genome-engineered iPSC-derived cells obtained from the above methods comprise one or more inducible suicide genes integrated at one or more desired integration sites comprising AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCR$ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, and TIGIT, or other loci meeting the criteria of a genome safe harbor. In some other embodiments, the genome-engineered iPSC-derived cells comprise polynucleotides encoding safety switch proteins, targeting modality, receptors, signaling molecules, transcription factors, pharmaceutically active proteins and peptides, drug target candidates, or proteins promoting trafficking, homing, viability, self-renewal, persistence, and/or survival of stem cells and/or progenitor cells. In some embodiments, the genome-engineered iPSC-derived cells comprising one or more suicide genes further comprise one or more in/dels comprised in one or more endogenous genes associated with immune response regulation and mediation, including, but not limited to, checkpoint genes, endogenous T cell receptor genes, and MHC class I suppressor genes.

Additionally, applicable dedifferentiation methods and compositions for obtaining genomic-engineered hematopoietic cells of a first fate to genomic-engineered hematopoietic cells of a second fate include those depicted in, for example, International Pub. No. WO2011/159726, the disclosure of which is incorporated herein by reference. The method and composition provided therein allows partially reprogramming a starting non-pluripotent cell to a non-pluripotent intermediate cell by limiting the expression of endogenous Nanog gene during reprogramming; and subjecting the non-pluripotent intermediate cell to conditions for differentiating the intermediate cell into a desired cell type.

### V. Therapeutic Use of Derivative Immune Cells with Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed. In some embodiments, the iPSCs comprise one or more targeted genetic edits that are retainable in the iPSC-derived effector cells, wherein the genetically engineered iPSCs and derivative cells thereof are suitable for cell-based adoptive therapies. In one embodiment, the isolated population or subpopulation of genetically engineered effector cells comprises iPSC-derived CD34⁺ cells. In one embodiment, the isolated population or subpopulation of genetically engineered effector cells comprises iPSC-derived HSC cells. In one embodiment, the isolated population or subpopulation of genetically engineered effector cells comprises iPSC-derived proT or T cells. In one embodiment, the isolated population or subpopulation of genetically engineered effector cells comprises iPSC-derived proNK or NK cells. In one embodiment, the isolated population or subpopulation of genetically engineered effector cells comprises iPSC-derived immune regulatory cells or myeloid derived suppressor cells (MDSCs).

In some embodiments, the iPSC-derived genetically engineered effector cells are further modulated ex vivo for improved therapeutic potential. In one embodiment, an isolated population or subpopulation of genetically engineered effector cells that have been derived from iPSCs comprises an increased number or ratio of naïve T cells, stem cell memory T cells, and/or central memory T cells. In one embodiment, the isolated population or subpopulation of genetically engineered effector cells that have been derived from iPSCs comprises an increased number or ratio of type I NKT cells. In another embodiment, the isolated population or subpopulation of genetically engineered effector cells that have been derived from iPSCs comprises an increased number or ratio of adaptive NK cells. In some embodiments, the isolated population or subpopulation of genetically engineered CD34 cells, HSC cells, T cells, NK cells, or myeloid derived suppressor cells derived from iPSCs are allogeneic. In some other embodiments, the isolated population or subpopulation of genetically engineered CD34⁺ cells, HSC cells, T cells, NK cells, or MDSCs derived from iPSC are autologous.

In some embodiments, the iPSC for differentiation comprises genetic imprints selected to convey desirable therapeutic attributes in derived effector cells, provided that cell development biology during differentiation is not disrupted, and provided that the genetic imprints are retained and functional in the differentiated hematopoietic cells derived from said iPSC.

In some embodiments, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retain the source therapeutic attributes, which are also comprised in the iPSC-derived hematopoietic lineage cells.

In some embodiments, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments, the genetically modified iPSC and the derivative cells thereof comprise a genotype listed in Table 1. In some other embodiments, the genetically modified iPSC and the derivative cells thereof comprising a genotype listed in Table 1 further comprise additional genetically modified modalities comprising (1) one or more of deletion or disruption of B2M, TAP1, TAP2, tapasin, NLRC5, CIITA, RFXANK, RFX5, RFXAP, TCRα or TCRβ constant region (TRAC or TRBC), NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3, or TIGIT; and/or (2) introduction of HLA-E, HLA-G, 4-1BBL, CD3, CD4, CD8, CD47, CD113, CD131, CD137, CD80, PDL1, A_{2A}R, CAR, TCR, Fc receptor, or surface triggering receptors for coupling with bi- or multi-specific or universal engagers.

In still some other embodiments, the iPSC-derived hematopoietic lineage cells comprise the therapeutic attributes of the source specific immune cell relating to a combination of at least two of the following: (i) expression of one or more antigen targeting receptors; (ii) modified HLA; (iii) resistance to tumor microenvironment; (iv) recruitment of bystander immune cells and immune modulations; (v) improved on-target specificity with reduced off-tumor effect; (vi) overcoming or reducing tumor microenvironment suppression associated with solid tumors; and (vii) improved homing, persistence, cytotoxicity, or antigen escape rescue.

In some embodiments, the iPSC-derived hematopoietic cells comprise a genotype listed in Table 1, and said cells express a signaling redirector receptor (“SRR”), wherein the signaling redirector receptor comprises a a partial or full peptide of an intracellular domain (ICD) of a cytokine receptor, and a partial or full peptide of an extracellular domain (ECD) of a signaling receptor. In various embodiments, the signaling redirector receptor comprises: (i) an extracellular domain, or a fragment thereof, of transforming growth factor beta receptor (TGFβR), programmed cell death 1 (PD1). CTLA4, IL10R, IL4R, or any combination thereof; and (ii) an intracellular domain, or a fragment thereof, of IL2R, IL12R, IL18R, IL21R, or any combination thereof. In some embodiments, the iPSC-derived hematopoietic cells comprise at least a TGFβ-SRR, and optionally one or more of a cytokine signaling complex, a CAR CD38 knockout, an exogenous CD16 or a variant thereof, and HLA modification; and wherein the iPSC-derived hematopoietic cells optionally comprise at least one cytokine signaling complex comprising a partial or full length of at least one cytokine and/or its receptor thereof, wherein the cytokine comprises IL2, IL4, IL6, IL7, IL9, IL10, IL11. IL12, IL15, IL18, IL21, or any combination thereof. In some embodiments, the cytokine signaling complex and the CAR(s) is NK cell specific. In some other embodiments, the engineered expression of the cytokine signaling complex and the CAR(s) is T cell specific. In some embodiments, the iPSC-derived hematopoietic effector cells are antigen specific. In some embodiments, the antigen specific derivative effector cells target a liquid tumor. In some embodiments, the antigen specific derivative effector cells target a solid tumor. In some embodiments, the antigen specific iPSC-derived hematopoietic effector cells are capable of rescuing tumor antigen escape.

In a further aspect, the present application provides a method of reducing or preventing tumor microenvironment suppression associated with solid tumors and/or allorejection of allogeneic effector cells by recipient activated immune cells in an adoptive cell therapy, wherein the method comprises administering a combination therapy, wherein the combination therapy comprises the derivative effector cells described herein and an anti-CD38 therapeutic agent. In various embodiments, the derivative effector cells comprise a signaling redirector receptor (“SRR”), wherein the SRR comprises a partial or full peptide of an intracellular domain (ICD) of a cytokine receptor, and a partial or full peptide of an extracellular domain (ECD) of a signaling receptor, and optionally further comprise one or more of a cytokine signaling complex, an exogenous CD16, CD38^{-/-}, a CAR, HLA modification, an antibody, a checkpoint inhibitor, an engager, and any other modality, as shown in Table 1. In certain embodiments, the SRR comprises (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R. IL12R, IL18R, IL21R, or any combination thereof. In various embodiments the anti-CD38 therapeutic agent of the combination therapy is an anti-CD38 antibody or fragment thereof. In some embodiments, the anti-CD38 antibody is daratumumab, isatuximab, or MOR202. In some embodiments, the anti-CD38 therapeutic agent is administered with, before, or after the administering of the derivative effector cells. Thus, in some embodiments, the antibody is used in combination with a population of the effector cells described herein by concurrent or consecutive administration to a subject. In other embodiments, such antibody or a fragment thereof may be expressed by the effector cells by genetically engineering an iPSC using an exogenous polynucleotide sequence encoding said antibody or fragment thereof and directing differentiation of the engineered iPSC, as desdribed herein. In some embodiments of the method, the effector cells are iPSC-derived hematopoietic cells. In some embodiments of the method, the effector cells are iPSC-derived T, NK, or NKT cells.

In a further embodiment of the method of reducing or preventing tumor microenvironment suppression associated with solid tumors and/or allorejection of allogeneic effector cells by recipient activated immune cells in an adoptive cell therapy, the method further comprises administering an antibody specific to a same or different upregulated surface protein as targeted by the CAR, and/or one or more additional therapeutic agents. In some embodiments of the method, the antibody comprises at least one of an anti-CD20, an anti-HER2, an anti-CD52, an anti-EGFR, an anti-CD123, an anti-GD2, an anti-PDL1, an anti-CD38 antibody, or any of the humanized or Fc modified variants or fragments, functional equivalents and biosimilars thereof. In some embodiments of the therapeutic agents used in the method, the therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

A variety of diseases may be ameliorated by introducing the derivative effector cells of the invention to a subject suitable for adoptive cell therapy. In some embodiments, the iPSC-derived hematopoietic cells as provided herein are for allogeneic adoptive cell therapies. In other embodiments, the iPSC-derived hematopoietic cells as provided herein are for preventing or reducing tumor microenvironment suppression associated with solid tumors. Additionally, the present invention provides, in some embodiments, therapeutic use of the above therapeutic compositions and/or combination therapies by introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes.

Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus.

Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease. Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's).

Examples of viral infections include, but are not limited to, HIV—(human immunodeficiency virus), HSV—(herpes simplex virus), KSHV—(Kaposi's sarcoma-associated herpesvirus), RSV—(Respiratory Syncytial Virus), EBV—(Epstein-Barr virus), CMV—(cytomegalovirus), VZV (Varicella zoster virus), adenovirus-, a lentivirus-, a BK polyomavirus-associated disorders.

The treatment using the derived hematopoietic lineage cells of embodiments disclosed herein could be carried out upon symptom presentation, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent or composition may be administered before, during or after the onset of a disease or an injury. Treatment of ongoing disease, where the treatment stabilizes or reduces the undesirable clinical symptoms of the patient, is also of particular interest.

In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the derived hematopoietic lineage cells of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

The therapeutic composition comprising iPSC-derived hematopoietic lineage cells as disclosed herein can be administered to a subject before, during, and/or after other treatments. As such a method of a combinational therapy can involve the administration or preparation of iPSC-derived effector cells before, during, and/or after the use of an additional therapeutic agent. As provided above, the one or more additional therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, a non-drug therapy, such as, surgery.

In some embodiments of a combinational cell therapy, the therapeutic combination comprises the iPSC-derived hematopoietic lineage cells provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered iPSC-derived hematopoietic lineage cells include, but are not limited to, anti-CD20 antibodies (e.g., rituximab, veltuzumab, ofatumumab, ublituximab, ocaratuzumab, obinutuzumab), anti-HER2 antibodies (e.g., trastuzumab, pertuzumab), anti-CD52 antibodies (e.g., alemtuzumab), anti-EGFR antibodies (e.g., cetuximab), anti-GD2 antibodies (e.g., dinutuximab), anti-PDL1 antibodies (e.g., avelumab), anti-CD38 antibodies (e.g., daratumumab, isatuximab, MOR202), anti-CD123 antibodies (e.g., 7G3, CSL362), anti-SLAMF7 antibodies (elotuzumab), and their humanized or Fc modified variants or fragments or their functional equivalents or biosimilars.

In some embodiments, the additional therapeutic agent comprises one or more checkpoint inhibitors. Checkpoints are referred to cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells, including NK or T cells, are provided above.

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK cells as provided. In some embodiments, the effector cells for combination therapy as described herein are derivative T cells. In some embodiments, the derivative NK or T cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, avelumab, durvalumab, ipilimumab, IPH4102, IPH43, IPH33, lirilumab, monalizumab, nivolumab, pembrolizumab, and their derivatives or functional equivalents.

The combination therapies comprising the derivative effector cells and one or more check inhibitors are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et Varioliformis acuta, CD30⁺ cutaneous T-cell lymphoma, Secondary cutaneous CD30⁺ large cell lymphoma, non-mycosis fungoides CD30 cutaneous large T-cell lymphoma, Pleomorphic T-cell lymphoma, Lennert lymphoma, subcutaneous T-cell lymphoma, angiocentric lymphoma, blastic NK-cell lymphoma, B-cell Lymphomas, hodgkins lymphoma (HL), Head and neck tumor: Squamous cell carcinoma, rhabdomyocarcoma. Lewis lung carcinoma (LLC), non-small cell lung cancer, esophageal squamous cell carcinoma, esophageal adenocarcinoma, renal cell carcinoma (RCC), colorectal cancer (CRC), acute myeloid leukemia (AML), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma.

In some embodiments, other than the derivative effector cells as provided herein, a combination for therapeutic use comprises one or more additional therapeutic agents comprising a chemotherapeutic agent or a radioactive moiety. Chemotherapeutic agent refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, examples of which are known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and granicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethistilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxvurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistframe.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

Other than an isolated population of iPSC-derived hematopoietic lineage cells included in the therapeutic compositions, the compositions suitable for administration to a patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of embodiments of the present invention (see, e.g., Remington's Pharmaceutical Sciences, 17^{th} ed. 1985, the disclosure of which is hereby incorporated by reference in its entirety).

In one embodiment, the therapeutic composition comprises the pluripotent cell derived T cells made by the methods and composition disclosed herein. In one embodiment, the therapeutic composition comprises the pluripotent cell derived NK cells made by the methods and composition disclosed herein. In one embodiment, the therapeutic composition comprises the pluripotent cell derived CD34⁺ HE cells made by the methods and composition disclosed herein. In one embodiment, the therapeutic composition comprises the pluripotent cell derived HSCs made by the methods and composition disclosed herein. In one embodiment, the therapeutic composition comprises the pluripotent cell derived MDSC made by the methods and composition disclosed herein. A therapeutic composition comprising a population of iPSC-derived hematopoietic lineage cells as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, a buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of embodiments of the present invention. Such compositions are suitable for administration to human subjects. Generally speaking, any medium that supports the maintenance, growth, and/or health of the iPSC-derived effector cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in the present invention and that there are many suitable media known and available to those in the art.

The iPSC-derived hematopoietic lineage cells can have at least 50%, 60%, 70%, 80%, 90%, 95%, 98%, or 99% T cells, NK cells, NKT cells, proT cells, proNK cells, CD34⁺ HE cells, HSCs, B cells, myeloid-derived suppressor cells (MDSCs), regulatory macrophages, regulatory dendritic cells, or mesenchymal stromal cells. In some embodiments, the iPSC-derived hematopoietic lineage cells have about 95% to about 100% T cells, NK cells, proT cells, proNK cells, CD34⁺ HE cells, or myeloid-derived suppressor cells (MDSCs). In some embodiments, the present invention provides therapeutic compositions having purified T cells or NK cells, such as a composition having an isolated population of about 95% T cells, NK cells, proT cells, proNK cells, CD34⁺ HE cells, or myeloid-derived suppressor cells (MDSCs) to treat a subject in need of the cell therapy.

One aspect of the present application provides a method of treating a subject in need by administering one or more therapeutic doses of effector cells comprising at least a signaling redirector receptor (SSR), wherein the SSR comprises a partial or full peptide of an extracellular domain (ECD) of a signaling receptor, and a partial or full peptide of an intracellular domain (ICD) of a cytokine signaling receptor, wherein the signaling receptor and the cytokine receptor are different molecules. In various embodiments, the signaling redirector receptor comprises (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R, IL12R, IL18R, IL21R, or any combination thereof.

Another aspect of the invention provides a method of sensitizing tumor cells, wherein the method comprises contacting the tumor cells with a recombinant receptor comprising a partial or full peptide of an extracellular domain (ECD) of a signaling receptor, and a partial or full peptide of an intracellular domain (ICD) of a cytokine signaling receptor, wherein the signaling receptor and the cytokine receptor are different molecules, thereby inhibiting or reducing the signaling of TGFβ expressed by, or in the environment of, the tumor cells. In various embodiments, the signaling redirector receptor comprises (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R, IL12R, IL18R, IL21R, or any combination thereof. In some embodiments, the recombinant receptor is expressed by immune effector cells engineered with a polynucleotide encoding the recombinant receptor as provided herein. In some embodiments, the immune effector cells are administered to a subject under a therapy in need thereof. In some embodiments, the immune effector cells are derived from iPSCs comprising a polynucleotide encoding the recombinant receptor. In some embodiments, the immune effector cells are NK cells, T cells, or a combination thereof. In some embodiments, the immune effector cells are autologous or allogeneic to the subject in need of tumor sensitizing. In particular embodiments, the tumor cells are of a solid tumor as provided herein. In various embodiments, the effector cells may be provided prior to or concurrently with one or more additional therapeutic agents, as described above.

Another aspect of the present application provides a method of treating a subject in need using a combinational cell therapy. In some embodiments of the combinational cell therapy, the method of treating a subject in need comprises administering one or more therapeutic doses of effector cells comprising at least a signaling redirector receptor comprising a partial or full peptide of an extracellular domain (ECD) of a signaling receptor, and a partial or full peptide of an intracellular domain (ICD) of a cytokine receptor, wherein the signaling receptor and the cytokine receptor are from different molecules, and wherein the effector cells optionally further comprise one or more of a CAR, an exogenous CD16 or a variant thereof. CD38 knockout, an exogenous cytokine signaling complex and HLA deficiency. In some embodiments, the effector cells are provided as part of a combinational cell therapy wherein the cells further comprise one or more therapeutic agents comprising a peptide, a cytokine, a checkpoint inhibitor, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In various embodiments, the combinational cell therapy, or composition used therefor, comprises a population of effector cells derived from genomically engineered iPSCs and one or more therapeutic agents, wherein the engineered iPSCs and the derived effector cells comprise a signaling redirector receptor comprising (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R, IL12R, IL18R, IL21R, or any combination thereof, wherein said cells further optionally comprise one or more of a CAR, exogenous CD16 or a variant thereof, CD38 knockout, an exogenous cytokine signaling complex, or other edits as described herein, or a genotype listed in Table 1.

As a person of ordinary skill in the art would understand, both autologous and allogeneic hematopoietic lineage cells derived from iPSC based on the methods and composition herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells or T cells comprising a signaling redirector receptor, wherein the signaling redirector receptor comprises a partial or full peptide of an extracellular domain (ECD) of a signaling receptor, and a partial or full peptide of an intracellular domain (ICD) of a cytokine receptor, wherein the signaling receptor and the cytokine receptor are different molecules and wherein the NK cells or T cells optionally comprise one or more modalities listed in Table 1. In various embodiments, the signaling redirector receptor comprises (i) an extracellular domain, or a fragment thereof, of transforming growth factor beta receptor (TGFβR), programmed cell death 1 (PD1), CTLA4, IL10R, IL4R, or any combination thereof; and (ii) an intracellular domain, or a fragment thereof, of IL2R, IL12R, IL18R, IL21R. or any combination thereof.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose: about 3×10⁹ cells to about 3×10¹⁰ cells, per dose, or any range in-between. Generally, 1×10⁸ cells/dose translates to 1.67×10⁶ cells/kg for a 60 kg patient/subject.

In one embodiment, the number of derived hematopoietic lineage cells in the therapeutic composition is the number of immune cells in a partial or single cord of blood, or is at least 0.1×10⁵ cells/kg of body weight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of body weight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between.

The compositions comprising a population of derived hematopoietic lineage cells of the invention can be sterile, and can be suitable and ready for administration (i.e., can be administered without any further processing) to human patients/subjects. A cell-based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents including small chemical molecules. The compositions and methods for modulating immune cells including iPSC-derived effector cells are described in greater detail, for example, in International Pub. No. WO2017/127755, the relevant disclosure of which is incorporated herein by reference. For derived hematopoietic lineage cells that are genetically engineered to express recombinant TCR or CAR, the cells can be activated and expanded using methods as described, for example, in U.S. Pat. No. 6,352,694.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810, the disclosures of which are incorporated by reference, for artificial antigen presenting cells (aAPCs) that are contemplated for use in activating and expanding T lymphocytes in embodiments of the present invention.

Some variation in dosage, frequency, and protocol will necessarily occur depending on the condition of the subject being treated. The person responsible for administration will, in any event, determine the appropriate dose, frequency and protocol for the individual subject.

### EXAMPLES

The following examples are offered by way of illustration and not by way of limitation.

### Example 1—Materials and Methods

To effectively select and test suicide systems under the control of various promoters in combination with different safe harbor loci integration strategies, a proprietary hiPSC platform of the applicant was used, which enables single cell passaging and high-throughput, 96-well plate-based flow cytometry sorting, to allow for the derivation of clonal hiPSCs with single or multiple genetic modulations.

hiPSC Maintenance in Small Molecule Culture: hiPSCs were routinely passaged as single cells once confluency of the culture reached 75%-90%. For single-cell dissociation, hiPSCs were washed with PBS (Mediatech) and treated with Accutase (Millipore) for 3-5 min at 37° C. The single-cell suspension was then mixed with conventional medium, centrifuged, resuspended in FMM, and plated on Matrigel-coated surface. Passages were typically 1:6-1:8, transferred tissue culture plates previously coated with Matrigel and fed every 2-3 days with FMM. Cell cultures were maintained in a humidified incubator set at 37° C. and 5-10% CO₂.

Human IPSC engineering with ZFN, CRISPR for targeted editing of modalities of interest: Using ROSA26 targeted insertion as an example, for ZFN mediated genome editing, 2 million iPSCs were transfected with a mixture of 2.5 μg ZFN-L, 2.5 μg ZFN-R and 5 μg donor construct, for AAVS1 targeted insertion. For CRISPR mediated genome editing, 2 million iPSCs were transfected with a mixture of 5 μg ROSA26-gRNA/Cas9 and 5 μg donor construct, for ROSA26 targeted insertion. Transfection was done using Neon transfection system (Life Technologies). On day 2 or 3 after transfection, transfection efficiency was measured using flow cytometry if the plasmids contain artificial promoter-driven GFP and/or RFP expression cassette.

Bulk sort and clonal sort of genome-edited IPSCs: iPSCs with genomic targeted editing using ZFN or CRISPR-Cas9 were bulk sorted and clonal sorted of GFP⁺SSEA4⁺TRA181⁺ iPSCs. Single cell dissociated targeted iPSC pools were resuspended in staining buffer containing Hanks' Balanced Salt Solution (MediaTech), 4% fetal bovine serum (Invitrogen), 1× penicillin/streptomycin (Mediatech) and 10 mM Hepes (Mediatech), made fresh for optimal performance. Conjugated primary antibodies, including SSEA4-PE, TRA181-Alexa Fluor-647 (BD Biosciences), were added to the cell solution. The solution was washed in staining buffer, spun and resuspended in staining buffer containing 10 μM Thiazovivn for flow cytometry sorting. Flow cytometry sorting was performed on FACS Aria II (BD Biosciences). Upon completion of the sort, the 96-well plates incubated. Colony formation was detected as early as day 2 and most colonies were expanded between days 7-10 post sort. In the first passage, wells were washed with PBS and dissociated with 30 μL Accutase The dissociated colony is transferred to another well of a 96-well plate previously coated with 5× Matrigel. Subsequent passages were done routinely. Each clonal cell line was analyzed for GFP fluorescence level and TRA1-81 expression level. Clonal lines with near 100% GFP⁺ and TRA1-81⁺ were selected for further PCR screening and analysis including but not limited to off-target editing, and/or karyotype of the engineered iPSCs, before the clonal population is cryopreserved to serve as a master cell bank. Flow cytometry analysis was performed on Guava EasyCyte 8 HT (Millipore) and analyzed using Flowjo (FlowJo, LLC).

Flow Cytometry of Effectors: For CAR-iT and primary T cell experiments, cells were stained with a fixable viability dye (BD Biosciences, San Jose, CA) and then washed with PBS. For CAR detection, the cells were incubated with Alexa Flour 647 conjugated goat anti-mouse IgG F(ab)2 (Jackson ImmunoResearch Laboratories, West Grove PA). Cells were subsequently washed and stained with Pe-Cy7 conjugated or PE-conjugated antibodies (Biolegend, San Diego, CA). For intracellular staining, cells were fixed with BD Phosflow Fix Buffer (BD Biosciences, San Jose, CA), and permeabilization with BD Phosflow Perm Buffer (BD Biosciences, San Jose, CA). Following permeabilization, samples were incubated with PE-CF594-conjugated anti-STAT5 (pY694) (BD Biosciences, San Jose, CA) and BV421-conjugated anti-pSTAT3 (S727) (BD Biosciences, San Jose, CA). After washing, all samples were resuspended in PBS with 0.2% human serum albumin containing counting beads. All sample data were acquired using LSRFortessa™ X-20 (BD Biosciences, San Jose, CA) and analyzed using Flowjo™ software (Ashland, OR).

For CAR-iNK cells, cells were stained with a fixable viability dye (BD Biosciences, San Jose, CA). After washing, cells were incubated with 10 μg/ml of recombinant human NKG2D blocking antibody (Biolegend, San Diego, CA) and 0.5 mg/ml of recombinant FC Avidin (R&D systems, Minneapolis, MN) on ice. After washing, cells were incubated with PE-conjugated streptavidin followed by biotinylated anti-strepavidin (Vector laboratories, Burlingame, CA). CAR frequency and counts were measured by flow cytometry using counting beads. All sample data were acquired using LSRFortessam X-20 (BD Biosciences, San Jose, CA) and analyzed using Flowjo™ software (Ashland, OR).

### Example 2—Tumor Growth Inhibition is Enhanced in the Presence of Cytokines

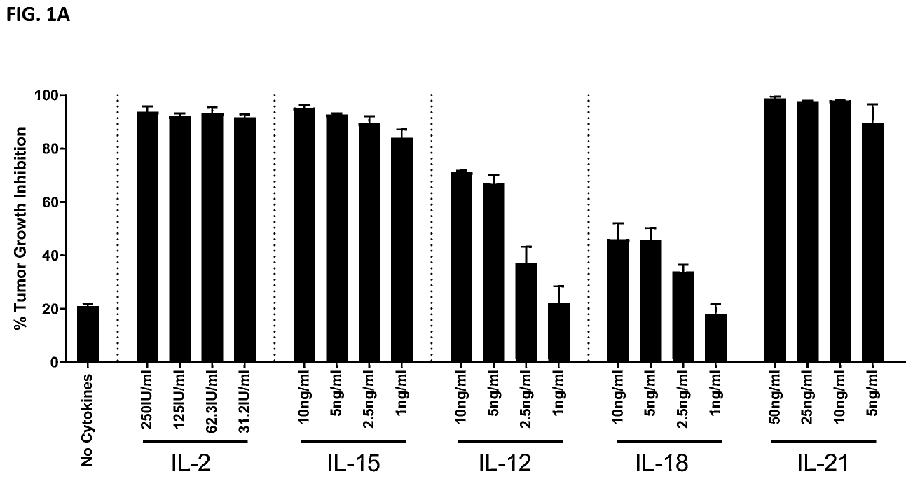

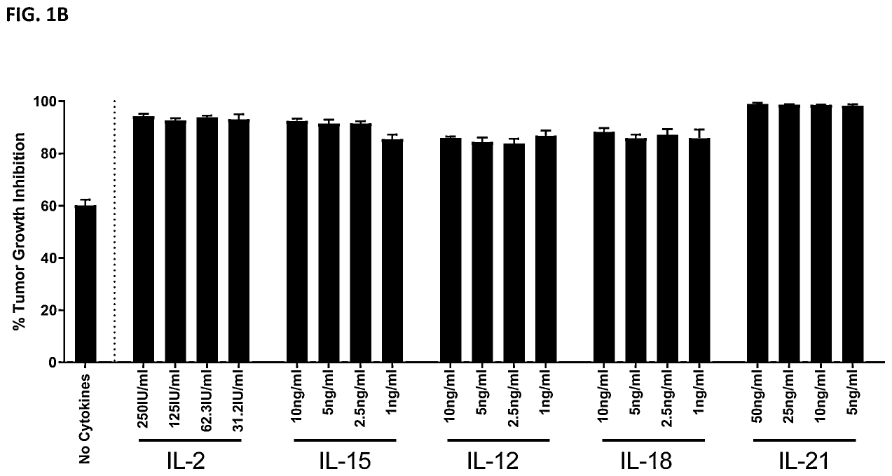

TGFβ is an immunosuppressive cytokine present in the tumor microenvironment that remains a considerable challenge in the treatment of solid tumors by cellular therapy. To test if selected cytokines could enhance CAR-iT cell anti-tumor function, fully differentiated CAR19-iT cells were used for proof-of-concept and co-cultured with Nalm6 tumor cells at a suboptimal effector to target ratio of 1:4 in round bottom 96-well plates. Recombinant IL2, or IL15, or IL18, or IL21 (R&D systems, Minneapolis, MN) was added to the co-culture medium at different concentrations. The co-cultures were analyzed by flow cytometry to measure tumor cell numbers on day 7 of the assay. Tumor growth inhibition was calculated by normalizing to a culture with tumor cells only. As shown in FIG. 1A, tumor growth inhibition (TGI) by the CAR19-iT cells was measured at about 20% with no cytokine in the co-culture media (control), while addition of cytokine to the co-cultures resulted in >85% tumor growth inhibition even at the lowest cytokine concentration tested. It was observed that treatment with IL12 and IL18 enhanced tumor growth inhibition in a cytokine concentration-dependent manner.

In a separate experiment, CAR19-iT cells were engineered to express an IL7 Receptor fusion transgene (IL7RF) and subjected to the same cytotoxicity assay as described above. As shown in FIG. 1B, the CAR19-iT cells expressing IL7RF without cytokine co-culture showed ˜60% tumor growth inhibition after 7 days, whereas addition of cytokine further enhances TGI, with all cytokine concentrations tested resulting in >85% tumor growth inhibition across the concentration level of all tested cytokines.

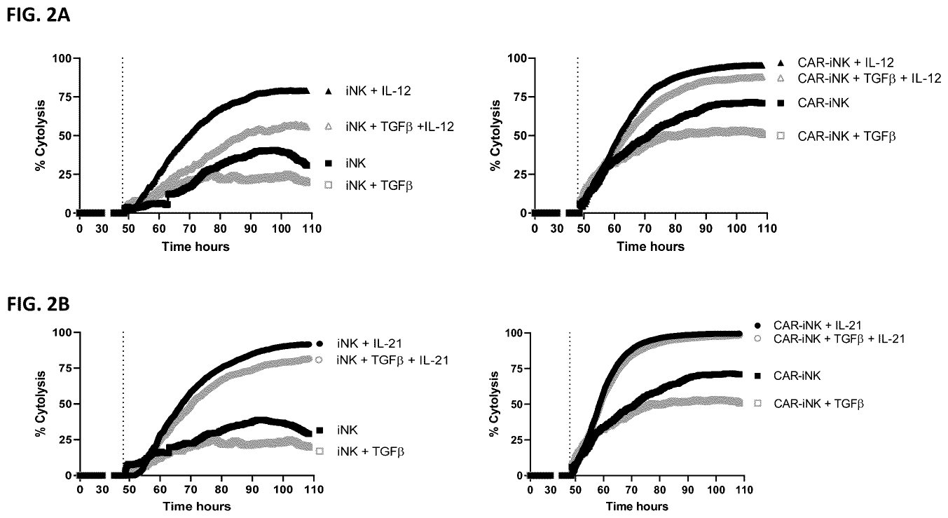

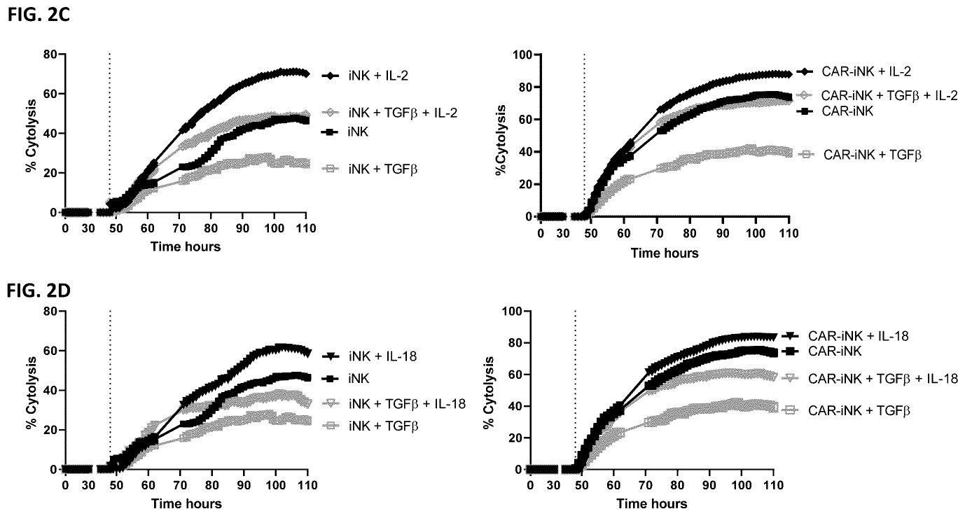

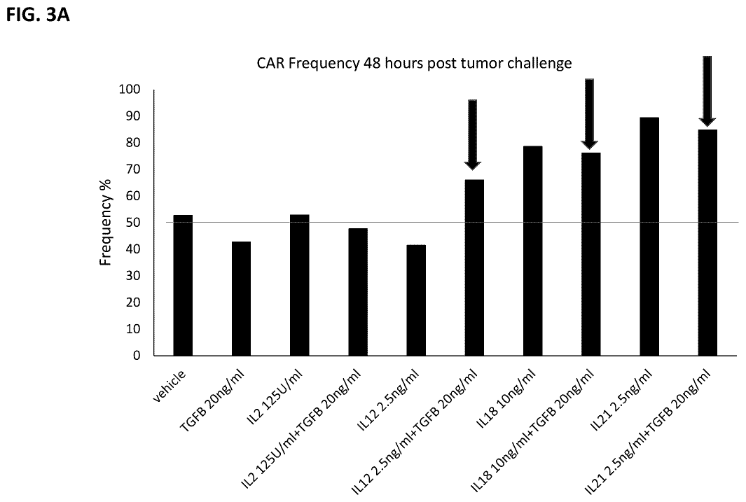

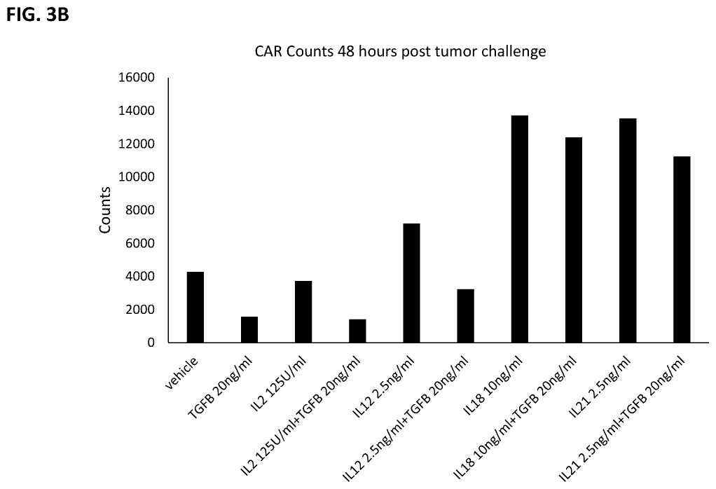

To test if selected cytokines could prevent TGFβ-mediated suppression of iNK cells in vitro, fully differentiated iNK cells (FIGS. 2A-2D, left panels) and CAR-iNK cells (FIGS. 2A-2D, right panels) were co-cultured with cervical tumor cells in media containing either IL12 (FIG. 2A), IL21 (FIG. 2B), IL2 (FIG. 2C), or IL18 (FIG. 2D) in the presence or absence of TGFβ. After measuring background readings, 7e3 CaSkI tumor cells were seeded on RTCA E-Plates (Agilent, Santa Clara, CA). Once a cell index of 1 was reached, media was removed from each well and effectors were added at a 3 to 1 effector to target ratio in the presence of cytokine IL2, IL12, IL18, or IL21 with or without TGFβ and the cell index of each well was measured every 15 minutes on the xCELLigence RTCA system Plates (Agilent, Santa Clara CA) for 48 hours. After 48 hours of co-culture, TGFβ treated iNK and CAR-iNK cells exhibited reduced cytotoxicity. This effect was mitigated, however, when IL2, IL12, IL18 or IL21 were added to the co-culture (FIGS. 2A-2D). Addition of IL12, IL18, or IL21 to the co-culture also enhanced the frequency (FIG. 3A) and number (FIG. 3B) of effector cells when compared to vehicle controls.

### Example 3—TGFβ Redirection Activates Effector Cell Signaling and Improves Effector Cell Functionality

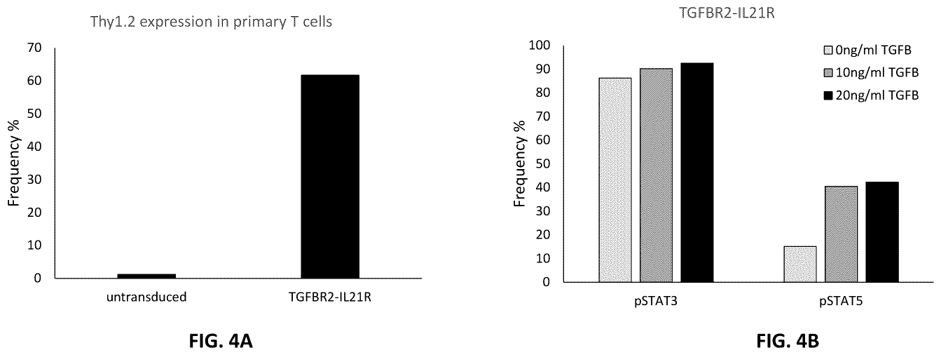

It was then investigated whether it is possible that a chimeric TGFβ receptor composed of a TGFβR2 ectodomain fused to select cytokine intracellular domains could transform TGFβ's immunosuppressive signal into an activating signal in CAR-iT and CAR-iNK cells. To assess whether downstream pathways can be selectively activated via TGFβ signal redirection, primary T cells were transduced with a TGFβ signal redirection receptor (TGFβ-SRR, or also referred to as “TGFβ redirector”) comprising an intracellular domain of IL21R43 (TGFβR2-IL21Rβ). Transduction efficiency was assessed after 6 days and 62% of the T cells expressed the transgene (FIG. 4A). Addition of recombinant TGFβ in the co-culture medium showed a TGFβ-dependent increase in pSTAT3 and pSTAT5 positive T cells as measured by intracellular staining and flow cytometric analysis (FIG. 4B).

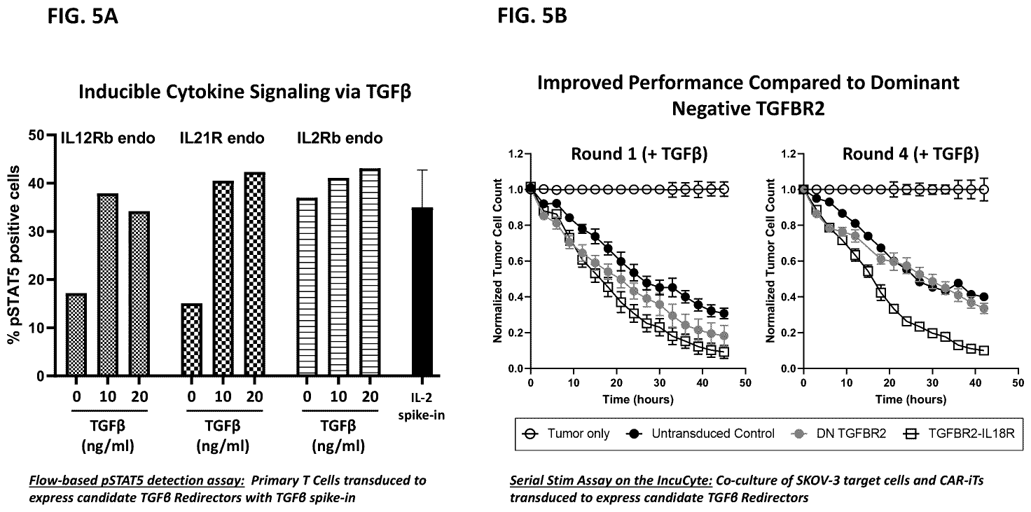

In a separate experiment, groups of activated primary CD8 T cells were each lentivirally transduced with a different TGFβ-SRR construct comprising an extracellular domain from TGFβR and an intracellular domain, or a fragment thereof, of IL2R, IL12R, IL18R. or IL21R. Following a 24-hour rest in T cell media without IL2, the T cells were exposed to the indicated concentrations of TGFβ. After 2 hours, the cells were harvested and analyzed by flow cytometry for the presence of phosphorylated STAT5. IL2 spike-in was used as a positive control. As shown in FIG. 5A, the flow data demonstrates functionality of the cytokine receptor endodomains of IL12R, IL21R and IL2R in the TGFβ-SRR constructs in the presence of TGFβ, showing that the percent of pSTAT5 positive CD8 T cells increased to a level similar to what was observed when IL2 was spiked-in.

In a further experiment, iPSC-derived CAR-T cells (CAR-iTs) were lentivirally transduced to express either dominant negative TGFβR2 (DN TGFβR2 or dnTGFβR2) or a TGFβ-SRR construct (e.g., TGFβR2-IL18R). The CAR-iTs were then tested in a serial stimulation assay, where the ability of effector cells in killing tumor cells was measured on an IncuCyte instrument over multiple rounds of co-culture and in the presence of 20 ng/mL TGFβ. For the TGFβ-SRR, the data related to the TGFβR2-IL18R is shown. As shown in FIG. 5B, CAR-iTs expressing TGFβR2-IL18R showed enhanced tumor killing in the presence of TGFβ in the first round of co-culture (FIG. 5B, left plot) compared to TGFβ-SSR⁻ CAR-iTs or dnTGFβR2⁺ CAR-iTs. After four rounds of co-culture and exposure to TGFβ (FIG. 5B, right plot), the dnTGFβR2⁺ CAR-iTs showed very similar tumor killing kinetics as in the co-culture with untransduced effectors. Importantly, TGFβR2-IL18R CAR-iTs continued to show enhanced tumor killing ability because of the additional cytokine signaling provided by the TGFβ redirector, in this case, through an intracellular domain of IL18R.

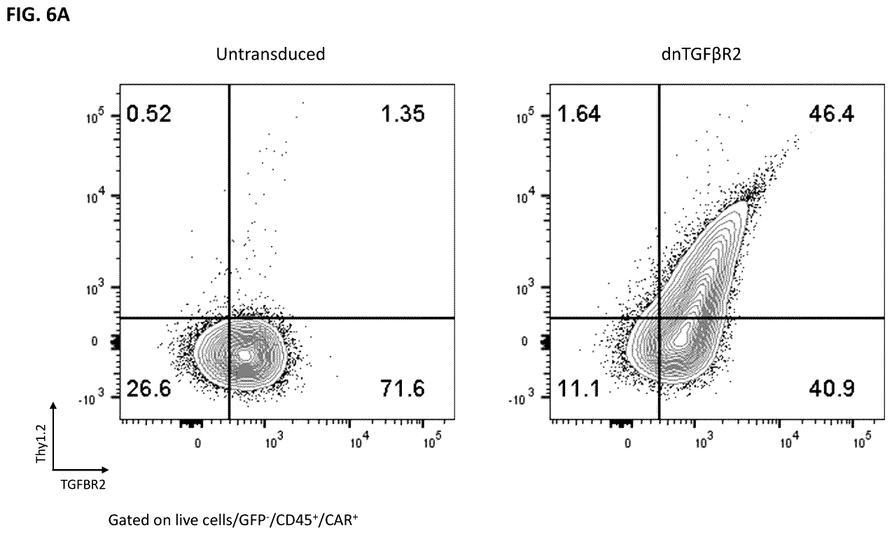

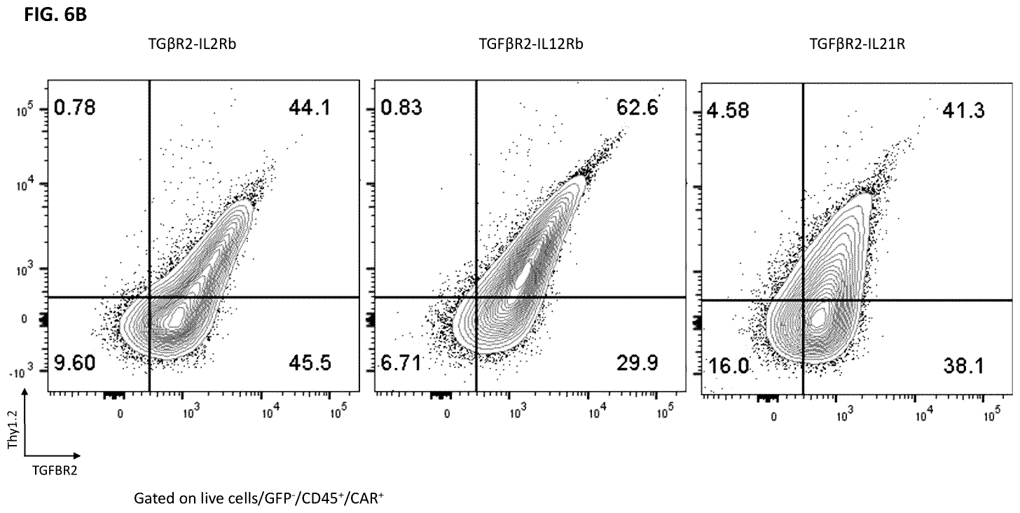

Further, fully differentiated CAR19-iT cells were transduced with lentiviral particles encoding dnTGFβR2, TGFβR2-IL2Rβ, TGFβR2-IL12R4β, TGFβR2-IL18Rβ, or TGFβR2-IL21Rβ at a multiplicity of infection (MOI) of 5 and were tested to determine whether a TGFβ redirection approach can allow CAR19-iT cells to resist the immunosuppressive effect of TGFβ. Expression of the TGFβ-SSRs in iT cells was confirmed by flow cytometry (FIGS. 6A-6B). Following transduction, cells were rested overnight and then co-cultured with Nalm6-GFP⁺ cells at an effector to target ratio of either 1:1 or 2:1, in the presence or absence of recombinant TGFβ (R&D systems, Minneapolis, MN). Target cells and fresh media containing vehicle or recombinant TGFβ was added to the culture every other day. Tumor cell and CAR counts were measured every 48 hours as a readout. Thy1.2 and TGFβR2 were measured throughout the assay by flow cytometry.

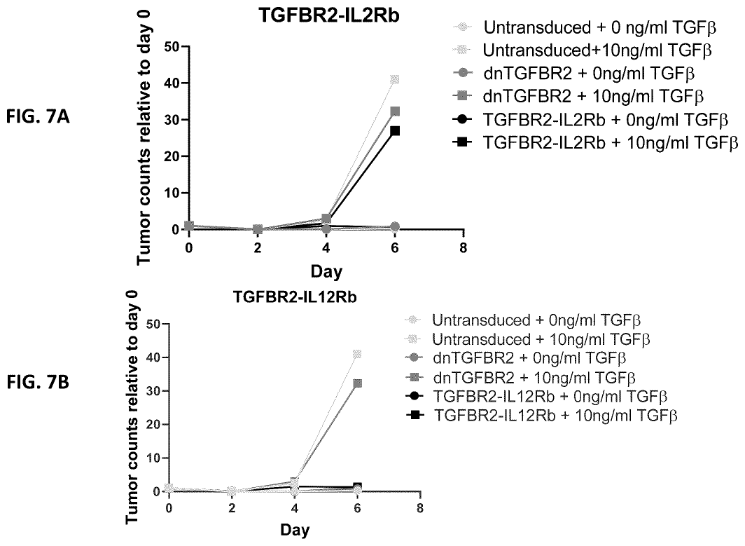

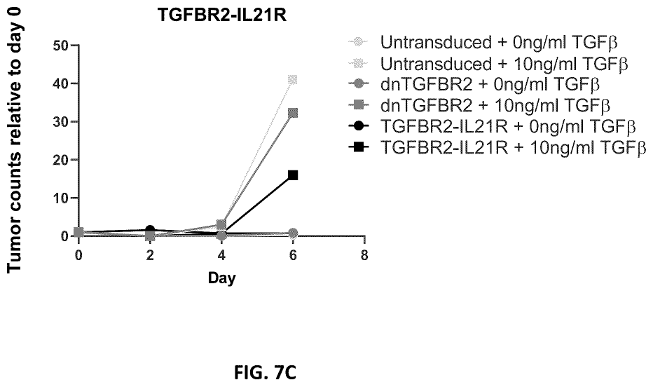

Serial stimulation of transduced CAR19-iT cells with tumor cells lines expressing the cognate antigen showed that after around 6 days of stimulation, iT cells expressing TGFβR2-IL2Rβ (FIG. 7A), TGFβR2-IL12Rβ (FIG. 7B), or TGFβR2-IL21Rβ (FIG. 7C) showed enhanced tumor growth control in the presence of recombinant TGFβ in comparison to CAR19-iT cells expressing dnTGFβR2. After three rounds of restimulation, the increase in tumor cell numbers for non-engineered and dnTGFβR2 CAR-iT cells were measured, showing 41-fold and 32-fold increase over base input, respectively. In contrast, the TGFβ redirection constructs improved the ability of CAR-iT cells to control tumor cell growth with great efficiency, limiting tumor growth to 1.5-fold over three rounds of restimulation. It is therefore indicated that the TGFβ-SSRs as disclosed herein had activated beneficial signaling pathways downstream of their intracellular domains and had contributed to improved effector function of the cells.

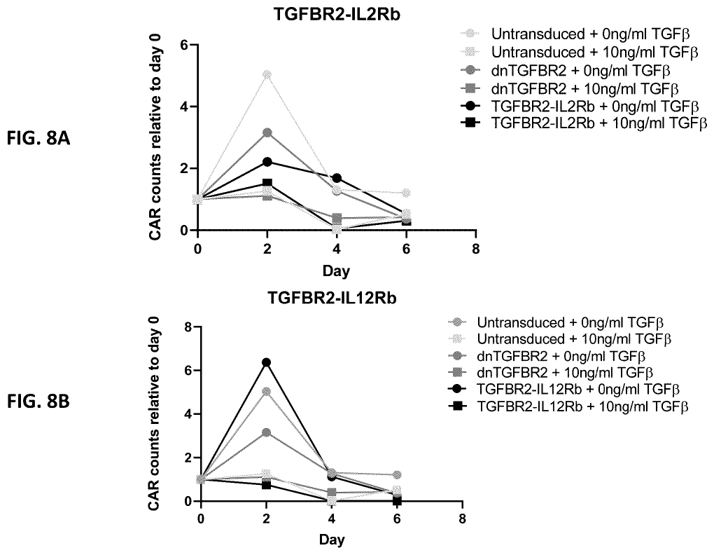

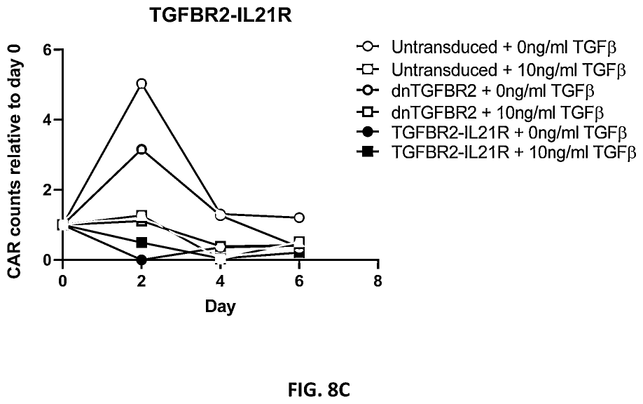

CAR19-iT cell numbers were determined by flow cytometry to determine whether co-cultures with effectors expressing TGFβR2-IL2Rb (FIG. 8A), TGFβR2-IL12Rβ (FIG. 8B), or TGFβR2-IL21R (FIG. 8C) showed any differences in comparison to untransduced or dnTGFβR2 controls in the presence or absence of TGFβ. Since expression of modified TGFβ receptors on iT cells did not alter effector cell number, the mechanism underlying the increased anti-tumor activity, without being limited by theory, may indeed rely on enhanced effector function.

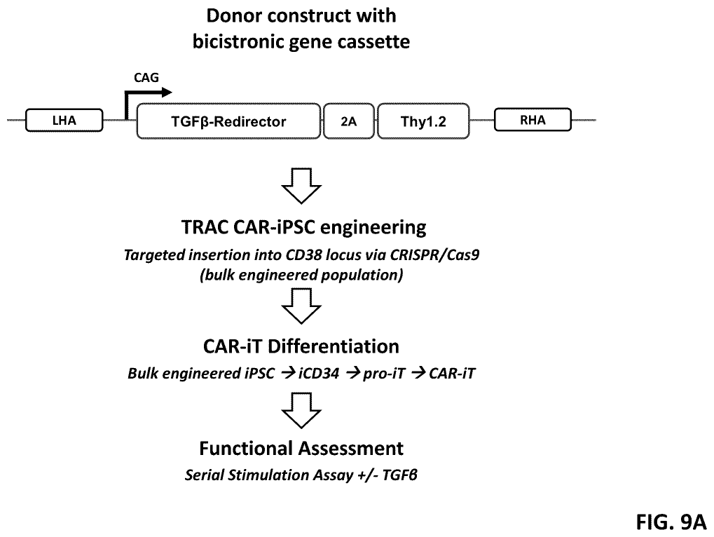

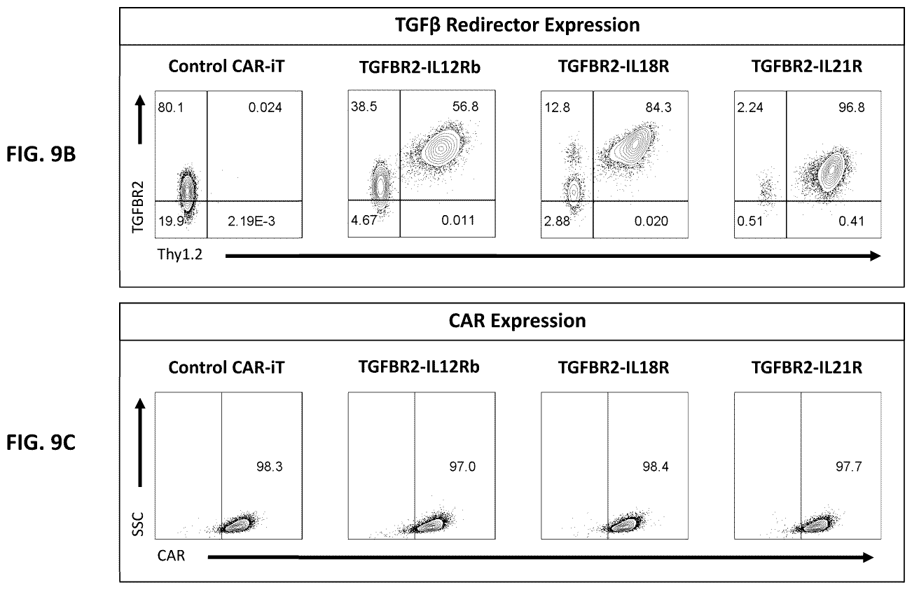

In a further experiment, a population of CRISPR-engineered CAR-iTs that express TGFβ-SSRs was prepared using the strategy shown in FIG. 9A. In particular, a bi-cistronic donor cassette was inserted in the CD38 locus of an iPSC population via CRISPR/Cas9, where the iPSC has a CAR inserted at the TRAC locus. The bulk-engineered iPSCs were differentiated into CAR-iT cells and tested in a serial stimulation assay in the presence or absence of TGFβ. Co-expression of the Thy1.2 marker and the TGFβ-SRR indicates the percentage of cells in the CAR-iT population that were successfully engineered via CRISPR/Cas9. As shown in FIG. 9B, three examples of TGFβ-SSRs that were expressed in CAR-iTs were generated—TGFβR2-IL12Rβ, TGFβR2-IL18R, and TGFβR2-IL21R. Towards the end of the iT differentiation process, when the cells have committed to the T cell lineage, the TRAC promoter becomes active. As shown in FIG. 9C, surface expression of the CAR in the TGFβ-SRR engineered cells indicate successful differentiation into T cells.

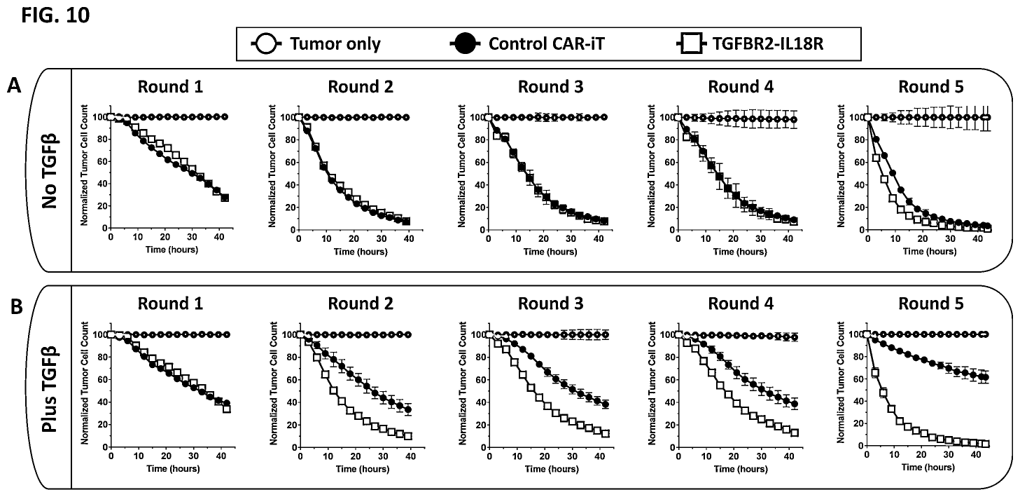

Using a serial stimulation assay, the CRISPR-engineered CAR-iT cells expressing the TGFβR2-IL18R construct were tested for their ability to resist TGFβ-mediated suppression of effector function. In the absence of TGFβ, the control CAR-iTs showed very similar tumor killing kinetics as the TGFβR2-IL18R CAR-iTs over five rounds of co-culture (part A of FIG. 10, No TGFβ), which demonstrates that TGFβR2-IL18R CAR-iTs would not exhibit unnecessarily high levels of effector function in the absence of TGFβ. With the addition of 20 ng/mL TGFβ to the serial stimulation assay, the control CAR-iT cells exhibited similar cytolytic capacity as the TGFβR2-IL18R CAR-iTs in the first round, demonstrating that acute exposure of TGFβ-SRR CAR-iTs with the TGFβ is not likely to dramatically change the effector function of these cells (part B of FIG. 10, Plus TGFβ). However, an increase in tumor killing activity in the TGFβR2-IL18R CAR-iTs was observed starting in the second round of serial stimulation. As shown in part B of FIG. 10, this increase in effector function persisted for up to five rounds of co-culture. In comparison, the control CAR-iTs progressively lost their ability to kill target cells in the serial stimulation assay with TGFβ spike-in.

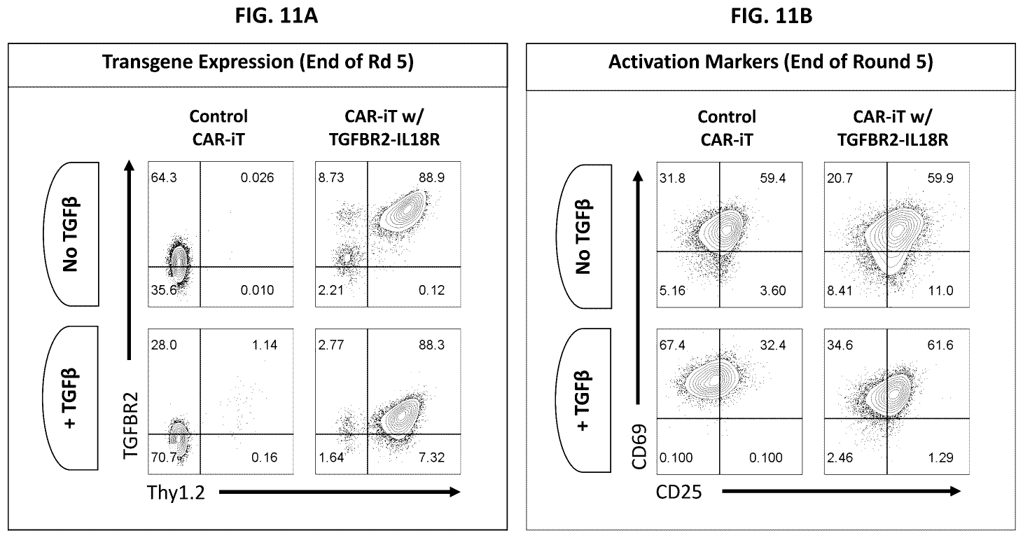

Effector function and antitumor activity of T cells are linked to T cell activation state. To characterize the activation profiles of CAR-iT cells with the TGFβ-SRR after multiple rounds of stimulation with target cells, control CAR-iTs or TGFβ-SRR CAR-iTs were subjected to five rounds of co-culture with target cells in the presence or absence of TGFβ, and at the end of round 5, the cells were stained for TGFβR2 and Thy1.2 (FIG. 11A). Co-expression of the Thy 1.2 marker and TGFβR2 indicates the percent of effector cells that maintained expression of the TGFβ-SRR. The effector cells at the end of five rounds of co-culture with and without TGFβ were also analyzed by flow cytometry for co-expression of the activation markers CD69 and CD25 (FIG. 11B). As shown in FIG. 11B, in the absence of TGFβ, both control and TGFβR2-IL18R CAR-iTs were ˜60% positive for both CD69 and CD25. In comparison, in the presence of TGFβ, the control CAR-iTs showed a reduction in the CD69⁺CD25⁺ cells (32.4%), while 61.6% of the TGFβR2-IL18R CAR-iTs were double positive for CD69 and CD25 (FIG. 11B). Therefore, CAR-iT cells with the TGFβ-SRR as disclosed herein possess an enhanced activation profile even after multiple rounds of stimulation with target cells.

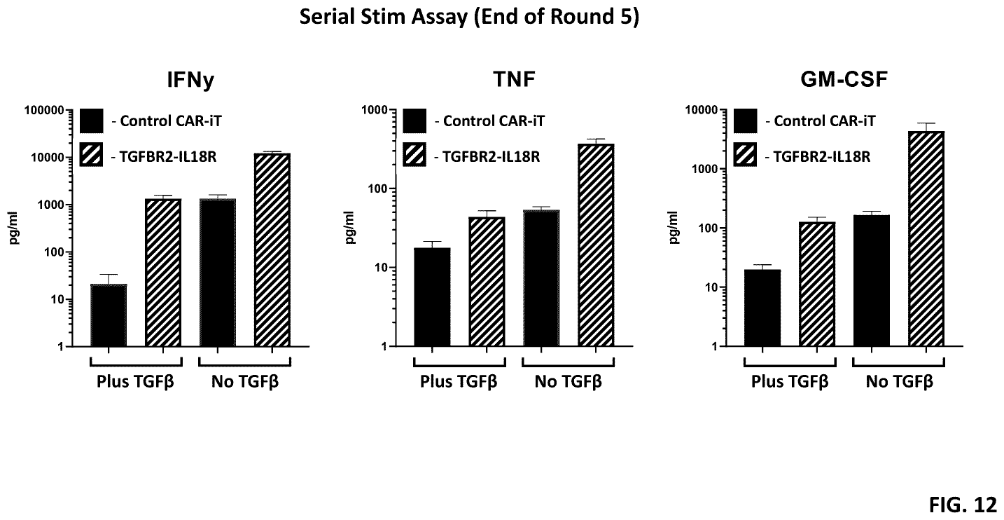

Furthermore, supernatants from round 5 of the serial stimulation assay were collected and tested via an MSD assay for cytokines, such as IFNγ, TNFα, and GM-CSF. As shown in FIG. 12, in the presence of TGFβ, control CAR-iTs showed a dramatic reduction of cytokines in the co-culture media. In contrast, much higher concentrations of the tested cytokines were found in the co-cultures with the TGFβ-SRR CAR-iTs. Even in the presence of TGFβ, the various cytokines measured in the co-cultures of the TGFβR2-IL18R CAR-iTs were similar in amount to that found in the co-cultures of the control CAR-iTs without TGFβ spiked-in. The enhanced cytokine production in the serial stimulation by the target cell in the presence of TGFβ demonstrates the ability of TGFβR2-IL18R CAR-iTs to maintain effector function in the presence of TGFβ, a representative immunosuppressive feature of a solid tumor environment.

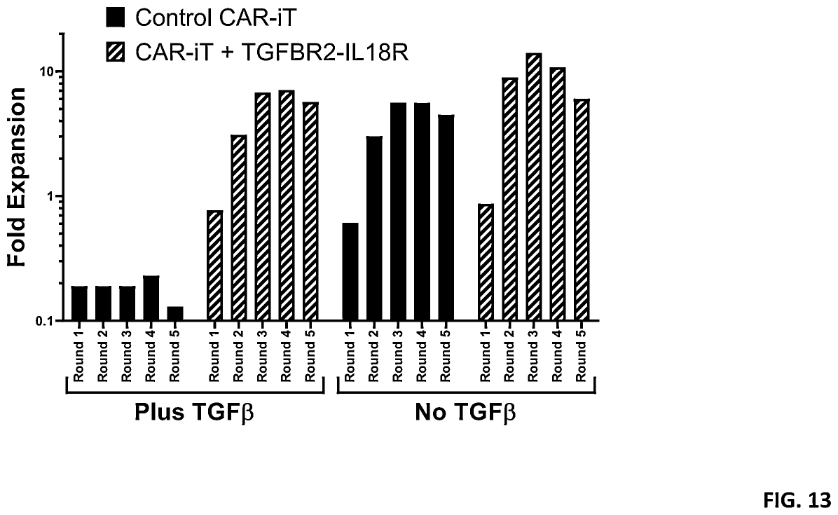

To test if CAR-iT cells with the TGFβ-SRR are capable of expanding after target cell stimulation in a solid tumor environment, control or TGFβR2-IL18R CAR-iTs were subjected to 5 rounds of co-culture with target cells in the presence or absence of TGFβ. Cells were harvested at the end of each round during this serial stimulation assay, and the number of effector cells was determined using counting beads and flow cytometry. As shown in FIG. 13, control CAR-iT cells (black bars) showed a lack of expansion in the presence of TGFβ (Plus TGFβ), while the TGFβR2-IL18R CAR-iTs (hashed bars) exhibited a normal expansion profile similar to the control CAR-iTs serially stimulated in the absence of TGFβ (No TGFβ). The TGFβR2-IL18R CAR-iTs also exhibited robust expansion in the absence of TGFβ with a higher fold expansion observed in rounds 2, 3, and 4 compared to the control CAR-iTs. Therefore, the TGFβ-SRR provides CAR-iT cells with improved expansion even with multiple rounds of target cell stimulation and in the presence of TGFβ, typical of a solid tumor environment.

A further designed and tested TGFβ-SRR is one comprising a fragment of the cytoplasmic domain of IL12Rβ2 in addition to the ectodomain of TGFβR2. The fragment of the cytoplasmic domain of IL12Rβ2 is represented by SEQ ID NO: 4. The TGFβR2-trIL12Rβ2 comprises an exemplary amino acid sequence represented by SEQ ID NO: 7, with understanding by one skilled in the art that the transmembrane domain sequence of which could vary or be replaced with a transmembrane domain of another transmembrane protein.

SEQ ID NO: 4
SDPKPENPACPWTVLPAGDLPTHDGYLPSNIDDLPSHEAPLADSLEELEPQ
SEQ ID NO: 7
TIPPHVQKSVNNDMIVTDNNGAVKFPQLCKFCDVRESTCDNQKSCMSNCSITSICEKPQEVCVAVWRKND
ENITLETVCHDPKLPYHDFILEDAASPKCIMKEKKKPGETFFMCSCSSDECNDNIIFSEEYNTSNPDLLL
VIFQVTGISLLPPLGVAISVIIIFYCYRVNSDPKPENPACPWTVLPAGDLPTHDGYLPSNIDDLPSHEAP
LADSLEELEPQ

(TGFβR2 ectodomain—transmembrane domain sequence—IL12Rβ2 endodomain fragment)

### Example 4—iPSC-Derived NK Cells Engineered with TGFβ-SRR Exhibit Enhanced Performance Against Solid Tumors

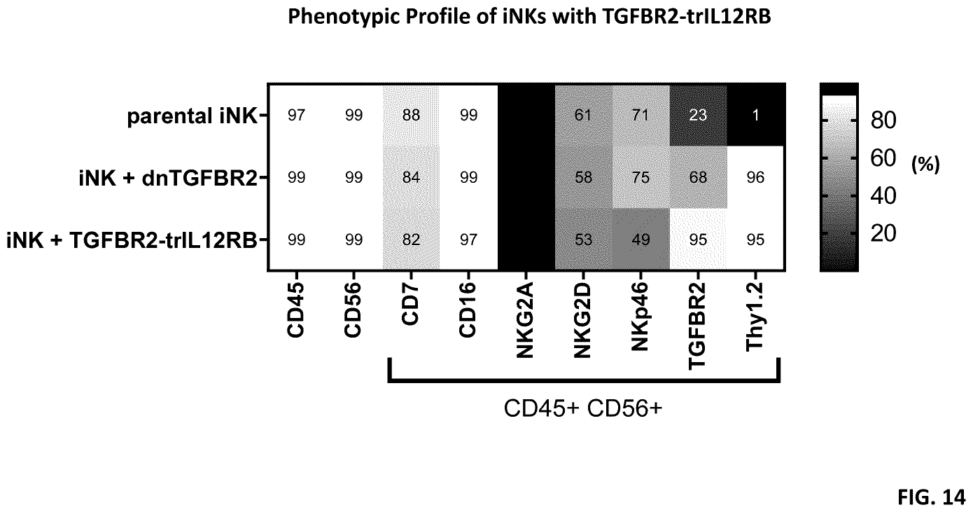

A bicistronic donor cassette comprising the polynucleotides encoding the TGFβR2-trIL12Rβ redirector receptor and hnCD16 was inserted in the CD38 locus of iPSCs using CRISPR/Cas9. The bulk-engineered iPSCs were sorted and differentiated into iNK cells in this experiment using the methods described in this application. Flow cytometry was used to detect surface expression of the indicated NK markers on iNK cells that were differentiated from iPSCs (FIG. 14). The iNK cells expressing dominant negative TGFβR2 (dnTGFβR2) or the TGFβR2-trIL12Rβ redirector receptor exhibit a phenotypic profile similar to that of the parental iNK. This indicates compatibility of the TGFβR2-trIL12Rβ transgene with the iNK differentiation process. Only the markers associated with the donor cassette, TGFβR2 and Thy1.2, are different in the TGFβR2-trIL12Rβ expressing iNKs, as expected.

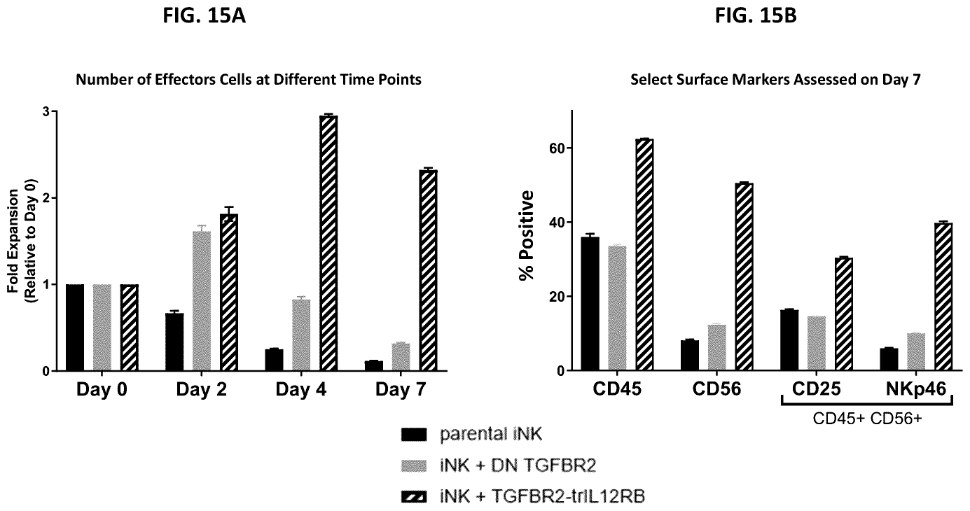

The parental iNKs, the dnTGFβR2 expressing iNKs, and the TGFβR2-trIL12R$ expressing iNKs were then each co-cultured with K562 target cells at an effector to target ratio of 1:1. 20 ng/mL of TGFβ was added to the co-cultures on days 0, 2, and 4. Flow cytometry was used to assess effector cell numbers on days 2, 4, and 7 of the co-culture. As shown in FIG. 15A, both the parental iNKs and the dnTGFβR2 iNKs showed progressive loss in effector cell numbers over the 7 days of co-culture. However, the iNK cells expressing the TGFβR2-trIL12Rβ redirector receptor showed robust expansion and persistence even until day 7 of the assay (FIG. 15A). Without being limited by theory, this increased performance over the dnTGFβR2 iNKs could be due to the redirection of TGFβ signals toward the IL12 signaling pathway through the cytoplasmic domain fragment of the IL12Rβ. Cells from day 7 were further analyzed by flow cytometry for the NK phenotypic markers (CD45 and CD56) or activation markers (CD25 and NKp46). As shown in FIG. 15B, the parental iNKs and the dnTGFβR2 iNKs showed similar proportions of cells expressing the selected surface markers, whereas the TGFβR2-trIL12Rβ iNK cells showed significantly increased proportions of cells that retained CD45 and CD56 expression. It is also noted that the TGFβR2-trIL12R$ iNKs showed a more activated profile based on the percentage of effectors expressing CD25 and NKp46 (FIG. 15B).

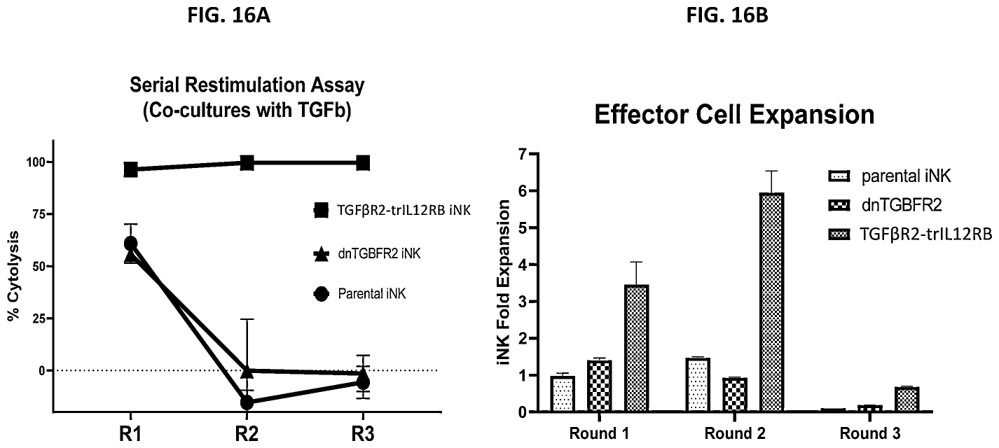

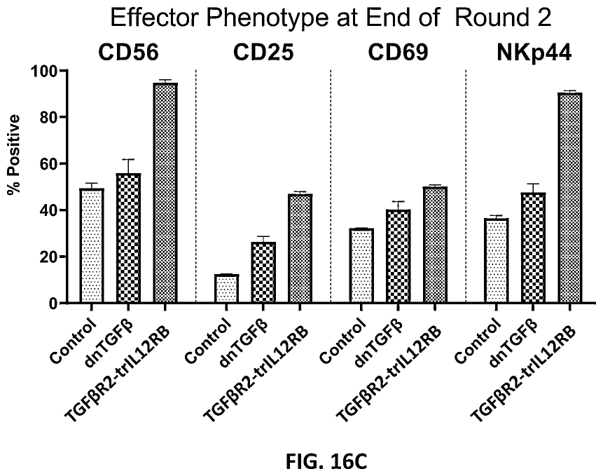

Further, the parental iNKs, the dnTGFβR2 expressing iNKs, and the TGFβR2-trIL12Rβ expressing iNKs were each co-cultured with Raji tumor target cells at an effector to target ratio of 1:1. 20 ng/mL of TGFβ was added to the co-cultures on days 0, 2, and 4, and innate killing capacity toward the target cells was measured by flow cytometry over three rounds. As shown in FIG. 16A, both the parental iNKs and the dnTGFβR2 iNKs showed a significant loss in innate killing capacity afler the first round of stimulation, while the TGFβR2-trIL12Rβ iNK cells maintained the ability to kill targets over all three rounds of co-culture. At the end of each round of co-culture, flow cytometry was also used to determine the expansion of the effector cells. As shown in FIG. 16B, iNK cells expressing the TGFβR2-trIL12Rβ redirector receptor showed robust expansion compared to the parental iNKs and the dnTGFβR2 iNKs, suggesting that the TGFβR2-trIL12Rβ redirector receptor improves effector cell expansion in the presence of target cells.

Cells from the end of round 2 of restimulation were further analyzed by flow cytometry for the NK phenotypic marker (CD56) and activation markers (CD25, CD69 and NKp44). As shown in FIG. 16C, the TGFβR2-trIL12Rβ iNK cells showed significantly increased proportions of cells expressing the selected surface activation markers compared to the parental iNKs (control) and the dnTGFβR2 iNKs. As in the previous experiment, the TGFβR2-trIL12Rβ iNKs showed a more activated profile based on the percentage of effectors expressing activation markers (FIG. 16C).

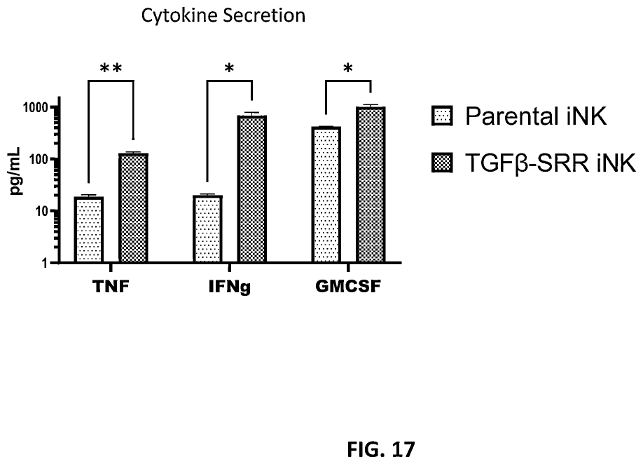

The TGFβR2-trIL12Rβ iNK cells and Parental iNK were co-cultured overnight with MDA-MB-231 cancer cells at a ratio of 5:1 in the presence of 20 ng/ml TGFβ and a monoclonal PDL1 antibody (mAb). The MDA-MB-231 breast cancer cell line overexpresses PDL1. Avelumab is an ADCC-competent monoclonal antibody (mAb) capable of binding to the exogenous CD16 comprised in the parental iNK cells and the engineered variants thereof for the iNK cells to carry our ADCC mediated cytotoxicity against the target cancer cell. After overnight co-culture, supernatants were harvested, centrifuged, and loaded onto a multianalyte cartridge to quantify secretion of the cytokines TNF, IFNγ, and GMCSF using the Ella platform (Bio-Techne, Minneapolis, MN) (FIG. 17).

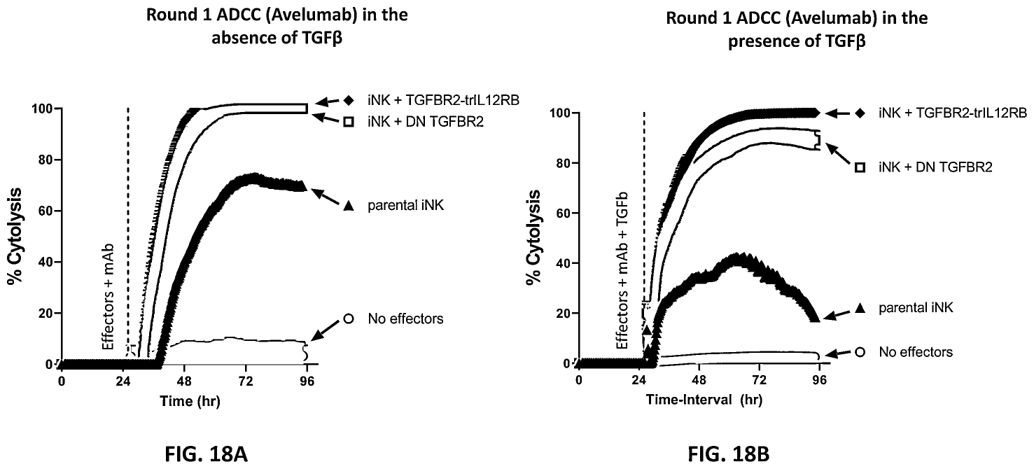

An xCelligence-based ADCC assay was used to determine the ADCC capacity of the different iNK effectors. As shown in FIG. 18A, in the absence of effectors, addition of the mAb alone (10 μg/mL Avelumab) was not sufficient to induce cytolysis of target cells by any line of iNK cells. Addition of effectors (E:T ratio of 5:1 based on target cell seeding numbers) to the culture resulted in cytolysis of the target cells by each tested iNK cell line. Parental iNK effectors exhibited ADCC toward the MDA-MB-231 target cells, but the iNKs expressing either dnTGFβR2 or the TGFβR2-trIL12R43 showed much better cytolytic activity.

Without being limited by theory, it is possible that blocking TGFβ signaling during the iNK differentiation process can result in iNKs that have improved effector function, such that both dnTGFβR2 and TGFβR2-trIL12Rβ pi iNKs show better ADCC even in the absence of TGFβ. However, when 20 ng/mL TGFβ was added to the culture medium, the xCelligence-based ADCC assay showed that with TGFβ spike-in, the parental iNKs had a dramatic loss in ADCC capacity, the dnTGFβR2 iNKs decreased in overall ADCC activity, but the TGFβR2-trIL12Rβ iNKs still exhibited complete cytolysis of target cells by the end of the assay (FIG. 18B).

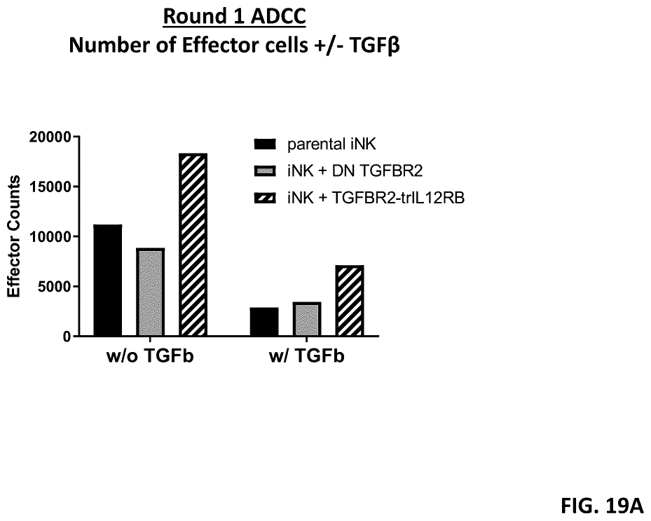

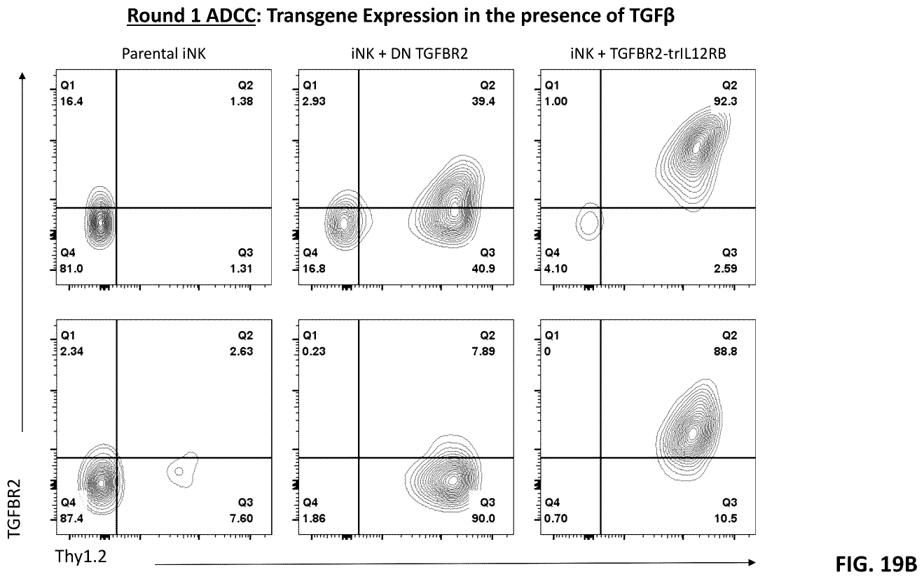

In addition, cells were harvested at the end of the first round of co-culture with the MDA-MB-231 target cells and analyzed by flow cytometry to determine the number of effector cells remaining and transgene expression at the given time point. Without being limited by theory, the enhanced persistence of TGFβR2-trIL12Rβ expressing iNKs compared to iNKs expressing dnTGFβR2 could be attributed to the TGFβR2-trIL12Rβ redirector receptor not only blocking TGFβ signaling but also initiating the IL12 signaling cascade (FIG. 19A). Additionally, as shown in FIG. 19B, the TGFβR2-trIL12Rβ construct seems to be more stable and highly expressed on the surface of iNK cells in the presence of TGFβ, a hallmark of the suppressive solid tumor environment.

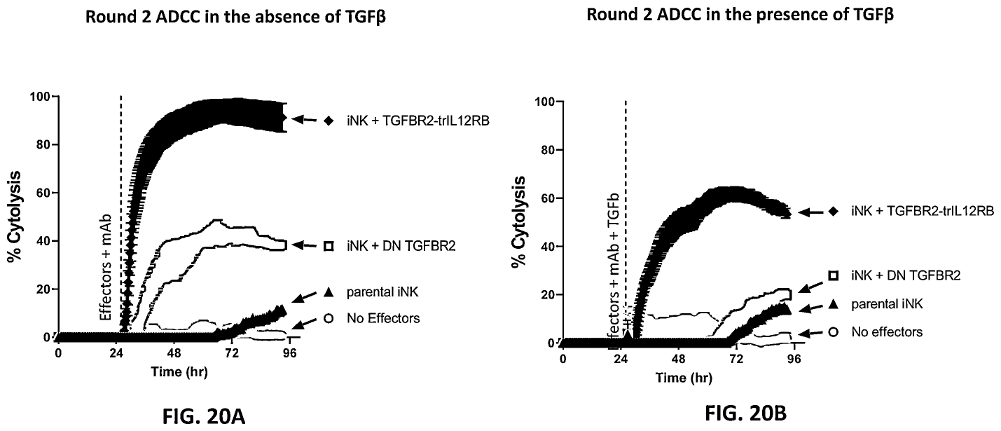

With the same set-up as FIG. 18A, at the end of the Round 1 co-culture, the effector cells were transferred to a second xCelligence plate with MDA-MB-231 targets. Following addition of effectors and Avelumab, % cytolysis of Round 2 was measured accordingly. FIG. 20A shows that even in the absence of TGFβ the parental iNKs lost effector function, as almost no ADCC activity was observed toward the target cells. The dnTGFβR2 iNKs retained some amount of ADCC activity, albeit greatly reduced when compared to the TGFβR2-trIL12R3 iNKs, which were able to lyse the target cells almost completely. Without being limited by theory, a certain level of tonic signaling from the TGFβR2-trIL12Rβ construct could result in iNKs expressing this transgene to have robust effector function even after multiple rounds of target stimulation. In the presence of TGFβ, however, even the dnTGFβR2 iNKs lost almost all ability to lyse target cells just like the parental iNKs (FIG. 20B). And while the TGFβR2-trIL12Rβ iNKs also showed reduced ADCC capacity in the presence of TGFβ, there was a clear benefit in having the TGFβR2-trIL12Rβ transgene over the dnTGFβR2 transgene in overcoming the suppression effect of TGFβ. Such observations in NK cells where expression of TGFβR2-trIL12Rβ result in enhanced effector cell expansion, persistence, and effector function could be expected in T cells, as the IL12 signaling pathway is considered as important in T cells as it is in NK cells.

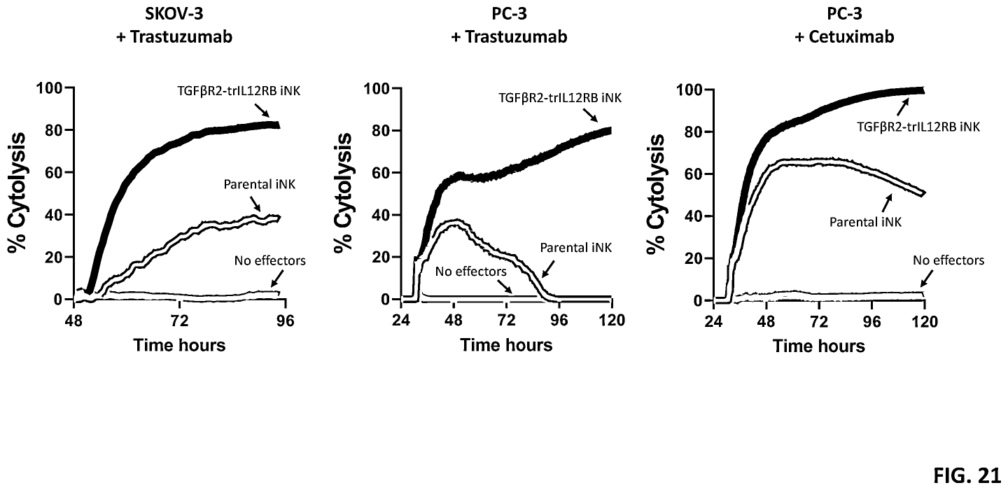

To further assess ADCC capacity of the different iNK effectors, the assay was expanded to additional solid cancer cell models including the SKOV3 ovarian cell tumor line and the PC-3 prostate cancer cell line. Since SKOV3 cancer cells overexpress HER2, trastuzumab (an anti-HER2 antibody commercially known as Herceptin™) was selected as an exemplary ADCC-competent mAb against SKOV3 targets, while trastuzumab and cetuximab (an anti-EGFR antibody) were selected as the exemplary mAbs for the PC-3 targets in view of their expression of both HER2 and EGFR. As shown in FIG. 21, the iNKs expressing the TGFβR2-triL12Rβ showed much better cytolytic activity toward all targets, compared to Parental iNK effectors.

In summary, with regard to adapting the TGFβ-SSR to NK effector cells, it was shown that the cytokine signaling pathways incorporated into the TGFβ-SSR can enable activation of cytokine signaling in the presence of TGFβ. When the cytokine signaling pathways were activated by TGFβ, as shown, the anti-tumor activity of the effector cell was enhanced while attenuating the suppressive effects of TGFβ. NK cells expressing the TGFβ-SRR exhibited enhanced innate killing capacity, proliferation, and activation profile in the presence of tumor targets and TGFβ. NK cells expressing a TGFβ-SRR also exhibited enhanced ADCC and cytokine production in co-cultures with solid tumor lines in the presence of TGFβ. Also shown here is that iPSCs can be engineered with a TGFβ-SRR and subsequently differentiated into mature and phenotypically typical NK cells. In comparison, although a dominant negative TGFβR2 reduces TGFβ signaling that is suppressive to effector cell anti-tumor activity, it however, does not provide additional advantages including, but not limited to, effector cell enhanced innate killing capacity, proliferation and activation, and enhanced ADCC and cytokine production, which synergistically lead to the superior effector function of the cells expressing a TGFβ-SRR as disclosed herein.

The data presented herein demonstrate that select cytokine signaling pathways may be used to enhance the anti-tumor function of both iT and iNK cells. Furthermore, turning on these signals may allow iT and iNK cells to resist TGFβ-mediated suppression of effector function. The strategy of fusing select cytokine endodomains to a TGFβR2 ectodomain can make use of TGFβ in the environment to turn on a beneficial signaling pathway that can then allow the effector cells to function at a higher level than they could otherwise.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

1. A cell or a population thereof, wherein:
(i) the cell is (a) an immune cell; (b) an induced pluripotent cell (iPSC), a clonal iPSC, or an iPS cell line cell; or (c) a derivative cell obtained from differentiating the iPSC;
(ii) the cell comprises an exogenous polynucleotide encoding a signaling redirector receptor (SRR) that comprises a partial or full peptide of an extracellular domain (ECD) of a signaling receptor and a partial or full peptide of an intracellular domain (ICD) of a cytokine receptor, wherein the signaling receptor and the cytokine receptor are different molecules;
(iii) the cell has improved resistance to cytokine immunosuppression in an adoptive cell therapy for solid tumors; and
(iv) the cell optionally further comprises one or more of:
an exogenous polynucleotide encoding a CAR (chimeric antigen receptor)
an exogenous polynucleotide encoding a CD16 or a variant thereof,
CD38 knockout; and
an exogenous polynucleotide encoding a cytokine signaling complex comprising a partial or full peptide of a cell surface expressed exogenous cytokine and/or a receptor thereof.

2. The cell or population thereof of claim 1, wherein the signaling redirector receptor comprises:
(a) a partial or full peptide of the extracellular domain (ECD) of a signaling receptor comprising transforming growth factor beta receptor (TGFβR), programmed cell death 1 (PD1), CTLA4, IL10R, IL4R, or any combination thereof, and
(b) a partial or full peptide of the intracellular domain (ICD) of a cytokine receptor comprising IL2Rβ, IL12Rβ, IL18Rβ, IL21R, or any combination thereof.

3. The cell or population thereof of claim 2, wherein the signaling receptor comprises TGFβR2, wherein the signaling redirector receptor is a TGFβ-SRR, and
(a) wherein the cytokine receptor is IL2Rβ, thereby forming a TGFβR2-IL2Rβ signaling redirector receptor; or
(b) wherein the cytokine receptor is IL12Rβ, thereby forming a TGFβR2-IL12Rβ signaling redirector receptor; or
(c) wherein the cytokine receptor is IL18Rβ, thereby forming a TGFβR2-IL18Rβ signaling redirector receptor; or
(d) wherein the cytokine receptor is IL21R, thereby forming a TGFβR2-IL21R signaling redirector receptor.

4. The cell or population thereof of claim 3, wherein:
(a) the intracellular domain (ICD) of IL2Rβ comprises an amino acid sequence represented by SEQ ID NO: 2; or
(b) the intracellular domain (ICD) of IL12Rβ comprises an amino acid sequence represented by SEQ ID NO: 3 or SEQ ID NO: 4; or
(c) the intracellular domain (ICD) of IL18Rβ comprises an amino acid sequence represented by SEQ ID NO: 5; or
(d) the intracellular domain (ICD) of IL21Rβ comprises an amino acid sequence represented by SEQ ID NO: 6; or
(e) the extracellular domain (ECD) of TGFβR comprises an amino acid sequence represented by SEQ ID NO: 1.

5. The cell or population thereof of claim 3, wherein the TGFβR2-IL12Rβ signaling redirector receptor comprises an amino acid sequence having a sequence identity of at least 80%, 85%, 90%, 95%, or 97%, 98%, or 99% to a sequence represented by SEQ ID NO: 7, wherein an amino acid sequence represented by SEQ ID NO: 8 comprised in SEQ ID NO: 7 is variable.

6. The cell or population thereof of claim 1, wherein the cell further comprises:
(i) HLA-I deficiency and/or HLA-II deficiency;
(ii) introduction of HLA-G or non-cleavable HLA-G;
(iii) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(iv) introduction of at least one of HLA-E, 4-1BBL, CD3, CD4, CD8, CD16, CD47, CD64, CD113, CD131, CD137, CD80, PDL1, A_{2A}R, TCR, chimeric fusion receptor (CFR), Fc receptor, an antibody or functional variant or fragment thereof, a checkpoint inhibitor, an engager, and surface triggering receptor for coupling with bi- or multi-specific or universal engagers.

7. The cell or population thereof of claim 1, wherein the cell comprises HLA-I deficiency and/or HLA-II deficiency; and optionally,
wherein the cell comprises an exogenous polynucleotide encoding HLA-G, HLA-E, or a variant thereof.

8. The cell or population thereof of claim 7, wherein the HLA-I deficiency comprises deletion or disruption of at least one of: B2M, TAP1, TAP2, and Tapasin; or
wherein the HLA-II deficiency comprises deletion or disruption of at least one of: CIITA, RFX5, RFXAP, and RFXANK.

9. The cell or population thereof of claim 1, wherein the derivative cell:
(a) comprises a derivative CD34⁺ cell, a derivative hematopoietic stem and progenitor cell, a derivative hematopoietic multipotent progenitor cell, a derivative T cell progenitor, a derivative NK cell progenitor, a derivative T cell, a derivative NKT cell, a derivative NK cell, a derivative B cell, or a derivative effector cell having one or more functional features that are not present in a counterpart primary T, NK, NKT, and/or B cell;
(b) is an allogeneic effector cell, wherein the effector cell is a derivative NK cell or a derivative T cell having at least one of the following characteristics comprising:
(i) improved persistency and/or survival;
(ii) increased resistance to activated recipient immune cells;
(iii) increased cytotoxicity;
(iv) improved tumor penetration;
(v) enhanced or acquired ADCC;
(vi) enhanced ability in migrating, and/or activating or recruiting bystander immune cells, to tumor sites;
(vii) enhanced ability to reduce tumor immunosuppression;
(viii) improved ability in rescuing tumor antigen escape; and
(ix) reduced fratricide,
in comparison to its native counterpart cell obtained from peripheral blood, umbilical cord blood, or other donor tissues.

10. The cell or population thereof of claim 1, wherein the exogenous CD16 comprises at least one of:
(a) a high affinity non-cleavable CD16 (hnCD16) or a variant thereof;
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

11. The cell or population thereof of claim 10, wherein:
(a) the non-native transmembrane domain is derived from a CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide;
(b) the non-native stimulatory domain is derived from a CD27, CD28, 4-1BB, OX40, ICOS, PD-1, LAG-3, 2B4, BTLA, DAP10, DAP12, CTLA-4, or NKG2D polypeptide;
(c) the non-native signaling domain is derived from a CD30, 2B4, DAP10, DAP12, DNAM1, CD137 (4-1BB), IL21, IL7, IL12, IL15, NKp30, NKp44, NKp46, NKG2C, or NKG2D polypeptide; or
(d) the non-native transmembrane domain is derived from NKG2D, the non-native stimulatory domain is derived from 2B4, and the non-native signaling domain is derived from CD3ζ.

12. The cell or population thereof of claim 1, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with a cytokine signaling complex comprising a partial or full peptide of a cell surface expressed exogenous cytokine and/or a receptor thereof, optionally in separate constructs or in a bi-cistronic construct;
(ix) co-expressed with a checkpoint inhibitor, optionally in separate constructs or in a bi-cistronic construct; and/or
(x) optionally inserted at:
(1) a TRAC or a TRBC locus, and/or is driven by an endogenous promoter of TCR, and/or the TCR is knocked out by the CAR insertion;
(2) a safe harbor locus; or
(3) a gene locus intended for disruption.

13. The cell or population thereof of claim 1, wherein the CAR is:
(i) specific to at least one CD19, BCMA, B7H3, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1; and/or
(ii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), Mucin 1 (Muc-1), Mucin 16 (Muc-16), Mesothelin (MSLN), NKCSI, NKG2D ligands, c-Met, cancer-testis antigen NYESO-1, oncofetal antigen (h5T4), PRAME, prostate stem cell antigen (PSCA), PRAME prostate-specific membrane antigen (PSMA), tumor associated glycoprotein 72 (TAG-72), TIM-3, TRBC1, TRBC2, vascular endothelial growth factor R2 (VEGFR2), Wilms tumor protein (WT-1), and a pathogen antigen.

14. The cell or population thereof of claim 1, wherein the cytokine signaling complex comprises:
(a) a partial or full peptide of at least one of IL2, 114, IL6, IL7, IL9, IL10, I111, IL12, IL15, IL18, IL21, and/or respective receptor(s) thereof; or
(b) at least one of:
(i) co-expression of IL15 and IL15Rα with a self-cleaving peptide in-between;
(ii) a fusion protein of IL15 and IL5Rα;
(iii) an IL15/IL15Rα fusion protein with intracellular domain of IL15Rα truncated (IL15Δ);
(iv) a fusion protein of IL15 and membrane bound Sushi domain of IL15Rα;
(v) a fusion protein of IL15 and IL15Rβ;
(vi) a fusion protein of IL15 and common receptor γC, wherein the common receptor γC is native or modified; and
(vii) a homodimer of IL15Rβ;
wherein any one of (b)(i)-(vii) is optionally co-expressed with a CAR in separate constructs or in a bi-cistronic construct; or
(c) at least one of:
(i) a fusion protein of IL7 and IL7Rα;
(ii) a fusion protein of IL7 and common receptor γC, wherein the common receptor γC is native or modified; and
(iii) a homodimer of IL7Rβ,
wherein any one of (cxi)-(iii) is optionally co-expressed with a CAR in separate constructs or in a bi-cistronic construct;
and optionally,
(d) is transiently expressed.

15. The cell or population thereof of claim 1, wherein the cell is a derivative NK or a derivative T cell, wherein the derivative NK cell is capable of recruiting and/or migrating T cells to tumor sites, and wherein the derivative NK cell or the derivative T cell is capable of reducing tumor immunosuppression in the presence of one or more checkpoint inhibitors.

16.-17. (canceled)

18. The cell or population thereof of claim 1, wherein the cell comprises:
(i) one or more exogenous polynucleotides integrated in one safe harbor locus or locus intended for disruption; or
(ii) more than two exogenous polynucleotides integrated in different safe harbor loci or loci intended for disruption.

19. The cell or population thereof of claim 18, wherein the safe harbor locus or loci comprises at least one of AAVS1, CCR5, ROSA26, collagen, HTRP, H11, GAPDH, TCR or RUNX1; or wherein the gene locus or loci intended for disruption comprises at least one of B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD58, CD54, CD56, CIS, CBL-B, SOCS2, PD1, CTLA4, LAG3, TIM3, or TIGIT.

20. A composition comprising the cell or population thereof of claim 1.

21. The composition of claim 20, further comprising one or more therapeutic agents.

22. The composition of claim 21, wherein the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

23. The composition of claim 22, wherein:
(i) the checkpoint inhibitor comprises:
(a) one or more antagonists to checkpoint molecules comprising PD-1, PDL-1, TIM-3, TIGIT, LAG-3, CTLA-4, 2B4, 4-1BB, 4-1BBL, A2AR, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR;
(b) one or more of atezolizumab, avelumab, durvalumab, ipilimumab, IPH4102, IPH43, IPH33, lirimumab, monalizumab, nivolumab, pembrolizumab, and their derivatives or functional equivalents;
(c) at least one of atezolizumab, nivolumab, and pembrolizumab; or
(ii) the therapeutic agents comprise one or more of venetoclax, azacitidine, and pomalidomide.

24. The composition of claim 22, wherein the antibody comprises:
(a) an anti-CD20 antibody, an anti-HER2 antibody, an anti-CD52 antibody, an anti-EGFR antibody, an anti-CD123 antibody, an anti-GD2 antibody, or an anti-PDL1 antibody; or
(b) one or more of rituximab, veltuzumab, ofatumumab, ublituximab, ocaratuzumab, obinutuzumab, trastuzumab, pertuzumab, alemtuzumab, cetuximab, dinutuximab, avelumab, daclizumab, basiliximab, M-A251, 2A3, BC69, 24204, 22722, 24212, MAB23591, FN50, 298614, AF2359, CYlG4, DF1513, bivatuzumab, RG7356, G44-26, 7G3, CSL362, elotuzumab, and their humanized or Fc modified variants or fragments and their functional equivalents and biosimilars thereof.

25. The composition of claim 20, wherein the composition comprises an engager, and further wherein:
(i) the engager comprises a bispecific T cell engager (BiTE);
(ii) the engager comprises a bispecific killer cell engager (BiKE); or
(iii) the engager comprises a tri-specific killer cell engager (TriKE); or
(iv) the engager comprises:
(a) a first binding domain recognizing an extracellular portion of CD3, CD5, CD16, CD28, CD32, CD33, CD64, CD89, NKG2C, NKG2D, or any functional variants thereof of the cell or a by-stander immune effector cell; and
(b) a second binding domain specific to an antigen comprising any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

26. Therapeutic use of the composition of claim 20 by introducing the composition to a subject in need of an adoptive cell therapy, wherein the subject has an autoimmune disorder, a hematological malignancy, a solid tumor, cancer, or a virus infection.

27. A master cell bank (MCB) comprising the clonal iPSC of claim 1.

28. A method of manufacturing the derivative cell of claim 1, wherein the derivative cell is an effector cell, and the method comprises:
differentiating a genetically engineered iPSC, wherein the iPSC comprises the exogenous polynucleotide encoding the signaling redirector receptor;
thereby providing the effector cell with improved resistance to cytokine immunosuppression in an adoptive cell therapy for solid tumors.

29. The method of claim 28, wherein the signaling redirector receptor comprises:
(a) a partial or full peptide of the extracellular domain (ECD) of a signaling receptor comprising transforming growth factor beta receptor (TGFβR), programmed cell death 1 (PD1), CTLA4, IL10R, IL4R, or any combination thereof; and
(b) a partial or full peptide of the intracellular domain (ICD) of a cytokine receptor comprising IL2Rβ, IL12Rβ, IL18Rβ, IL21Rβ, or any combination thereof;
wherein the genetically engineered iPSC is a single cell, a clonal cell, or a cell line cell.

30. The method of claim 29, wherein the signaling receptor comprises TGFβR, and
(a) wherein the cytokine receptor is IL2Rβ, thereby forming a TGFβR2-IL2Rβ signaling redirector receptor; or
(b) wherein the cytokine receptor is IL12Rβ, thereby forming a TGFβR2-IL12Rβ signaling redirector receptor; or
(c) wherein the cytokine receptor is IL18Rβ, thereby forming a TGFβR2-IL18Rβ signaling redirector receptor; or
(d) wherein the cytokine receptor is IL21R, thereby forming a TGFβR2-IL21R signaling redirector receptor.

31.-32. (canceled)

33. The method of claim 28, further comprising:
genomically engineering an iPSC to knock in: (a) the exogenous polynucleotide encoding the signaling redirector receptor; and optionally, (b) the exogenous polynucleotide encoding the chimeric antigen receptor (CAR); and optionally (c) the exogenous polynucleotide encoding the CD16 or a variant thereof; and optionally further comprising genomically engineering the iPSC:
(i) to knock out CD38,
(ii) to knock out one or both of B2M and CIITA,
(iii) to introduce HLA-G or non-cleavable HLA-G, and/or
(iv) to introduce a cytokine signaling complex comprising the partial or full peptide of the cell surface expressed exogenous cytokine and/or receptor thereof.

34. The method of claim 33, wherein the genomically engineering comprises targeted editing.

35.-51. (canceled)
